(19) United States
(12) Patent Application Publication
(10) Pub. No.: US 2014/0157457 A1
(43) Pub. Date: Jun. 5, 2014

(54) PLANT PROTEASE

(71) Applicants: Plant Bioscience Limited, Norwich (GB); Universidad Nacional De La Plata, La Plata (AR); Consejo Nacional De Investigaciones Cientificas Y Tecnicas, Buenos Aires (AR)

(72) Inventors: Juan Jose Guiamet, Buenos Aires (AR); Dana Ethel Martinez, Buenos Aires (AR)

(73) Assignees: Plant Bioscience Limited, Norwich (GB); Universidad Nacional De La Plata, La Plata (AR); Consejo Nacional De Investigaciones Cientificas Y Tecnicas, Buenos Aires (AR)

(21) Appl. No.: 13/974,143

(22) Filed: Aug. 23, 2013

Related U.S. Application Data
(63) Continuation-in-part of application No. PCT/GB2012/050420, filed on Feb. 24, 2012.

(30) Foreign Application Priority Data
Feb. 25, 2011 (GB) 1103270.3
Apr. 15, 2011 (GB) 1106428.4

(51) Int. Cl. C12N 15/82

(52) U.S. Cl. CPC C12N 15/8261
USPC 800/290; 435/412; 435/419; 435/320.1; 536/23.6; 800/298; 800/306; 800/320.1; 800/320.2; 800/320.3

(57) ABSTRACT

The application relates to methods for increasing plant yield and transgenic plants with increased yield using a plant protease.

### RELATED APPLICATIONS AND INCORPORATION BY REFERENCE

This application is a continuation-in-part application of international patent application Serial No. PCT/GB2012/050420 filed 24 Feb. 2012, which published as PCT Publication No. WO 2012/114117 on 30 Aug. 2012, which claims benefit of GB patent application Serial No. 1103270.3 filed 25 Feb. 2011 and GB patent application Serial No. 1106428.4 filed 15 Apr. 2011.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### FIELD OF THE INVENTION

The invention relates to methods for increasing plant yield. It also relates to modified plant cells and plants which may comprise an inactivated or down-regulated senescence-associated plant subtilisin protease gene or a gene inhibiting this protease, resulting in increased yield as compared to non-transformed wild type plants or plant cells and to methods for producing such plant cells or plants.

### BACKGROUND OF THE INVENTION

Growing human population and environmental issues such as climate change pose significant challenges to agriculture. To meet the increasing demand, it is essential to improve crop productivity and yield (Rothstein 2007). Conventional breeding techniques have several drawbacks, as they are typically time consuming and labour intensive, restricted to variation naturally found in the species bred and result in plants that often contain heterogeneous genetic components that may not always result in the desirable trait being passed on from parent plants. Using biotechnology tools, it has been possible to specifically and rapidly modify plants by genetic engineering to improve economic, agronomic or horticultural traits.

For most crops there is a high correlation between seed number and yield (e.g., Diepenbrock 2000). Breeders have (mostly unconsciously) modified plant reproductive structures to improve seed number. Increased seed number may be due to more reproductive branches per plant, or more branched inflorescences. Plant branching is sensitive to environmental cues, but is genetically controlled, with shared genetic pathways between monocots and dicots (e.g. Doust, 2007). Reproductive branching represents one of the most important target traits for genetic improvement of crops. Some cereals like wheat and rice have been selected for multiple tillers with grain heads (e.g., Sharma 1995). Other monocots, e.g., maize, have been selected for reduction in axillary branches and an increase in the size in the main inflorescence. In dicots like soybean and rape-seed, seed yield correlates with the number of branches, pods per plant and seeds per pod (Diepenbrock 2000).

Branching and seed production occur under a complex regulation at different levels and by different factors, i.e., axillary meristem initiation and activity, inter branches and/or inter shoot competition, sink-source ratios, resource allocation between different fruits, rate of organ growth, etc. (e.g. Nooden and Penney 2001). In monocarpic species, reproductive growth and monocarpic senescence are intertwined, but this relationship is poorly understood. Several studies in diverse transgenic Arabidopsis lines show that “bushy” phenotypes often relate to a delay in monocarpic senescence, but also to lower seed yield or even infertility or flower sterility (for example Bleecker & Patterson, 1997).

There are a number of genes encoding for subtilisin proteases (or “subtilases”) in plants. In Arabidopsis, less than 10 have been characterised. Most of the plant subtilisin proteases characterized so far have been shown to have specific roles as regulatory players in diverse pathways of developmental programs and environmental responses. The SDD1 subtilase mediates cell signalling during stomata development, and the lack of its function results in abnormal stomata distribution and density (Berger & Altmann, 2000). The lack of function of the ALE1 subtilase leads to abnormal embryo development and embryo mortality (Watanabe et al., 2004). Other subtilisin proteases are involved in stress responses. The AtSP1 subtilase processes the transcription factor AtbZIP17 that in turn activates several salt stress response genes (Liu et al., 2007). The tomato P69B subtilase is induced and accumulates in the intercellular fluid in response to pathogen attack (e.g. Tornero et al. 1996).

Citation or identification of any document in this application is not an admission that such document is available as prior art to the present invention.

### SUMMARY OF THE INVENTION

The SASP (\ddot{S}enescence Ässociated \ddot{S}ubtilisin \ddot{P}rotease) gene described herein shows enhanced expression in senescing leaves. Also, transgenic plants in which the SASP gene function is knocked out show a delay in senescence and increased yield. The present invention is thus aimed at mitigating the problems described above and at providing methods and plants to improve crop yield.

The invention relates to methods for increasing plant yield by inactivating, repressing or down-regulating the activity of a plant SASP polypeptide or plant gene encoding a SASP polypeptide, and to plants with increased yield. Specifically, in a first aspect, the invention relates to a method for making a transgenic plant with increased yield which may comprise inactivating, repressing or down-regulating the activity of a SASP polypeptide or gene in a plant. In a further aspect, the invention relates to a plant obtained by these methods. In another aspect, the invention relates to an isolated plant nucleic acid which may comprise a sequence as shown in SEQ Id No. 1, 3, 4 or 5 or a functional variant, homologue or orthologue thereof. Also within the scope of the invention is a transgenic plant cell, plant or a part thereof with increased yield wherein the activity of a SASP gene or polypeptide is inactivated, repressed or down-regulated.

Accordingly, it is an object of the invention to not encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. §112, first paragraph) or the EPO (Article 83 of the EPC), such that Applicants reserve the right and hereby disclose a disclaimer of any previously described product, process of making the product, or method of using the product.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings.

FIG. 1. Proteolytic activity at different leaf developmental stages.

FIG. 1A. The zymogram reveals two protease activity bands which intensities increase during leaf senescence (arrow). Y=young leaf, S₁ and S₂=early and late senescing leaves, respectively. Samples loaded in the gel represent the same leaf area. MW: Molecular mass markers.

FIG. 1B. Section of a 2D zymogram and the corresponding region of a 2D conventional gel (silver stained) showing the two bands of interest observed as spots of activity (2D zymogram) and protein (silver stained 2D gel). The image on the left is a zoom of the zymogram shown in FIG. 1A.

FIG. 2. Expression pattern of AtSASP in different organs and cell types.

Results from microarrays analysis using the Genevestigator database, Gene Atlas Programme.

FIG. 3. Molecular and biochemical analysis of AtSASP-KO (SALK_{—}147962.44.25x T-DNA insertion line).

FIG. 3A. T-DNA insertion site on AtSASP DNA sequence and location of primers. LBb1 and RP primers amplify a 600 bp fragment starting at the T-DNA. LP and RP primers amplify a 1000 by fragment that corresponds to the WT allele.

FIG. 3B. PCR amplification products with LP-RP (1) and LBb1-RP (2) primers on WT and AtSASP-KO (SALK_{—}147962.44.25x line). DNA

FIG. 3C. Proteolytic activity in WT and AtSASP-KO leaf extracts. AtSASP-KO (SALK_{—}147962.44.25x line) plants lack AtSASP activity (arrows).

FIG. 4. Leaf senescence in AtSASP-KO plants. Chlorophyll content and leaf survival rate were considered as parameters of senescence.

FIG. 4A. Time-course of leaf senescence during the reproductive stage of plant development. Measurements were done on the ninth leaf counting from the top (both genotypes produce the same number of leaves).

FIG. 4B. Dark induced leaf senescence. Leaves were detached from the rosette and placed on moist paper towels in a plastic box. Leaves were arranged in order according to their position in the rosette (right panel). The leaves were kept in darkness for 1 week, and photographed every 2 days.

FIG. 5. Reproductive development in AtSASP-KO plants.

FIG. 5A. Total number of inflorescence branches produced per plant by the end of the reproductive development. Shading in the columns indicate first, second and third order branches. Branches of higher order were not included in this analysis. Values represent an average of 8 plants per genotype, 33.4±4.7 in WT and 48.2±10.3 in AtSASP-KO (p<0.05).

FIG. 5B. Number of siliques produced per plant by the end of the reproductive development. Colours in the columns indicates siliques produced by first, second, third order branches and the apical terminal part of the main inflorescence. Others: siliques developed in 4th and 5th order branches on the main inflorescence or on axillary inflorescences. Values represent an average of 8 plants per genotype, 585.4±21.4 and 412.8±43.6 siliques per plant, for AtSASP and WT plants, respectively (p<0.05).

FIG. 6. Time course analysis of inflorescence development.

FIG. 6A. Number of branches in AtSASP-KO and WT plants from the date of flowering through to the end of reproductive development. Each column represents the total number of branches per plant. Branches from axillary inflorescences (AI) and the main inflorescence (MI) are distinguished by colours. Data represent the average of 8 plants per genotype. Asterisks indicate statistically significant differences (p<0.05).

FIG. 6B Number of cauline leaves (CL) and branches in the main inflorescence (MI B) of AtSASP-KO and WT plants. Data represent the average of 8 plants per genotype. Asterisks indicate statistically significant differences (p<0.05).

FIG. 7. Multiple sequence analysis of AtSASP with other Arabidopsis subtilases and AtSASP homologues in oil-seed rape, rice and wheat.

FIG. 7A. Cluster tree obtained when nucleic acid and amino acid sequences are aligned. Generated with ClustalW.

FIG. 7B. Amino acid sequences alignment of AtSASP and SASPs in oil-seed rape, wheat and rice. Bold letters show conserved regions in SASPs, but which are not shared by the other subtilases included in this analysis.

FIG. 7C. Nucleic acid sequences alignment. Bold letters show nucleic acid sequences conserved in AtSASP, SASPs in oil-seed rape, wheat and rice, but which not shared by the other subtilases included in this analysis. Underlined letters indicate the sequences used for primer design.

FIG. 7D. PCR amplification of AtSASP and SASPs from oil-seed rape and rice.

PCR amplification of products from genomic DNA of Arabidopsis, oil-seed rape, and rice, obtained with primers designed based on conserved regions of nucleic acid sequences alignment (FIG. 7C, underlined letters).

FIG. 8. Expression of Os02g0779200, a rice SASP, is up-regulated in mature leaves respect to young leaves. The pictogram obtained from efpBrower software shows Os02g0779200 expression levels in different organs. The program uses a grey scale to represent gene expression, from the lowest (grey) to the highest (black) expression values. Os02g0779200 expression is high in mature leaves and in mature reproductive structures (panicles).

FIG. 9.A. Nucleic acid sequences alignment of AtSASP with the two putative SASPs in B. rapa, Bra027376 and Bra021529, generated with ClustallW. Underlined bold letters indicate the sequences used for primer design.

FIG. 9.B. PCR amplification of Bra027376 and Bra021529 from genomic DNA of B. rapa. The primers used for this amplification were designed based on specific regions of the genes (FIG. 9A).

FIG. 9.C. Real Time qPCR analysis of the two putative SASPs in B. rapa, Bra027376 and Bra021529. RNA samples, 1, 2, 3, 4, 5, were taken from young (Y), early senescing (S₁) and late senescing leaf tissue (S₂), at 45, 65 and 85 days after emergency (DAE) (Table 1). UBQ10 was used as reference.

### DETAILED DESCRIPTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

The practice of the present invention will employ, unless otherwise indicated, conventional techniques of botany, microbiology, tissue culture, molecular biology, chemistry, biochemistry and recombinant DNA technology, which are within the skill of the art. Such techniques are explained fully in the literature (for example Sambrook J et al, 1987).

The inventors have surprisingly found that mutant knock out plants which do not express the SASP gene produce higher yield. The inventors have identified a SASP in Arabidopsis (AtSASP) in a proteomic study designed to identify proteases with increased activity during leaf senescence. Genes that are homologous or orthologous to AtSASP are also described herein. Furthermore, conserved domains shared by SASPs from different plant species are described herein.

Therefore, the invention is not limited to AtSASP. The term “SASP” as used herein generally defines a plant subtilisin protease with increased activity during leaf senescence wherein plants with a loss of function or reduced function of the SASP gene or polypeptide show increased yield.

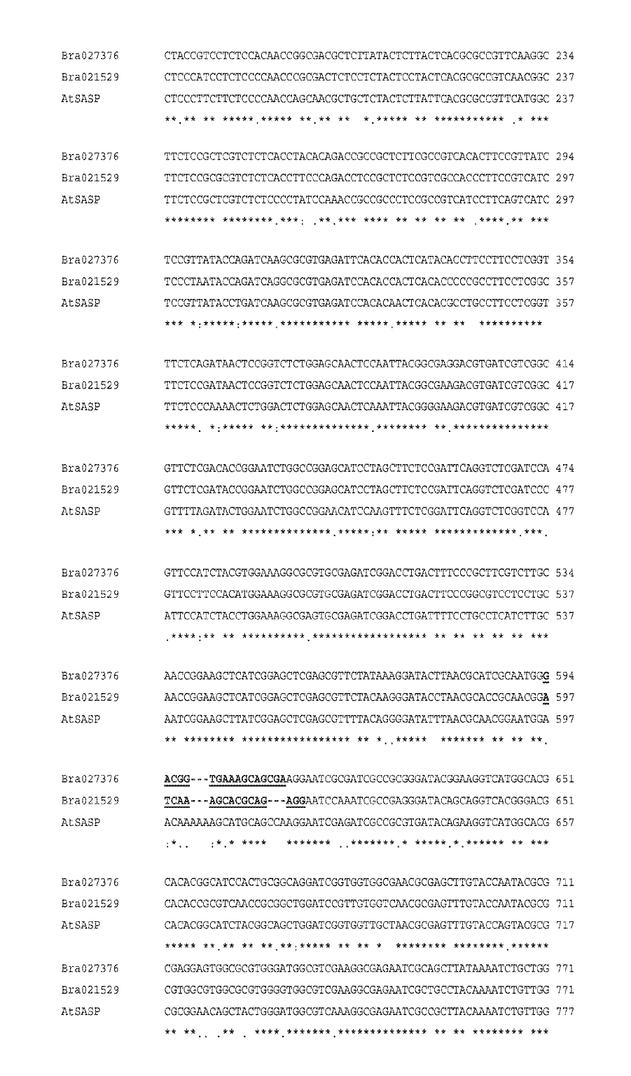

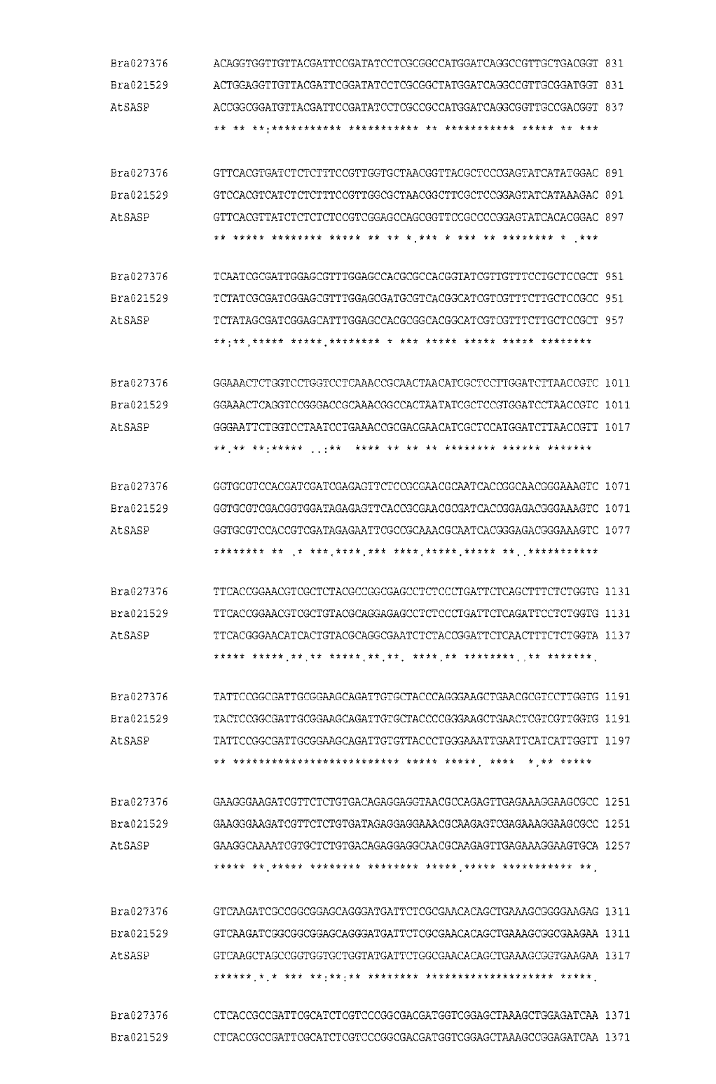

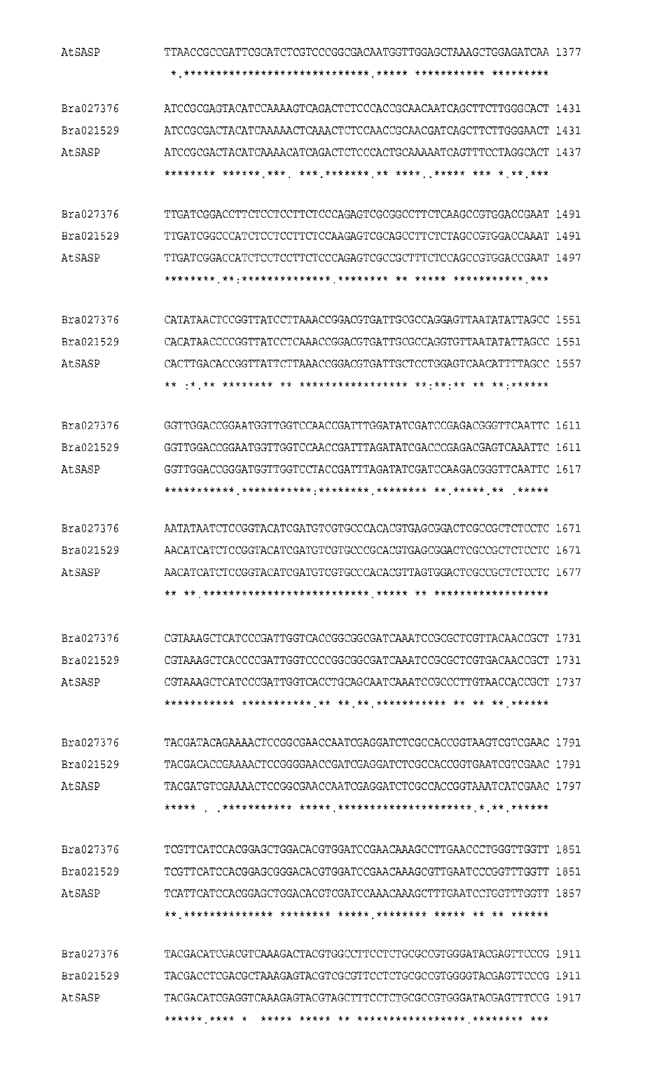

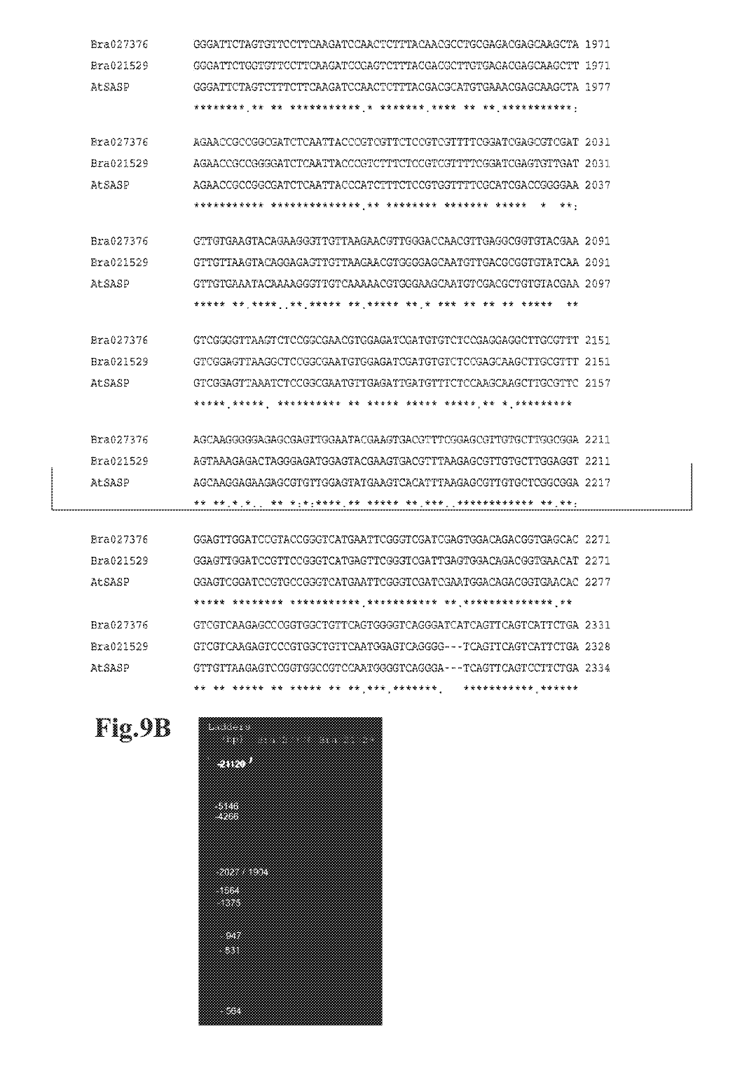

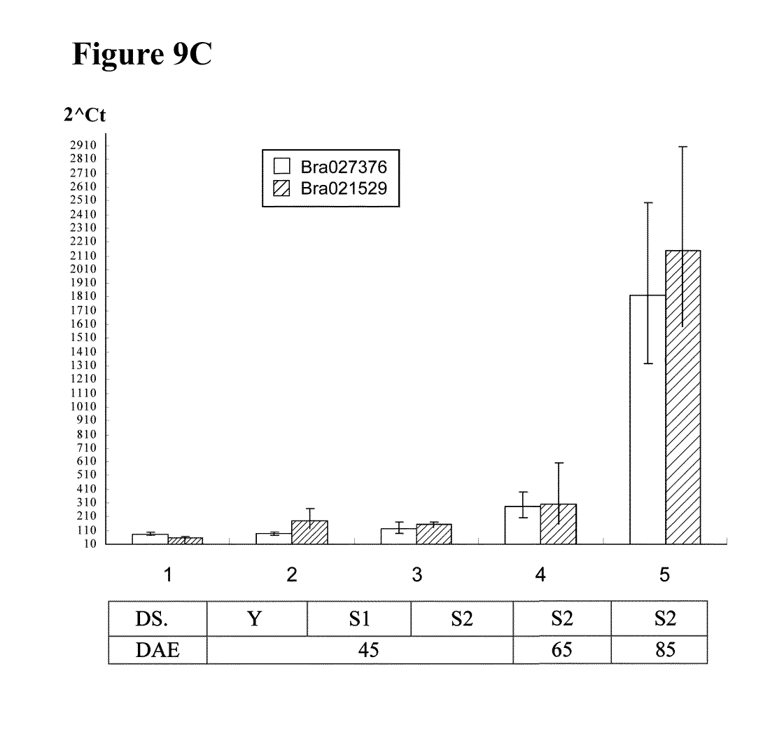

As shown herein, SASP gene expression is highly up-regulated in senescent and cauline leaves (see FIGS. 2 and 9). As described in more detail below, inactivating SASP gene function results in a delay in senescence of photosynthetic tissue. Thus, the plant SASP identified herein is associated with leaf senescence.

The invention relates to methods for making plants with increased yield which may comprise inactivating, repressing or down-regulating the activity of a SASP gene or polypeptide in a plant. Activity of the SASP gene or polypeptide is inactivated, repressed or down-regulated compared to a control plant. The methods comprise molecular biology tools, including mutagenesis (e.g. TILLING) or recombinant DNA technology and do not relate to traditional breeding techniques. According to the methods of the invention, inactivating, repressing or down-regulating the activity of SASP can be achieved through different means. Within the scope of the invention are methods for inhibiting, repressing, inactivating or reducing translation or transcription of the SASP gene, destabilizing SASP transcript stability or SASP polypeptide stability or inhibiting, repressing, inactivating or reducing the activation of the SASP transcript or polypeptide. Thus, the plants described herein have been generated by methods that do not solely rely on traditional breeding methods.

Specifically, in a first aspect, the invention relates to a method for making a transgenic plant with increased yield which may comprise inactivating, repressing or down-regulating the activity of a SASP gene or polypeptide in a plant. In one embodiment, the invention relates to methods for making a SASP knock-out or knock-down plant as described herein.

The term “yield” as described herein relates to yield-related traits. Specifically, these include an increase in biomass and/or seed yield. This can be achieved by increased growth. An increase in yield can be, for example, assessed by the harvest index, i.e. the ratio of seed yield to aboveground dry weight. Thus, according to the invention, yield comprises one or more of: increased seed yield per plant, increased seed filling rate, increased number of filled seeds, increased harvest index, increased number of seed capsules/pods, increased seed size, increased growth or increased branching, for example inflorescences with more branches. Preferably, yield comprises an increased number of seed capsules/pods and/or increased branching. Yield is increased relative to control plants. An increase in yield may be about 5, 10, 20, 30, 40, 50% or more compared to a control plant. Without wishing to be bound by theory, an increase in yield as described herein may for example also be mediated by effects on photosynthetic longevity or on metabolite redistribution.

A control plant as used herein is a plant, for example a wild type plant, which has not been modified according to the methods of the invention. The control plant is typically of the same plant species, preferably the same ecotype as the plant to be assessed. For example, in some of the embodiments described below which describe inactivating, repressing or down-regulating a SASP gene, the control plant is a wild type plant which expresses the endogenous wild type SASP gene.

As used herein, the words “nucleic acid”, “nucleic acid sequence”, “nucleotide”, or “polynucleotide” are intended to include DNA molecules (e.g., cDNA or genomic DNA), RNA molecules (e.g., mRNA), natural occurring, mutated, synthetic DNA or RNA molecules, and analogs of the DNA or RNA generated using nucleotide analogs. It can be single-stranded or double-stranded. Such nucleic acids or polynucleotides include, but are not limited to, coding sequences of structural genes, anti-sense sequences, and non-coding regulatory sequences that do not encode mRNAs or protein products. These terms also encompass a gene. The term “gene” or “gene sequence” is used broadly to refer to a DNA nucleic acid associated with a biological function. Thus, genes may include introns and exons as in genomic sequence, or may comprise only a coding sequence as in cDNAs, and/or may include cDNAs in combination with regulatory sequences.

For the purposes of the invention, “transgenic”, “transgene” or “recombinant” means with regard to, for example, a nucleic acid sequence, an expression cassette, gene construct or a vector which may comprise the nucleic acid sequence or an organism transformed with the nucleic acid sequences, expression cassettes or vectors according to the invention, all those constructions brought about by recombinant methods in which either
(a) the nucleic acid sequences encoding proteins useful in the methods of the invention, or
(b) genetic control sequence(s) which is operably linked with the nucleic acid sequence according to the invention, for example a promoter, or
(c) a) and b)
   are not located in their natural genetic environment or have been modified by recombinant methods, such as mutagenesis, it being possible for the modification to take the form of, for example, a substitution, addition, deletion, inversion or insertion of one or more nucleotide residues. The natural genetic environment is understood as meaning the natural genomic or chromosomal locus in the original plant or the presence in a genomic library. In the case of a genomic library, the natural genetic environment of the nucleic acid sequence is preferably retained, at least in part. The environment flanks the nucleic acid sequence at least on one side and has a sequence length of at least 50 bp, preferably at least 500 bp, especially preferably at least 1000 bp, most preferably at least 5000 bp. A naturally occurring expression cassette—for example the naturally occurring combination of the natural promoter of the nucleic acid sequences with the corresponding nucleic acid sequence encoding a polypeptide useful in the methods of the present invention, as defined above—becomes a transgenic expression cassette when this expression cassette is modified by non-natural, synthetic (“artificial”) methods such as, for example, mutagenic treatment. Suitable methods are described, for example, in U.S. Pat. No. 5,565,350 or WO 00/15815 incorporated herein by reference. Specifically included are modifications of the endogenous locus by mutagenesis, including chemical mutagenesis, leading to a deletion, insertion or substitution in the endogenous locus.

A transgenic plant for the purposes of the invention is thus understood as meaning, as above, that the nucleic acids used in the methods of the invention are not at their natural locus in the genome of said plant, it being possible for the nucleic acids to be expressed homologously or heterologously. However, as mentioned, transgenic also means that, while the nucleic acids according to the different embodiments of the invention are at their natural position in the genome of a plant, the sequence has been modified with regard to the natural sequence, and/or that the regulatory sequences of the natural sequences have been modified. In preferred embodiments described herein, the term transgenic plants includes genetically modified plants wherein the endogenous locus is modified by mutagenesis, including chemical mutagenesis, leading to a deletion, insertion or substitution in the endogenous locus. These plants thus do not carry a transgene to alter expression of the endogenous locus, but the endogenous locus is modified by mutagenesis. The plants have thus been genetically modified and generated by methods that do not solely rely on traditional breeding methods. Transgenic can also be understood as meaning the expression of the nucleic acids according to the invention at an unnatural locus in the genome, i.e. homologous or, preferably, heterologous expression of the nucleic acids takes place.

The reduction, decrease, down-regulation or repression of the activity of the SASP polypeptide or gene according to the methods of the invention is at least 10, 20, 30, 40 or 50% in comparison to the control plant.

In one embodiment, the method comprises making a transgenic plant in which the activity of a SASP polypeptide is inactivated, repressed or down-regulated. In one embodiment, the expression of a gene encoding a SASP polypeptide is inactivated, repressed or down-regulated compared to a control plant. This can be achieved by making a reduction (knock down) or loss of function (knock out) mutant wherein the function of the SASP gene is reduced or lost compared to a wild type control plant. To this end, a mutation is introduced into the SASP gene which disrupts the transcription of the gene leading to a gene product which is not functional or has a reduced function. The mutation may be a deletion, insertion or substitution. A skilled person will know that different approaches can be used to generate such mutants. In one embodiment, insertional mutagenesis is used. In this embodiment, as discussed in the examples, T-DNA may be used as an insertional mutagen which disrupts SASP gene expression. The details of this method are well known to a skilled person. In short, plant transformation by Agrobacterium results in the integration into the nuclear genome of a sequence called T-DNA, which is carried on a bacterial plasmid. The use of T-DNA transformation leads to stable single insertions. Further mutant analysis of the resultant transformed lines is straightforward and each individual insertion line can be rapidly characterized by direct sequencing and analysis of DNA flanking the insertion. Gene expression in the mutant is compared to expression of the SASP gene in a wild type plant and phenotypic analysis is also carried out. Other techniques for insertional mutagenesis include the use of transposons.

In another embodiment, RNA-mediated gene suppression or RNA silencing may be used to achieve silencing of the SASP gene. “Gene silencing” is a term generally used to refer to suppression of expression of a gene via sequence-specific interactions that are mediated by RNA molecules. The degree of reduction may be so as to totally abolish production of the encoded gene product, but more usually the abolition of expression is partial, with some degree of expression remaining The term should not therefore be taken to require complete “silencing” of expression.

Transgenes may be used to suppress endogenous plant genes. This was discovered originally when chalcone synthase transgenes in petunia caused suppression of the endogenous chalcone synthase genes and indicated by easily visible pigmentation changes. Subsequently it has been described how many, if not all plant genes can be “silenced” by transgenes. Gene silencing requires sequence similarity between the transgene and the gene that becomes silenced. This sequence homology may involve promoter regions or coding regions of the silenced target gene. When coding regions are involved, the transgene able to cause gene silencing may have been constructed with a promoter that would transcribe either the sense or the antisense orientation of the coding sequence RNA. It is likely that the various examples of gene silencing involve different mechanisms that are not well understood. In different examples there may be transcriptional or post transcriptional gene silencing and both may be used according to the methods of the invention.

The mechanisms of gene silencing and their application in genetic engineering, which were first discovered in plants in the early 1990s and then shown in Caenorhabditis elegans by Fire and Mello are extensively described in the literature, for example Molnar A et al 2011 incorporated herein by reference.

RNA-mediated gene suppression or RNA silencing according to the methods of the invention includes co-suppression wherein over-expression of the SASP sense RNA or mRNA leads to a reduction in the level of expression of the genes concerned. RNAs of the transgene and homologous endogenous gene are co-ordinately suppressed.

Other techniques used in the methods of the invention include antisense RNA to reduce transcript levels of the endogenous SASP gene in a plant. In this method, RNA silencing does not affect the transcription of a gene locus, but only causes sequence-specific degradation of target mRNAs. An “antisense” nucleic acid sequence comprises a nucleotide sequence that is complementary to a “sense” nucleic acid sequence encoding a SASP protein, or a part of a SASP protein, i.e. complementary to the coding strand of a double-stranded cDNA molecule or complementary to an mRNA transcript sequence. The antisense nucleic acid sequence is preferably complementary to the endogenous SASP gene to be silenced. The complementarity may be located in the “coding region” and/or in the “non-coding region” of a gene. The term “coding region” refers to a region of the nucleotide sequence which may comprise codons that are translated into amino acid residues. The term “non-coding region” refers to 5′ and 3′ sequences that flank the coding region that are transcribed but not translated into amino acids (also referred to as 5′ and 3′ untranslated regions).

Antisense nucleic acid sequences can be designed according to the rules of Watson and Crick base pairing. The antisense nucleic acid sequence may be complementary to the entire SASP nucleic acid sequence, but may also be an oligonucleotide that is antisense to only a part of the nucleic acid sequence (including the mRNA 5′ and 3′ UTR). For example, the antisense oligonucleotide sequence may be complementary to the region surrounding the translation start site of an mRNA transcript encoding a polypeptide. The length of a suitable antisense oligonucleotide sequence is known in the art and may start from about 50, 45, 40, 35, 30, 25, 20, 15 or 10 nucleotides in length or less. An antisense nucleic acid sequence according to the invention may be constructed using chemical synthesis and enzymatic ligation reactions using methods known in the art. For example, an antisense nucleic acid sequence (e.g., an antisense oligonucleotide sequence) may be chemically synthesized using naturally occurring nucleotides or variously modified nucleotides designed to increase the biological stability of the molecules or to increase the physical stability of the duplex formed between the antisense and sense nucleic acid sequences, e.g., phosphorothioate derivatives and acridine-substituted nucleotides may be used. Examples of modified nucleotides that may be used to generate the antisense nucleic acid sequences are well known in the art. The antisense nucleic acid sequence can be produced biologically using an expression vector into which a nucleic acid sequence has been subcloned in an antisense orientation (i.e., RNA transcribed from the inserted nucleic acid will be of an antisense orientation to a target nucleic acid of interest). Preferably, production of antisense nucleic acid sequences in plants occurs by means of a stably integrated nucleic acid construct which may comprise a promoter, an operably linked antisense oligonucleotide, and a terminator.

The nucleic acid molecules used for silencing in the methods of the invention hybridize with or bind to mRNA transcripts and/or insert into genomic DNA encoding a polypeptide to thereby inhibit expression of the protein, e.g., by inhibiting transcription and/or translation. The hybridization can be by conventional nucleotide complementarity to form a stable duplex, or, for example, in the case of an antisense nucleic acid sequence which binds to DNA duplexes, through specific interactions in the major groove of the double helix. Antisense nucleic acid sequences may be introduced into a plant by transformation or direct injection at a specific tissue site. Alternatively, antisense nucleic acid sequences can be modified to target selected cells and then administered systemically. For example, for systemic administration, antisense nucleic acid sequences can be modified such that they specifically bind to receptors or antigens expressed on a selected cell surface, e.g., by linking the antisense nucleic acid sequence to peptides or antibodies which bind to cell surface receptors or antigens. The antisense nucleic acid sequences can also be delivered to cells using vectors.

RNA interference (RNAi) is another post-transcriptional gene-silencing phenomenon which may be used according to the methods of the invention. This is induced by double-stranded RNA in which mRNA that is homologous to the dsRNA is specifically degraded. It refers to the process of sequence-specific post-transcriptional gene silencing mediated by short interfering RNAs (siRNA). The process of RNAi begins when the enzyme, DICER, encounters dsRNA and chops it into pieces called small-interfering RNAs (siRNA). This protein belongs to the RNase III nuclease family. A complex of proteins gathers up these RNA remains and uses their code as a guide to search out and destroy any RNAs in the cell with a matching sequence, such as target mRNA.

Thus, a plant may be transformed to introduce a RNAi, snRNA, dsRNA, siRNA, miRNA, ta-siRNA or cosuppression molecule that has been designed to target the expression of the SASP gene and selectively decreases or inhibits the expression of the gene or stability of its transcript. Preferably, the RNAi, snRNA, dsRNA, siRNA, miRNA, ta-siRNA or cosuppression molecule used in the methods of the invention comprises a fragment of at least 17 nt, preferably 22 to 26 nt and can be designed on the basis of the information shown in SEQ ID No. 1. Guidelines for designing effective siRNAs are known to the skilled person (for example Carmichael et al 2005). Briefly, a short fragment of the target gene sequence (e.g., 19-40 nucleotides in length) is chosen as the target sequence of the siNA of the invention. The short fragment of target gene sequence is a fragment of the target gene mRNA. In preferred embodiments, the criteria for choosing a sequence fragment from the target gene mRNA to be a candidate siRNA molecule include 1) a sequence from the target gene mRNA that is at least 50-100 nucleotides from the 5′ or 3′ end of the native mRNA molecule, 2) a sequence from the target gene mRNA that has a G/C content of between 30% and 70%, most preferably around 50%, 3) a sequence from the target gene mRNA that does not contain repetitive sequences (e.g., AAA, CCC, GGG, TTT, AAAA, CCCC, GGGG, TTTT), 4) a sequence from the target gene mRNA that is accessible in the mRNA, 5) a sequence from the target gene mRNA that is unique to the target gene, 6) avoid regions within 75 bases of a start codon. The sequence fragment from the target gene mRNA may meet one or more of the criteria identified above. The selected gene is introduced as a nucleotide sequence in a prediction program that takes into account all the variables described above for the design of optimal oligonucleotides. This program scans any mRNA nucleotide sequence for regions susceptible to be targeted by siRNAs. The output of this analysis is a score of possible siRNA oligonucleotides. The highest scores are used to design double stranded RNA oligonucleotides that are typically made by chemical synthesis. In addition to siNA which is complementary to the mRNA target region, degenerate siNA sequences may be used to target homologous regions. siNAs according to the invention can be synthesized by any method known in the art. RNAs are preferably chemically synthesized using appropriately protected ribonucleoside phosphoramidites and a conventional DNA/RNA synthesizer. Additionally, siRNAs can be obtained from commercial RNA oligonucleotide synthesis suppliers.

siNA molecules may be double stranded. In one embodiment, double stranded siNA molecules comprise blunt ends. In another embodiment, double stranded siNA molecules comprise overhanging nucleotides (e.g., 1-5 nucleotide overhangs, preferably 2 nucleotide overhangs). In some embodiments, the siRNA is a short hairpin RNA (shRNA); and the two strands of the siRNA molecule may be connected by a linker region (e.g., a nucleotide linker or a non-nucleotide linker). The siNAs of the invention may contain one or more modified nucleotides and/or non-phosphodiester linkages. Chemical modifications well known in the art are capable of increasing stability, availability, and/or cell uptake of the siNA. The skilled person will be aware of other types of chemical modification which may be incorporated into RNA molecules.

In one embodiment, recombinant DNA constructs as described in U.S. Pat. No. 6,635,805, incorporated herein by reference, may be used.

The silencing RNA molecule is introduced into the plant using conventional methods, for example a vector and Agrobacterium mediated transformation. Stably transformed plants are generated and expression of the SASP gene compared to a wild type control plant is analysed.

Silencing of the SASP gene may also be achieved using virus-induced gene silencing.

In another embodiment of the methods of the invention, the transgenic plant is a mutant plant derived from a plant population mutagenised with a mutagen. The mutagen may be fast neutron irradiation or a chemical mutagen, for example selected from the following non-limiting list: ethyl methanesulfonate (EMS), methylmethane sulfonate (MMS), N-ethyl-N-nitrosurea (ENU), triethylmelamine (1′EM), N-methyl-N-nitrosourea (MNU), procarbazine, chlorambucil, cyclophosphamide, diethyl sulfate, acrylamide monomer, melphalan, nitrogen mustard, vincristine, dimethylnitosamine, N-methyl-N′-nitro-Nitrosoguanidine (MNNG), nitrosoguanidine, 2-aminopurine, 7,12 dimethyl-benz(a)anthracene (DMBA), ethylene oxide, hexamethylphosphoramide, bisulfan, diepoxyalkanes (diepoxyoctane (DEO), diepoxybutane (BEB), and the like), 2-methoxy-6-chloro-9[3-(ethyl-2-chloroethyl)aminopropylamino]acridine dihydrochloride (ICR-170) or formaldehyde.

In one embodiment, the method used to create and analyse mutations is targeting induced local lesions in genomes (TLLING), reviewed in Henikoff et al, 2004. In this method, seeds are mutagenised with a chemical mutagen, for example EMS. The resulting M1 plants are self-fertilised and the M2 generation of individuals is used to prepare DNA samples for mutational screening. DNA samples are pooled and arrayed on microtiter plates and subjected to gene specific PCR. The PCR amplification products may be screened for mutations in the SASP target gene using any method that identifies heteroduplexes between wild type and mutant genes. For example, but not limited to, denaturing high pressure liquid chromatography (dHPLC), constant denaturant capillary electrophoresis (CDCE), temperature gradient capillary electrophoresis (TGCE), or by fragmentation using chemical cleavage. Preferably the PCR amplification products are incubated with an endonuclease that preferentially cleaves mismatches in heteroduplexes between wild type and mutant sequences. Cleavage products are electrophoresed using an automated sequencing gel apparatus, and gel images are analyzed with the aid of a standard commercial image-processing program. Any primer specific to the SASP gene may be utilized to amplify the SASP genes within the pooled DNA sample. Preferably, the primer is designed to amplify the regions of the SASP gene where useful mutations are most likely to arise, specifically in the areas of the SASP gene that are highly conserved and/or confer activity. To facilitate detection of PCR products on a gel, the PCR primer may be labelled using any conventional labelling method.

Rapid high-throughput screening procedures thus allow the analysis of amplification products for identifying a mutation conferring the reduction or inactivation of the expression of the SASP gene as compared to a corresponding non-mutagenised wild type plant. Once a mutation is identified in a gene of interest, the seeds of the M2 plant carrying that mutation are grown into adult M3 plants and screened for the phenotypic characteristics associated with the SASP gene. Loss of and reduced function mutants with increased yield and decrease or no expression during leaf senescence compared to a wild type control can thus be identified.

In another embodiment of the methods of the invention, inactivating, repressing or down-regulating the activity of SASP can be achieved by manipulating the expression of SASP inhibitors in a transgenic plant. For example, a gene expressing a protein that inhibits the expression of the SASP gene or activity of the SASP protein can be introduced into a plant and over-expressed. The inhibitor may interact with the regulatory sequences that direct SASP gene expression to down-regulate or repress SASP gene expression. For example, the inhibitor may be a transcriptional repressor. Alternatively, it may interact and repress transcriptional regulators, for example transcription factors, that positively regulate expression of the SASP gene. Alternatively, the inhibitor it may directly interact with the SASP protein to inhibit its activity or interact with modulators of the SASP protein. For example, the activity of the SASP protein may be inactivated, repressed or down-regulated by manipulating post-transcriptional modifications, for example glycosylation, of the SASP protein resulting in a reduced or lost activity.

In one embodiment, the methods of the invention comprise comparing the activity of the SASP polypeptide and/or expression of the SASP gene with the activity of the SASP polypeptide and/or expression of the SASP gene in a control plant.

In one embodiment of the methods described herein, the method may include a further step manipulating the activity of a second plant gene. This may, for example be a SASP homologue.

Further, in another embodiment, the present invention relates to a plant, plant tissue, harvested plant material or propagation material of a plant obtained or obtainable by the methods described herein.

In another embodiment, the invention relates to a method for increasing yield which may comprise inactivating, repressing or down-regulating the activity of SASP polypeptide. The method may comprise making a transgenic plant following conventional protocols described in the literature cited herein.

In another aspect, the invention relates to a method for delaying senescence which may comprise inactivating, repressing or down-regulating the activity of SASP polypeptide. In another aspect, the invention relates to a method for making a transgenic plant with delayed senescence which may comprise inactivating, repressing or down-regulating the activity of a SASP polypeptide in a plant. The details of this method are discussed above.

According to the preferred methods and plants of the invention, the SASP polypeptide is encoded by a SASP gene that comprises or consists of a sequence as shown in SEQ ID No 1, a homologue, paralogue, orthologue, allelic variant or functional variant thereof. Accordingly, the SASP polypeptide comprises or consists of a sequence as shown in SEQ ID No 1, a homologue, paralogue or orthologue thereof. The orthologue may be selected from any plant species and examples of preferred plants are given below. For example, the orthologue may be a brassica, wheat, rice or maize orthologue.

Thus, the SASP gene according to the different aspects of the invention, including the plants and methods described herein, comprises or consists of a sequence as shown in SEQ ID No 1, a homologue, paralogue, orthologue, allelic variant or functional variant thereof. In one embodiment, the SASP gene comprises or consists of a sequence as shown in SEQ ID No 3, 4, 5 or 7. In one embodiment, the SASP gene comprises or consists of a sequence as shown in SEQ ID No 9. SASP polypeptides according to the invention comprise or consist of the corresponding peptides, e.g. as shown in SEQ ID No. 2, 6, 8.

Orthologues and paralogues encompass evolutionary concepts used to describe the ancestral relationships of genes. Paralogues are genes within the same species that have originated through duplication of an ancestral gene; orthologues are genes from different organisms that have originated through speciation, and are also derived from a common ancestral gene.

As shown in the examples, the AtSASP gene was knocked out in Arabidopsis. The inventors have shown that knock out plant shows increased yield and delayed senescence. However, the skilled person would know that a homologue, paralogue, orthologue of AtSASP gene can be inactivated according to the methods of the invention in any monocot or dicot plant as further defined below.

Homologues and orthologues of AtSASP as shown in SEQ ID No. 1 or the polypeptide sequence as shown SEQ ID No. 2 can be derived from any plant as long as the homologue confers the herein mentioned activity of increasing yield, i.e. it is a functional equivalent of said molecules. Non-limiting examples of homologues and orthologues of AtSASP are provided herein. The homologue of the AtSASP gene or polypeptide shown in SEQ ID No. 1 or 2 has, in increasing order of preference, at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% overall sequence identity to the nucleic acid or amino acid represented by SEQ ID NO: 1 or 2. The overall sequence identity is determined using a global alignment algorithm, such as the Needleman Wunsch algorithm in the program GAP (GCG Wisconsin Package, Accelrys), preferably with default parameters and preferably with sequences of mature proteins (i.e. without taking into account secretion signals or transit peptides).

The plant according to the different aspects of the invention may be a dicot plant which may be selected from the families including, but not limited to Asteraceae, Brassicaceae (e.g. Brassica napus), Chenopodiaceae, Cucurbitaceae, Leguminosae (Caesalpiniaceae, Aesalpiniaceae Mimosaceae, Papilionaceae or Fabaceae), Malvaceae, Rosaceae or Solanaceae. For example, the plant may be selected from lettuce, sunflower, Arabidopsis, spinach, water melon, squash, cabbage, broccoli, tomato, potato, capsicum, tobacco, cotton, okra, apple, rose, strawberry, alfalfa, bean, soybean, field (fava) bean, pea, lentil, peanut, chickpea, coffee, cocoa, alfalfa, apricots, apples, pears, peach, grape vine or citrus species. In one embodiment, the plant is oilseed rape.

Also included are biofuel and bioenergy crops such as sugar cane, oilseed rape/oil-seed rape, linseed, jatropha, oil-palm, copra and willow, eucalyptus, poplar, poplar hybrids, switchgrass, Miscanthus or gymnosperms, such as loblolly pine. Also included are crops for silage (e.g. forage grass species or forage maize), grazing or fodder (pasture grasses, clover, sanfoin, alfalfa), fibres (e.g. cotton, flax), building materials (e.g. pine, oak), pulping (e.g. poplar), feeder stocks for the chemical industry (e.g. high erucic acid oil seed rape, linseed), rubber plants, and crops for amenity purposes (e.g. turf grasses for sports and amenity surfaces), ornamentals for public and private gardens (e.g. species of Angelonia, Begonia, Catharanthus, Euphorbia, Gazania, Impatiens, Nicotiana, Pelargonium, Petunia, Rosa, Verbena, and Viola) and flowers of any plants for the cut-flower market (such as tulips, roses, daffodils, lilies, stallions, gerbera, carnations, chrysanthemums, irises, gladioli, alstromerias, marigold, sweet pea, freesia, anemone poppy).

A monocot plant may, for example, be selected from the families Arecaceae, Amaryllidaceae or Poaceae. For example, the plant may be a cereal crop, such as wheat, rice, barley, maize, oat, sorghum, rye, onion, leek, millet, buckwheat, turf grass, Italian rye grass, switchgrass, Miscanthus, sugarcane or Festuca species.

Preferably, the plant is a crop plant. By crop plant is meant any plant which is grown on a commercial scale for human or animal consumption or use for other non-food/feed use. Preferred plants are maize, wheat, Durum wheat, rice, oilseed rape (or canola), sorghum, sugar cane, soybean, potato, tomato, barley, rye, oats, pea, bean, field bean, sugar beet, oil-palm, groundnut, peanut, cassava, copra, raisin, coffee, cotton, lettuce, banana, broccoli or other vegetable brassicas, jatropha, eucalyptus or poplar.

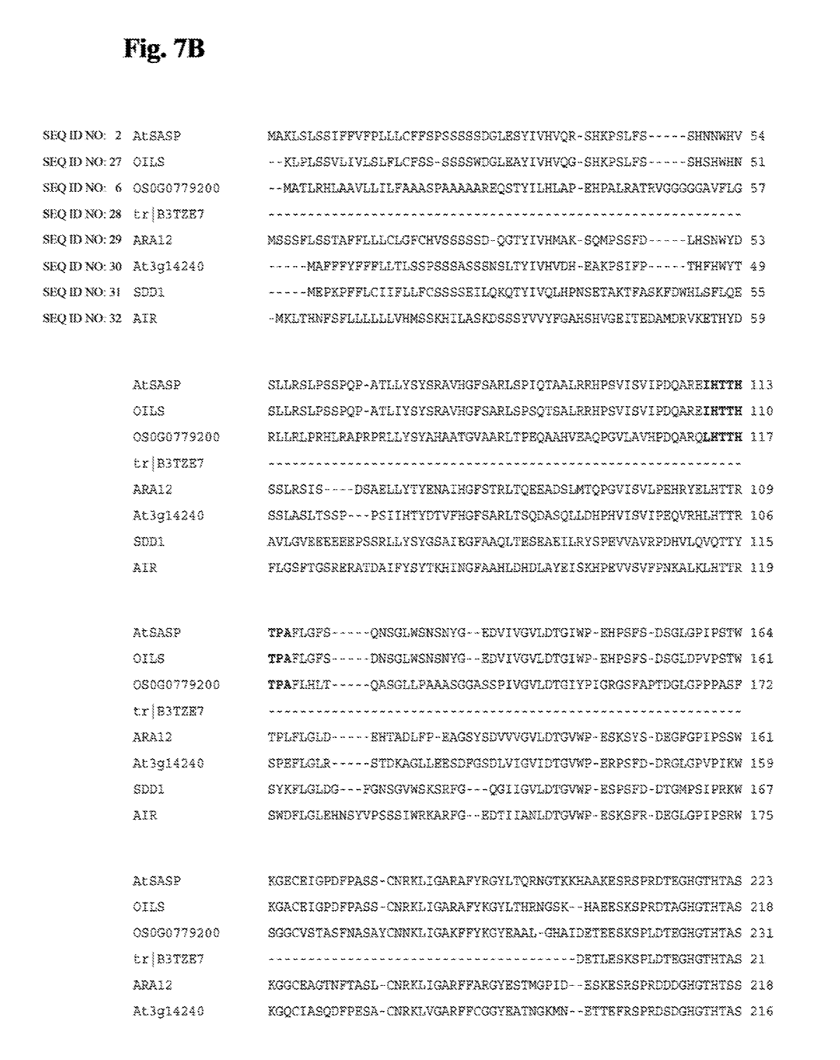

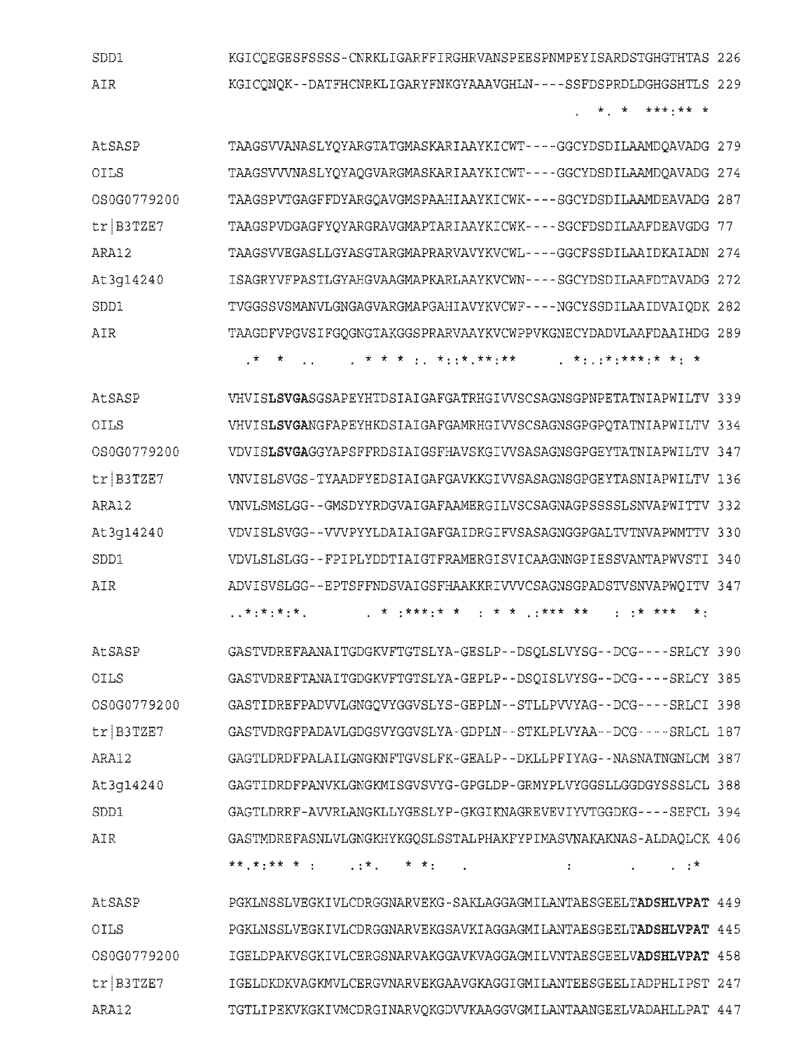

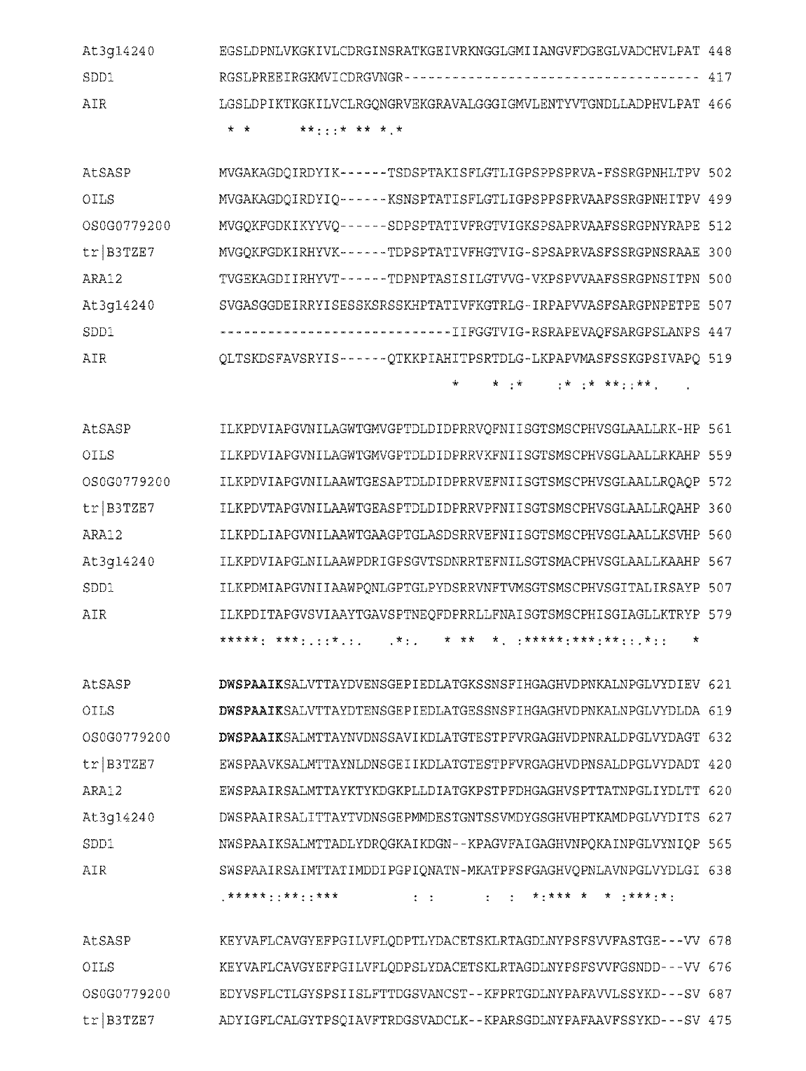

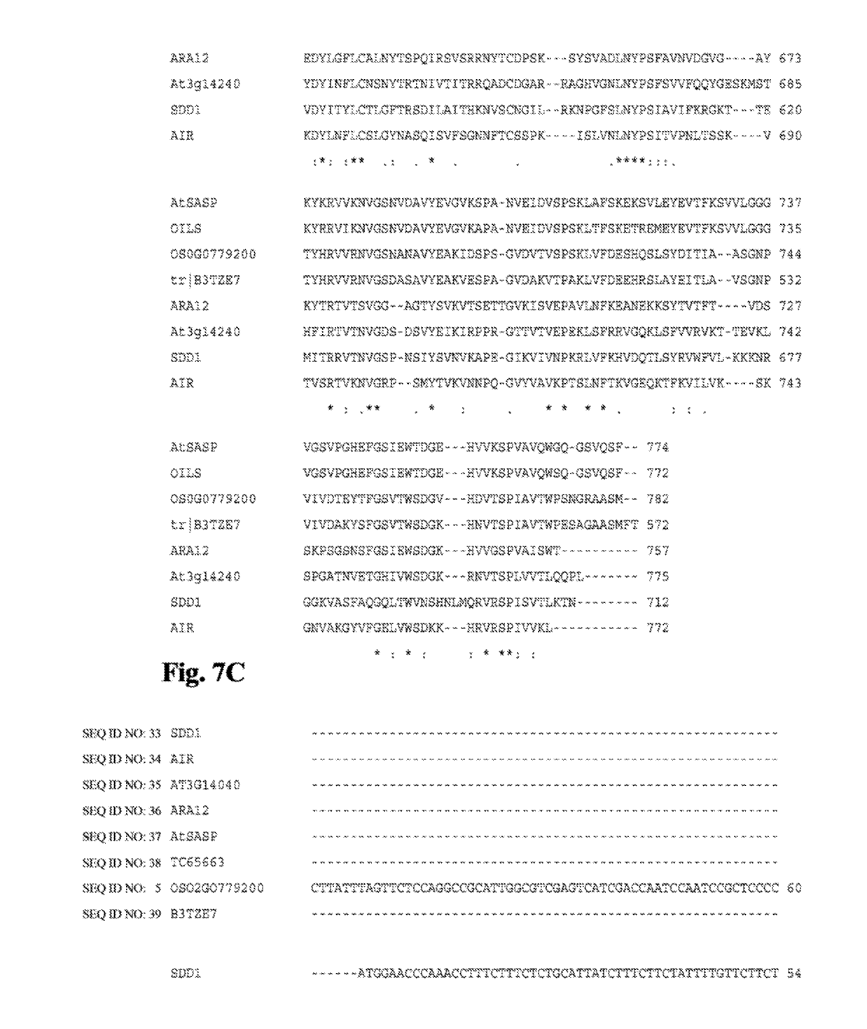

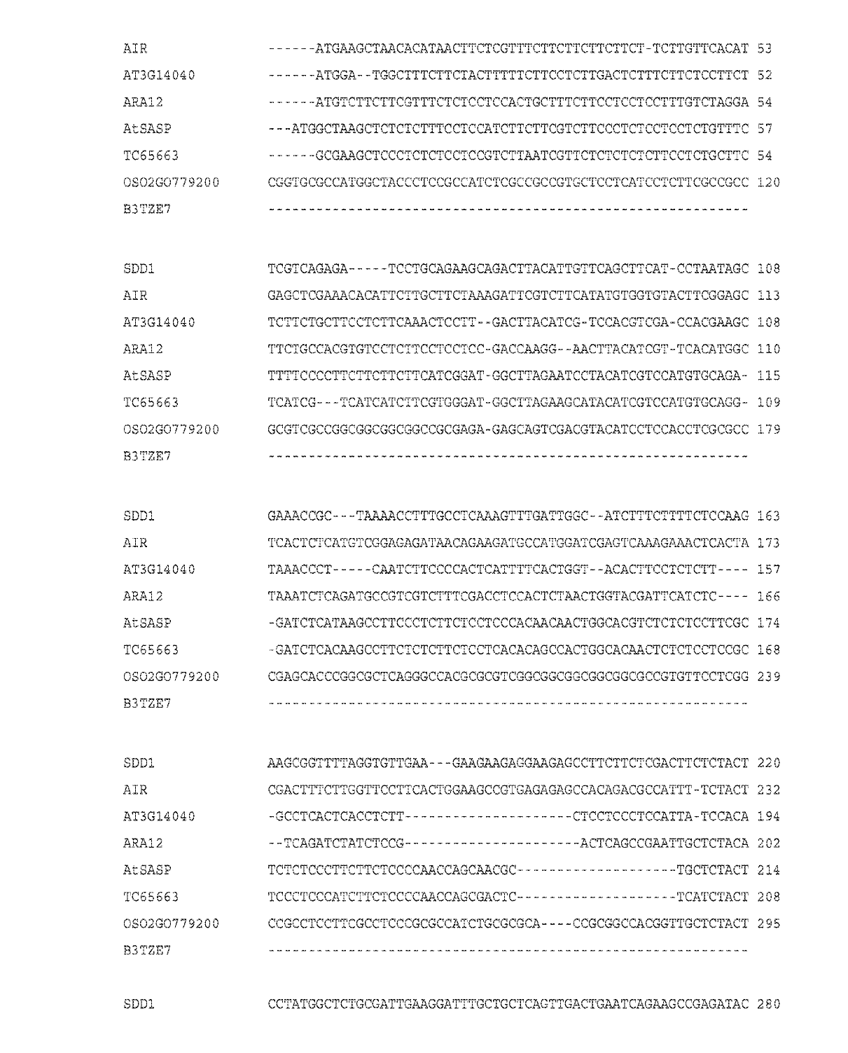

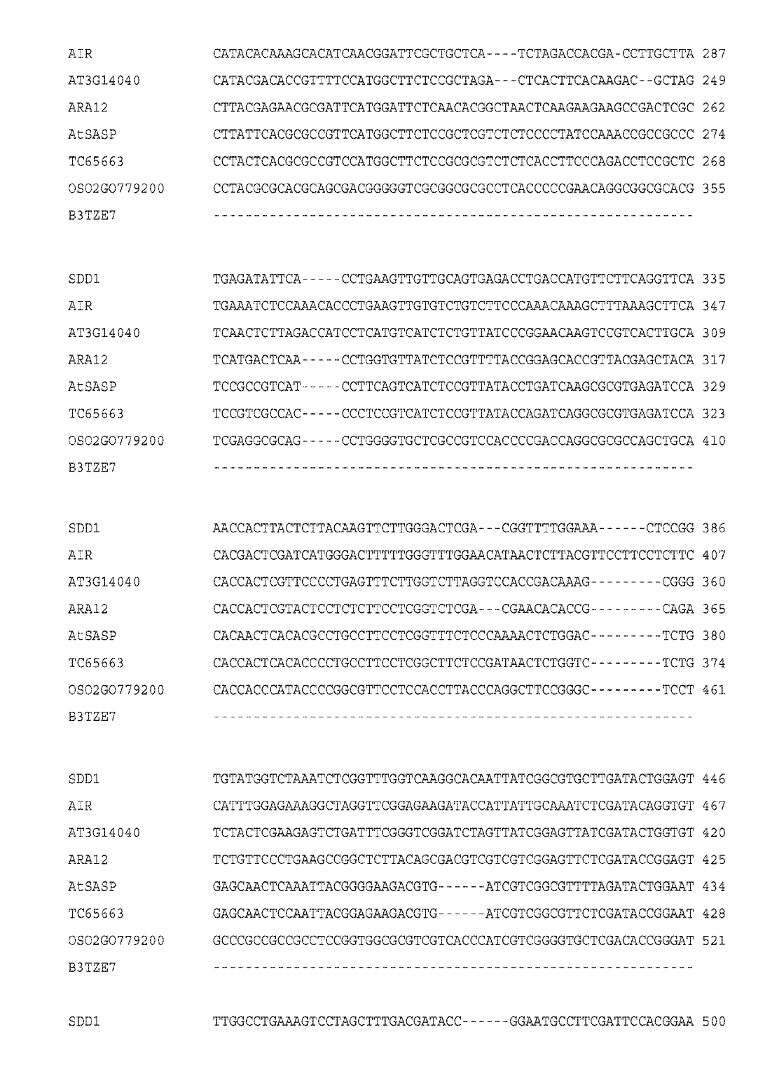

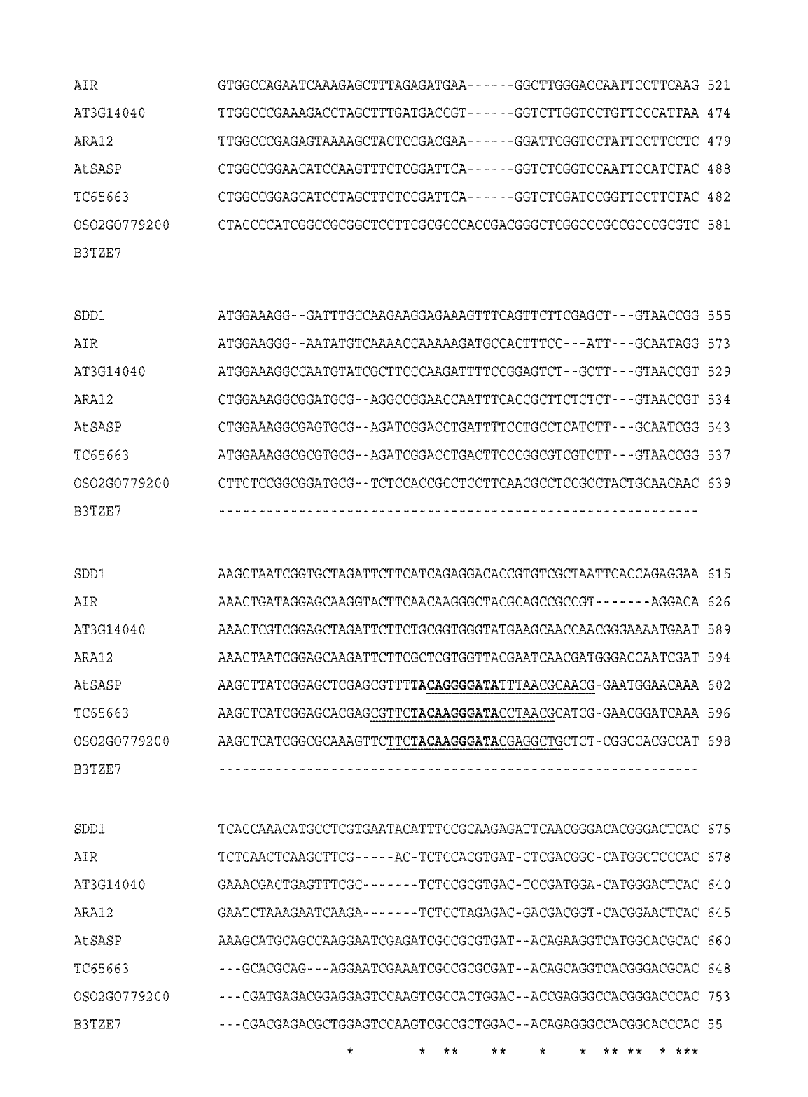

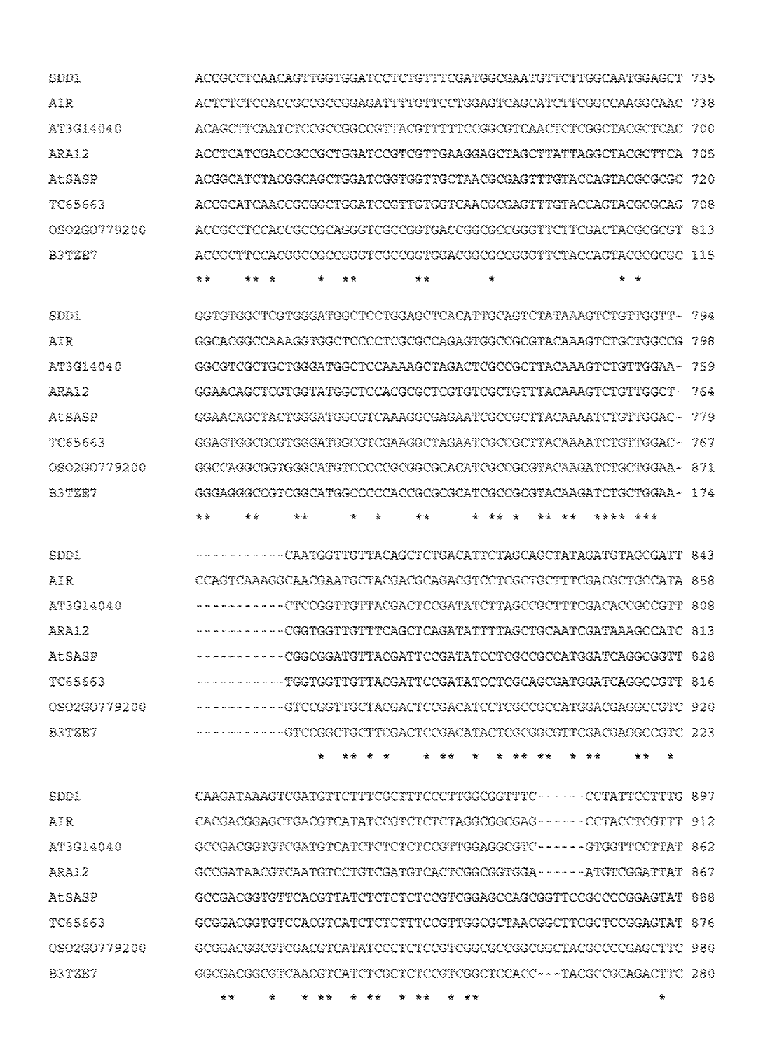

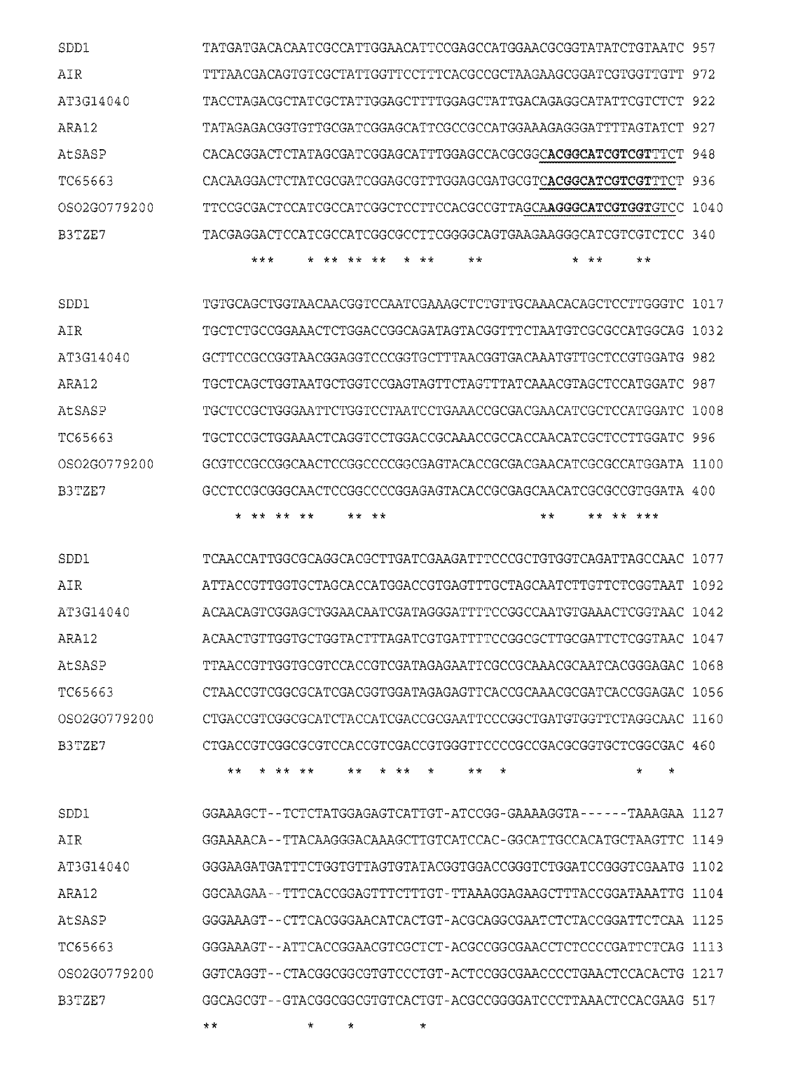

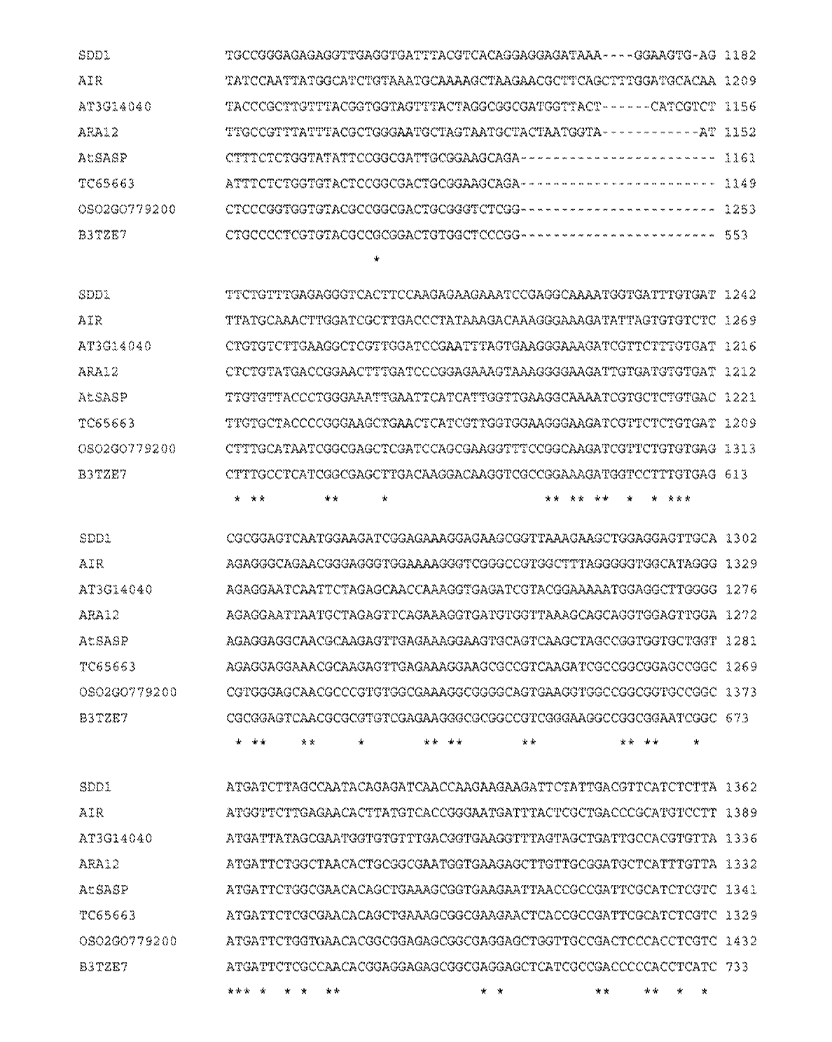

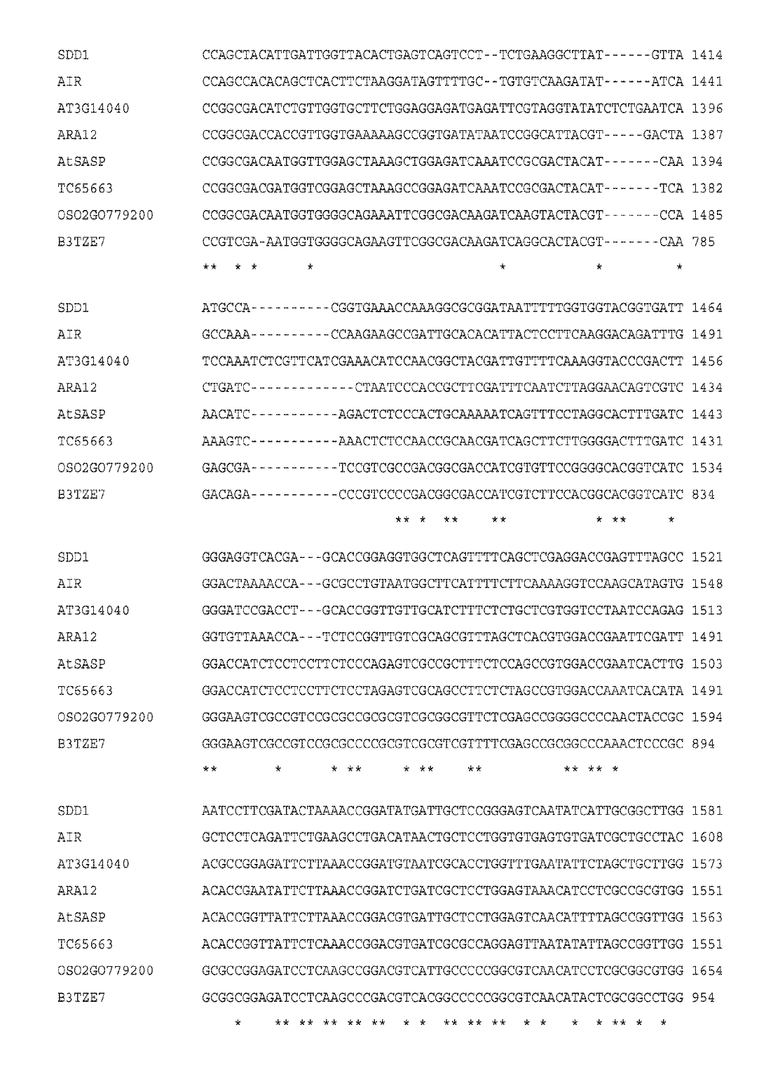

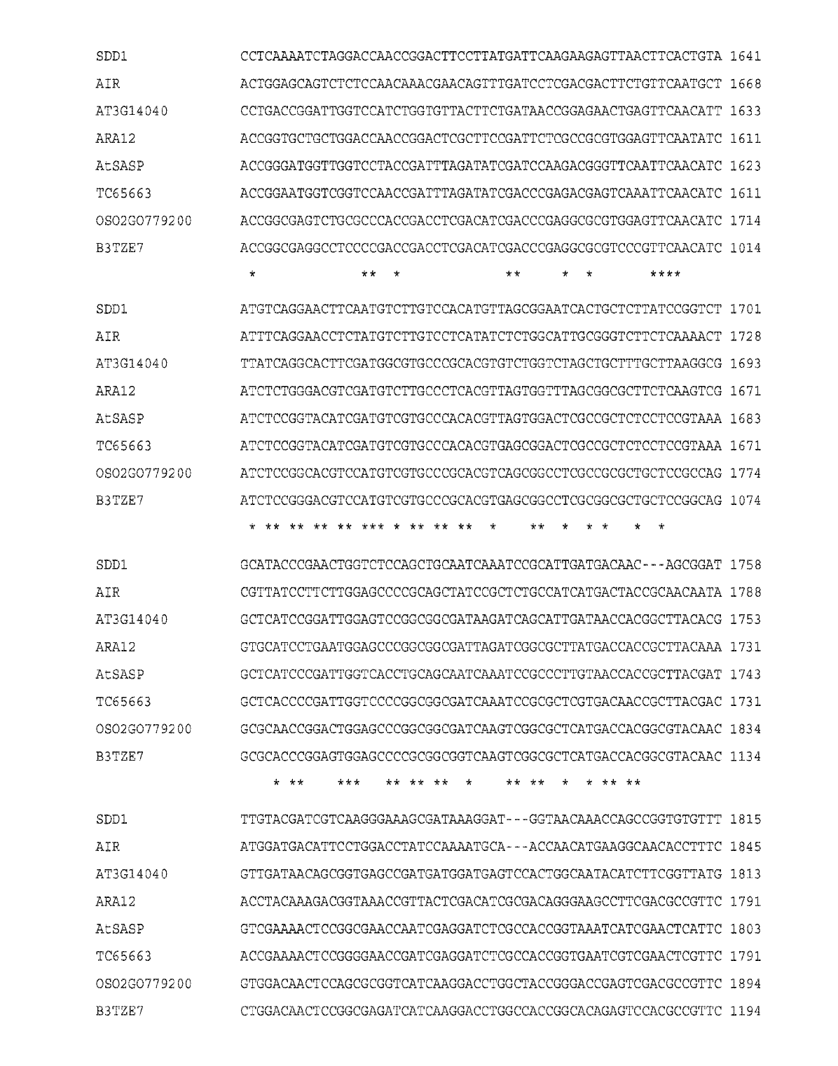

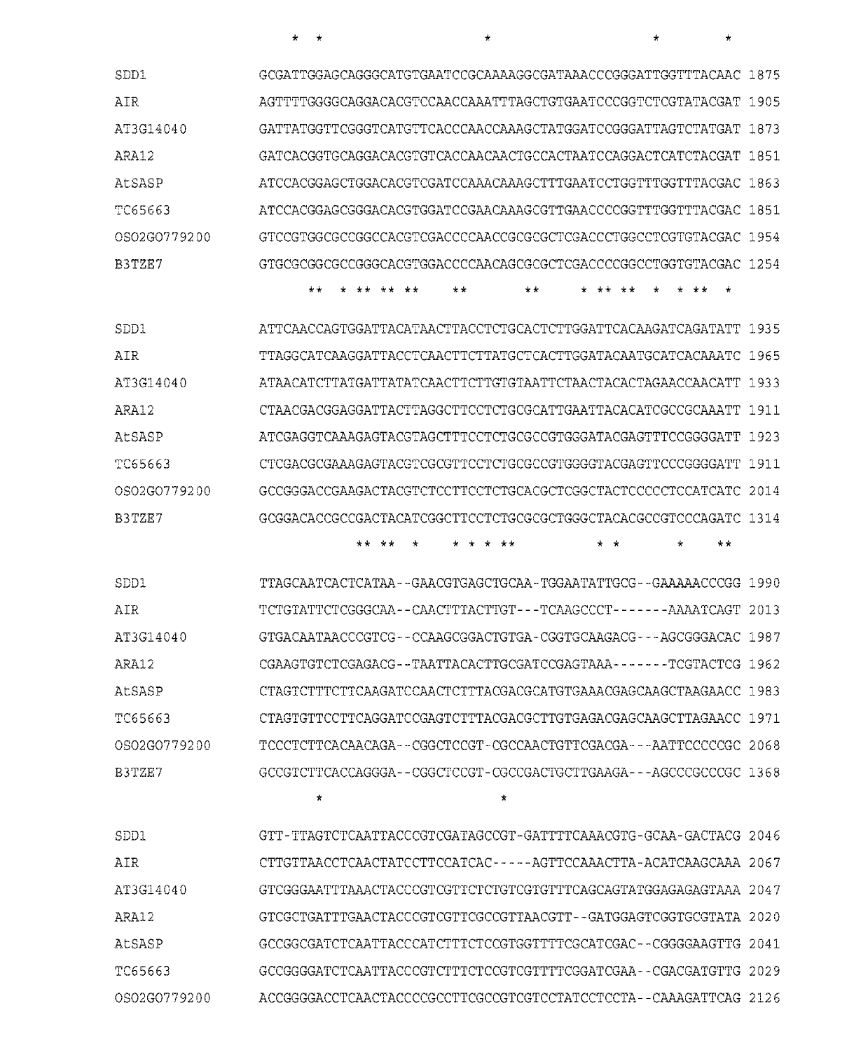

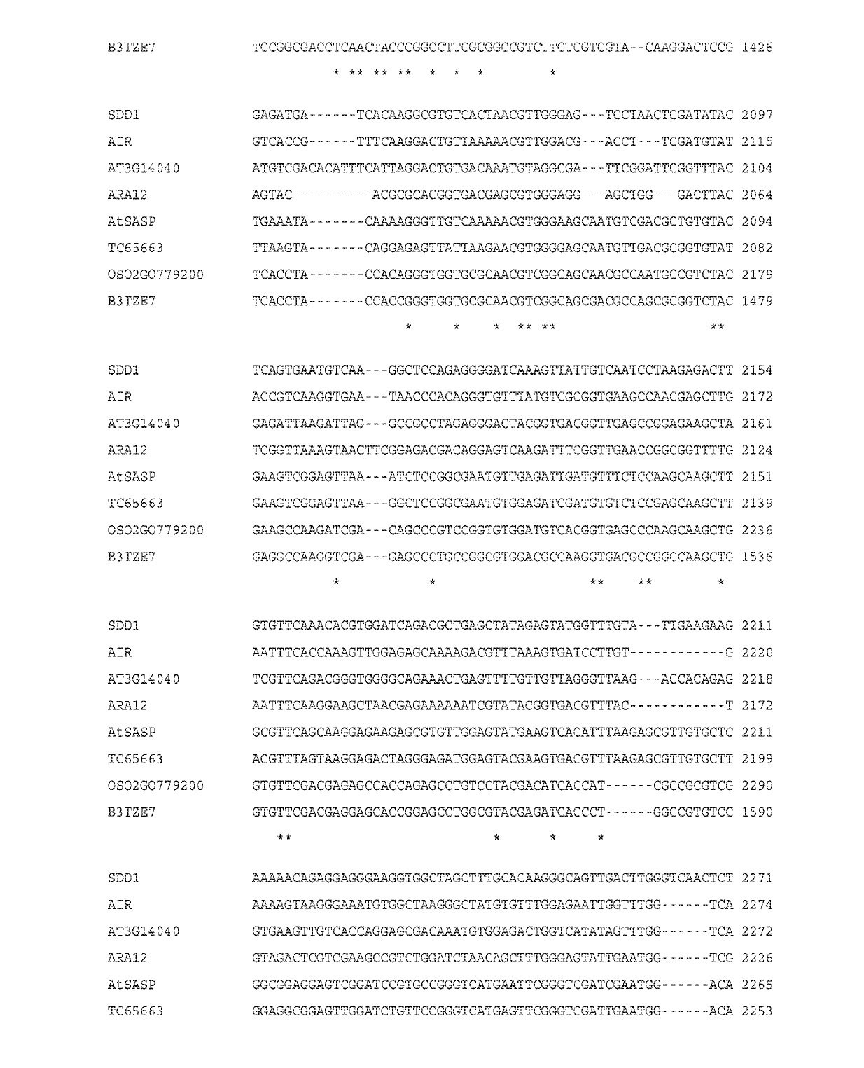

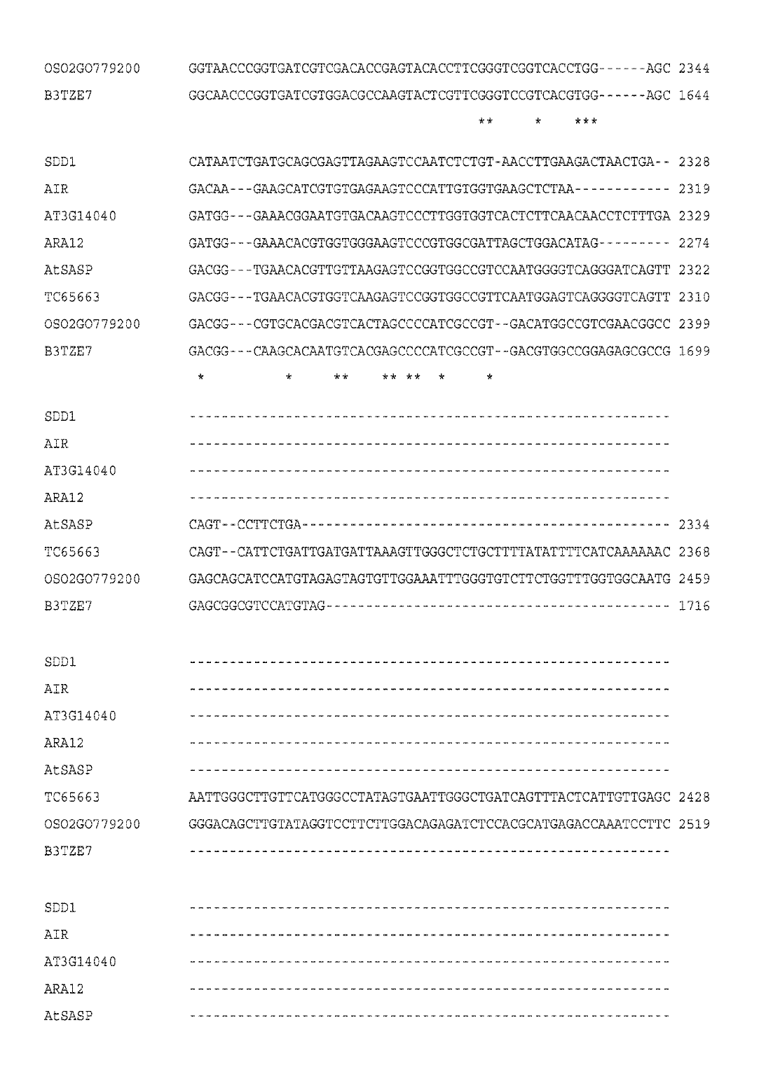

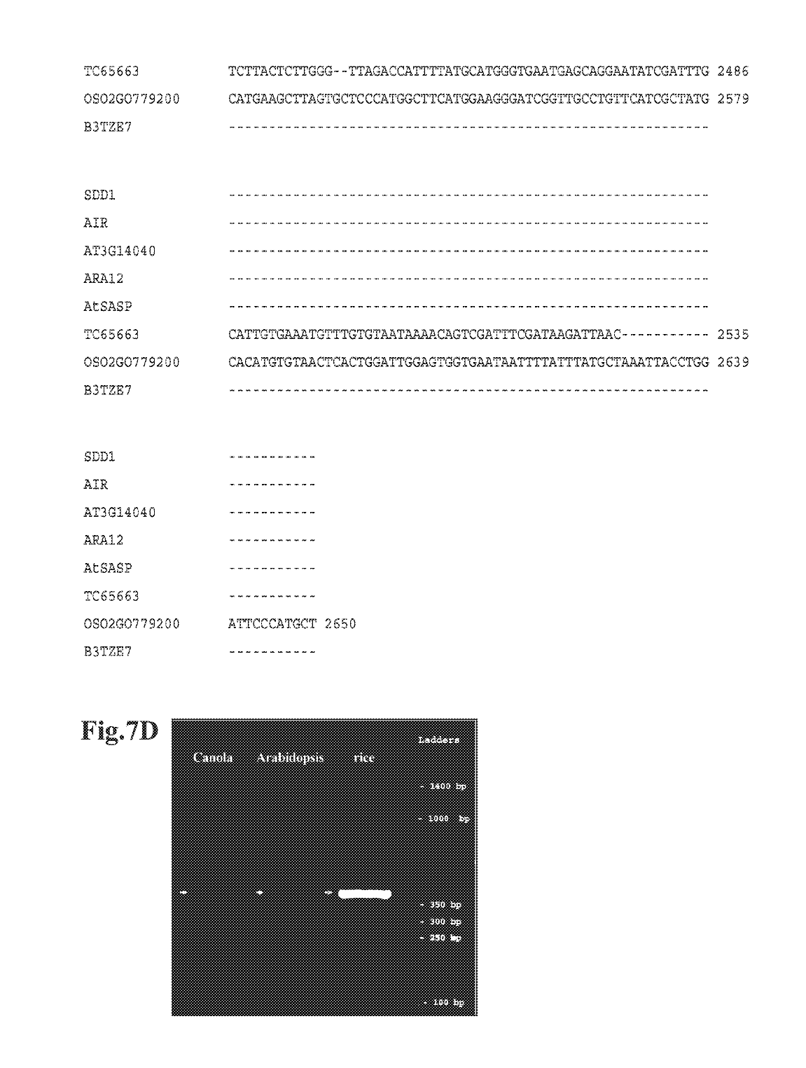

Homologues or orthologues can be identified in other species using bioinformatics. The function of the identified homologous or orthologous gene can be easily tested by routine methods, for example by using the identified gene to complement the Arabidopsis loss of function phenotype (AtSASP-KO) or by creating reduction or loss of function mutants of said endogenous homologous or orthologous gene in the plant species in which it is found. Primers can be designed based on these conserved regions (FIG. 7C underlined letters) to allow PCR amplification of partial sequences of SASP putative orthologues in other species as shown in the examples and figures.

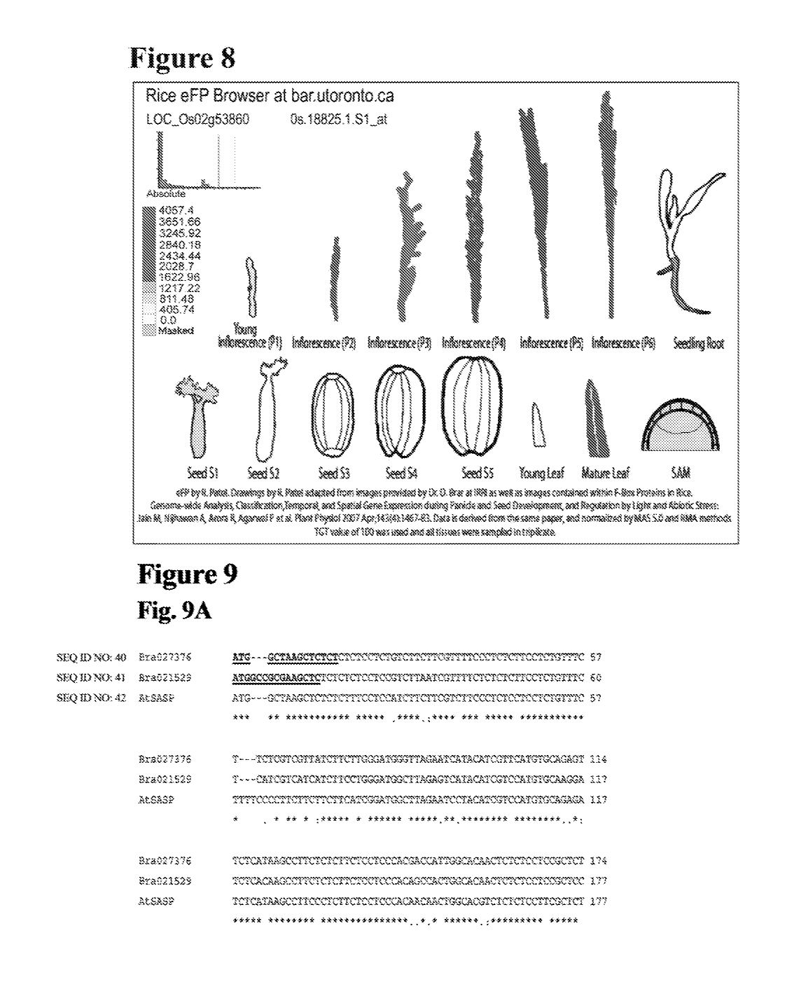

Specifically, as shown in the examples, BLAST searching and multiple sequence alignment has identified orthologues of AtSASP in oil-seed rape (Brassica napus), rice, wheat (FIG. 7) and maize. The subtilases AtSASP, T65663 (an oil-seed rape SASP), Os02g0779200 (a rice SASP) and B3TZE7 (a wheat SASP) share conserved domains that are not present in the Arabidopsis subtilases SDD1, ARA12 and At3g14240 (FIGS. 7B and 7C, bold letters, in the amino acid and nucleic acid sequences, respectively). In silico expression analysis showed upregulation of Os02g0779200 in mature leaves, similar to the expression of AtSASP (FIG. 8). B3TZE7 was identified as a gene that is upregulated in senescing leaves in Triticum aestivum (Arroz et al., 2008 database entry on uniprot: http://www.uniprotorg/uniprot/B3TZE7) and shows high similarity to AtSASP (see examples and figures).

In one embodiment of the different aspects of the invention, the polypeptide homologues or orthologues comprise motifs and a domain structure characteristic for plant subtilase proteases (a signal sequence, a protease associated domain, a “subtilisin/peptidase domain”, a catalytic triad of amino acids D, H, S (Beers et al., 2003, Rose et al., 2010), and other non-exclusive but highly conserved domains, i.e. SDILAA (SEQ ID No. 10), SGTSMSCPHVSG (SEQ ID No. 11), GAGHV (SEQ ID No. 12)) and the following domains which are substantially identical to the following domains identified in AtSASP: IHTTHTPA (SEQ ID No. 13), LSVGA (SEQ ID No. 14) and ADSHLVPAT (SEQ ID No. 15) (FIG. 7).

The inventors have also analysed in silico expression of 56 Arabidopsis subtilisin protease genes using efpBrowser software and have identified four genes homologous to AtSASP for which the expression is associated with senescence, similar to expression of the gene shown in SEQ ID No. 1: At1g32960, At1g32950, At1g32940 and At5g19860. These are within the scope of the invention.

In another aspect, the invention relates to an isolated nucleic acid sequence which may comprise or consist of a sequence as shown in SEQ ID No. 1 or an isolated polypeptide as shown in SEQ OD No. 2, a functional part, variant, homologue or orthologue thereof. Orthologues may for example include gene SEQ ID No. 3, 4, 5, 7 or 9 or equivalent peptide sequences. In one embodiment, the wheat orthologue B3TZE7 is specifically disclaimed.

The term “functional part or functional variant of SASP” as used herein refers to a variant gene sequence or part of the gene sequence which retains the biological function of the full non-variant sequence described herein.

In another aspect, the invention relates to an expression cassette which may comprise an isolated nucleic acid sequence which may comprise or consist of a sequence as shown in SEQ ID No. 1 a functional part, variant, homologue or orthologue thereof operably linked to a regulatory element. The regulatory element may be a promoter. The invention also relates to a vector which may comprise such expression cassette.

In another aspect, the invention relates to a transgenic plant cell, plant or a part thereof wherein the activity of a SASP polypeptide encoded by a SASP gene is inactivated, repressed or down-regulated. This plant shows increased yield. For example, the plant may be a knock out or knock down mutant plant in which the expression of the SASP gene is abolished or reduced. In another embodiment, the stability of the SASP transcript is decreased using silencing technology. The transgenic plant cell, a plant or a part thereof may be derived from a monocotyledonous or dicotyledonous plant as described herein. Preferably, the plant is selected from maize, wheat, Durum wheat, rice, oilseed rape (or canola), sorghum, sugar cane, soybean, potato, tomato, barley, rye, oats, pea, bean, field bean, sugar beet, oil-palm, groundnut, peanut, cassava, copra, raisin, coffee, cotton, lettuce, banana, broccoli or other vegetable brassicas, jatropha, eucalyptus or poplar. The invention also relates to a plant tissue, plant, harvested plant material or propagation material of a plant which may comprise the transgenic plant cell. The terms SASP gene and SASP polypeptide are as defined herein and encompasses a nucleic acid sequence which may comprise or consist of a sequence as shown in SEQ ID No. 1 a functional part, variant, homologue or orthologue thereof as defined herein. Polypeptide homologues or orthologues comprise motifs characteristic for plant subtilase proteases and the conserved domains: IHTTHTPA, LSVGA and ADSHLVPAT (FIG. 7). Orthologues may for example include gene SEQ ID No. 3, 4, 5, 7 or 9 or equivalent peptide sequences.

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### EXAMPLES

The invention is further described in the non-limiting examples.

### 1. SASP Detection and Identification

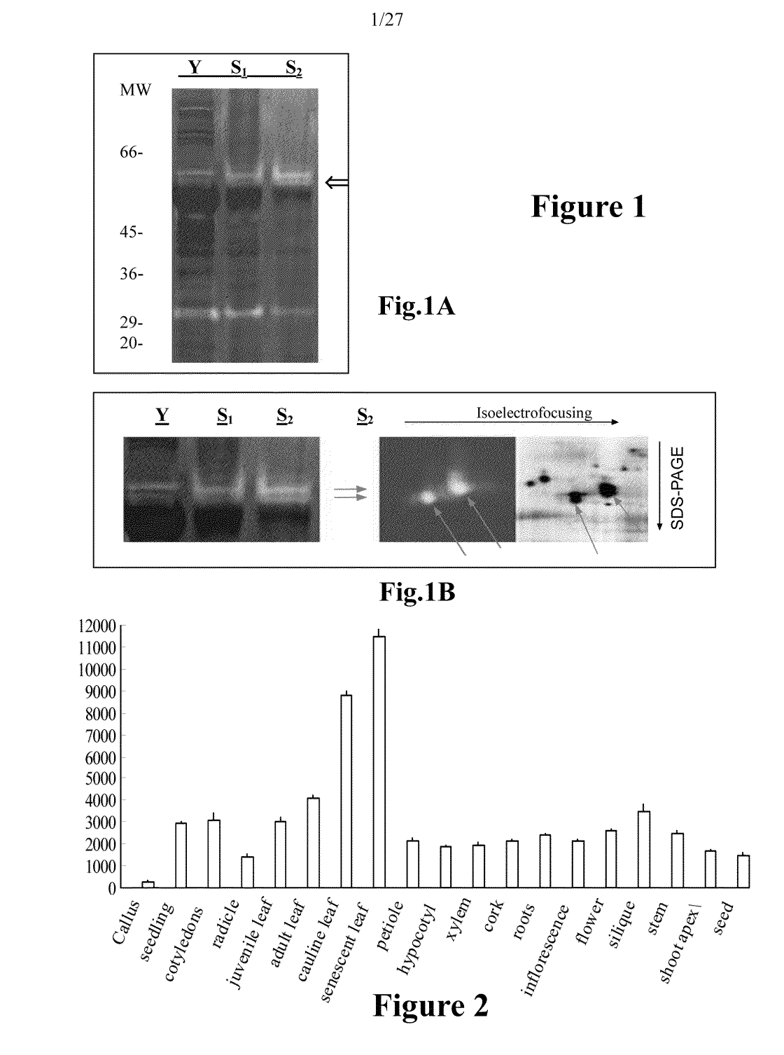

SASP was first identified in Arabidopsis in a proteomic study designed to identify proteases with increased activity during leaf senescence. The approach combined zymograms (in gel proteolytic activity assays) and protein separation in 2D gels followed by mass spectrometric identification of the proteins of interest. Zymograms revealed two major bands of proteolytic activity that appeared in extracts of young leaves and whose intensity increased along leaf development, with the most intense activity during leaf senescence (FIG. 1A). In order to purify the proteases responsible for the two bands of activity, leaf proteins extracted from senescing leaves were separated in 2D zymograms and 2D conventional gels (FIG. 1B). Mass spectrometry analysis identified the two proteins, corresponding both to the same subtilisin type serine protease, gi 22331076, Gene ID 820621, At3g14067 locus in the Arabidopsis genome. Due to its activity profile, we named At3g14067 as SASP (Senescence Associated Subtilisin Protease). SASP possesses a nominal mass of 82.4 kDa and according to zymograms the active protease has at least two isoforms of 59 and 61 kDa, that correspond to the typical mature size of subtilisin proteases (Berger and Altmann, 2000, Beers et al., 2003). The occurrence of two isoforms of this subtilisin protease could be due to posttranslational modifications, i.e., probable glycosylations as SASP sequence has four potential glycosylation sites (www.expasy.ch). According to DNA microarrays analysis with the Genvestigator database and software (Zimmermann et al., 2004) At3g14067 expression is highly (about 4-5 fold) up-regulated during leaf senescence and also, to a slightly lesser extent, in cauline leaves compared with a much lower expression level in other tissues of the plant (FIG. 2).

In vitro proteolytic activity was assayed in zymograms (SDS-PAGE activity gels) containing gelatin as a substrate for proteases (Martinez et al., 2007). Proteolytic activity of leaf extracts from young, mature and senescing leaves was compared in 1D SDS-PAGE activity gels. For protease purification, leaf extracts from senescing leaves were analyzed in 2D gels.

Materials and Methods

Sample preparation: For 1D zymograms leaves were homogenized in 50 mM Tris pH 7.5, 20 mM Cysteine, 1% PVP (Polyvinylpyrrolidone, insoluble), and centrifuged at 12000 g and 4° C. for 15 min. Sample buffer (Laemmli) was added to the sample right before running the electrophoresis. For 2D gels, leaf extracts were solubilized in 25 mM Tris pH 7.5, 20 mM Cysteine and passed through a 10 kDa cut off column (Microcon®, Millipore), proteins were recovered in 250 μl water, 2% Triton X-100, 15 mM DTT, 2M urea, 20 μM leupeptin and 0.2% ampholytes (pH 4-7, Bio-Rad), and cleared by centrifugation at 90000 g for 20 min at 4° C.

Electrophoresis and proteolytic activity: Isoelectrofocusing was performed in immobilized pH gradient strips (IPG) in the pH ranges 3-10 and 4-7 in a BioRad IEF Cell using the following program: 50 V-4 h, 100V-1 h, 200 V-1 h, and 10000 V until 60000 Vh. Then the strips were equilibrated in Laemmli buffer before running in the second dimension (SDS-PAGE gels). SDS-PAGE gels were made of 12% w/v acrylamide with or without 0.04% w/v gelatin and run at 4° C.

After electrophoresis, activity gels (gels containing gelatin) were washed in 2% Triton X-100 and incubated in 80 mM AcNa pH 5.5, 20 mM DTT at 37° C. overnight. Activity gels were stained with Coomassie Blue whereas conventional gels (without gelatin) were silver stained (Schevchenko et al., 1996). Proteolytic activity develops in activity gels as white bands or spots (1D or 2D gel, respectively) in a Coomassie Blue background. The proteases responsible for senescence-associated proteolytic activity were detected in the silver stained gel by superimposing 2D silver stained and 2D activity gels.

Mass spectrometry analysis and identification of the detected proteases: The spots corresponding to the proteases of interest were cut off from the silver stained gel and trypsin digested (Trypsin Gold, Promega). The resulting peptides were washed several times with 5% formic acid and 80% acetonitrile, concentrated and desalted as in Zhang et al. (2004). Samples were analyzed in a LC-ESI Ms/MS (API QSTAR) Applied Biosystems Nano Electrospray Protana Toronto.

Protein identification was performed with the Mascot software and the National Center of Biotechnology Information (NCBI) database. Errors<100 ppm were omitted.

SASP Mutant “Knock Out” Generation and Phenotypic Analysis

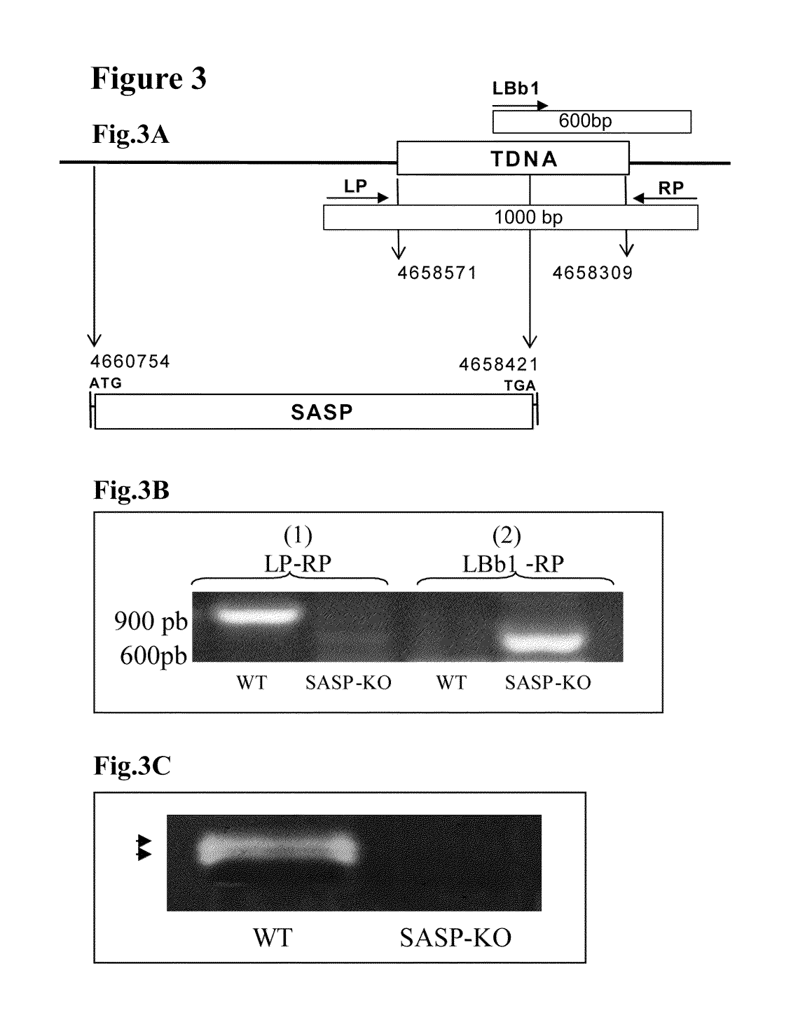

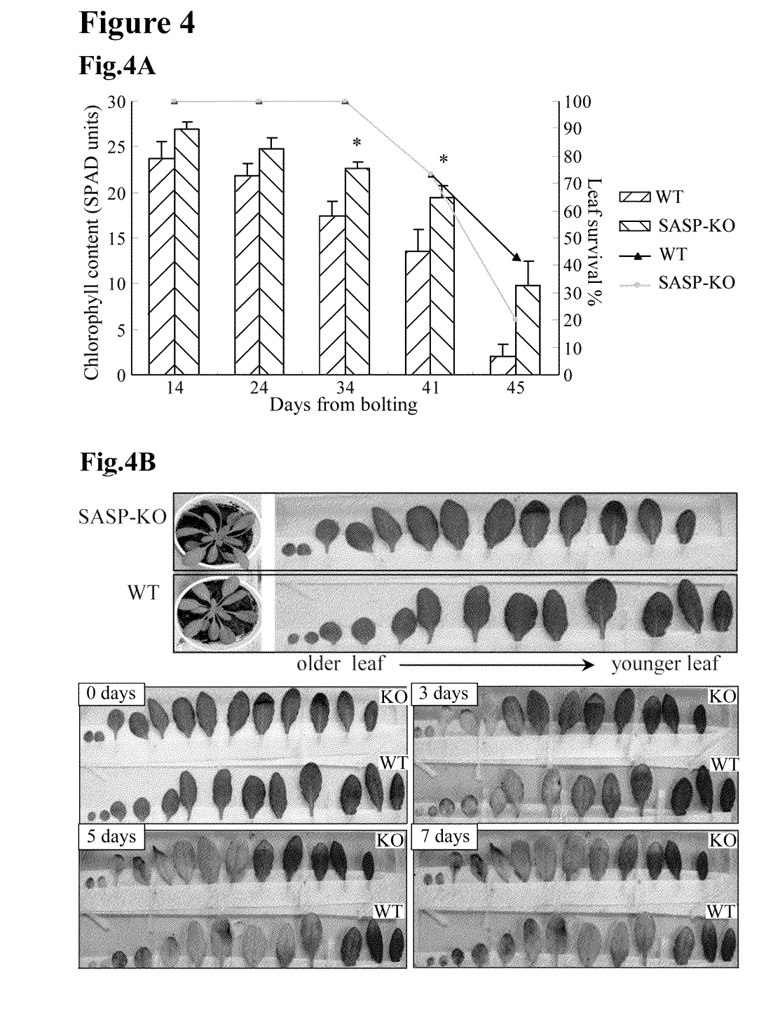

We used transgenic lines of Arabidopsis carrying a T-DNA insertion interrupting SASP expression (FIG. 3A). SASP proteolytic activity is not detected in plants homozygous for the T-DNA insertion (FIG. 3, B and C). Consistent with the predicted involvement of SASP in senescence, SASP-knock out plants (SASP-KO) show a delay in leaf senescence that becomes evident during the reproductive stage of plant development. Based on leaf chlorophyll content, leaf senescence starts at the same time in SASP-KO and wild type (WT) plants, but chlorophyll loss proceeds more slowly in SASP-KO leaves (FIG. 4A). Similar results were obtained in dark induced senescence treatments with detached leaves (FIG. 4B).

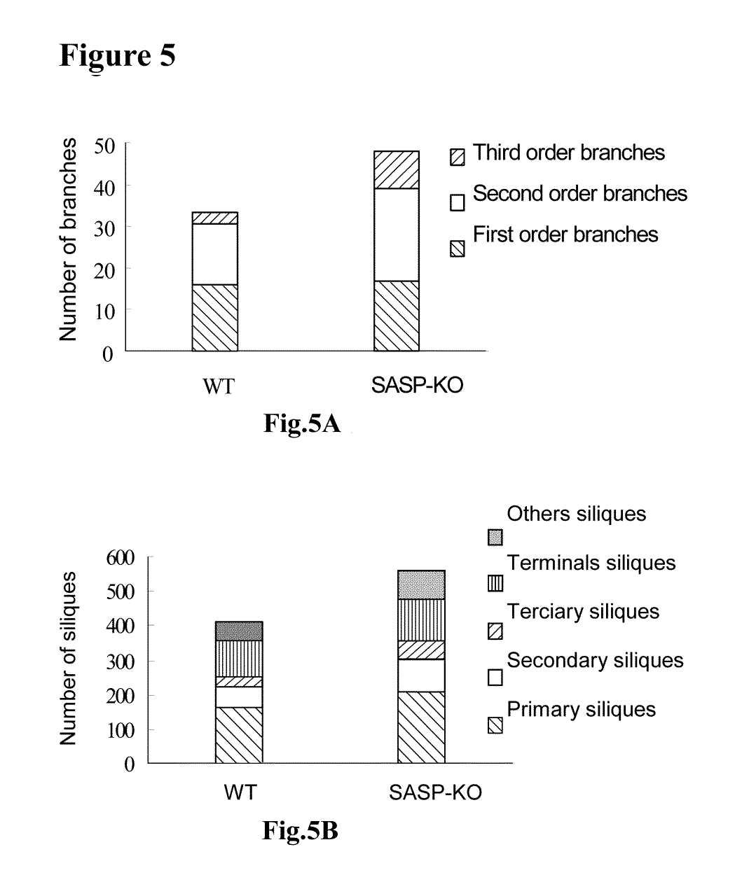

SASP-KO plants produce inflorescences with more branches than WT plants. The extra branches originate mostly from the axils of cauline leaves, but extra inflorescences also grow from the axils of rosette leaves, depending on the growth conditions. The more branched SASP-KO inflorescences produce more siliques, filled of seeds, which are the same size and weight as wild type seeds, and additionally have the same germination rate as wild type seeds. In plants grown under a photosynthetic photon flux density of 150 μmol m^{31 2}s⁻¹ and a long day photoperiod, by the end of the plant cycle (after growth was completely arrested) the total number of inflorescence (reproductive) branches developed by WT and SASP-KO plants were 33.4±4.7 and 48.2±10.3 respectively (p<0.05) (FIG. 5A). This difference is explained by a higher number of second and third order branches produced by SASP-KO plants. SASP-KO inflorescences also developed more branches of higher order (quaternary, quinary) than WT inflorescences, but as their contribution to silique yield is not significant they were not further considered in this study. The more branched inflorescences in SASP-KO produced more siliques than the WT inflorescences, 585.4±21.4 and 412.8±43.6 siliques per plant, respectively (p<0.05), (FIG. 5B).

Plant branching is influenced by environmental factors, e.g., plant density, light quality, nutrient availability (e.g. Casal 2004). Therefore, SASP-KO reproductive development was examined under different environmental situations. Table 1 summarizes the inflorescence development in terms of number of branches and siliques produced under different irradiance and photoperiod conditions. Considering irradiance as the main variable factor through the examined conditions (Table 1) SASP-KO plants developed more branched silique-bearing inflorescences than WT plants under high irradiance conditions, however SASP-KO plants consistently outperformed WT plants even under lower irradiance, and in both long and short photoperiods.

TABLE 1
Effect of light intensity and photoperiod on inflorescence branching and
silique production.

| | | | | | | | Increment |
|---|---|---|---|---|---|---|---|
| | | | | | | | (%) of |
| | | | Number of branches | | Number of siliques | | siliques in |
| | Photon | | per plant | | per plant | | SASP-KO |
| | Flux | | | SASP- | | SASP- | related to |
| Light source | density | Photoperiod | WT | KO | WT | KO | WT |
| Halogen | 150 μmols | SD (12 hs) for | 33.4 | 48.2 * | 412.8 | 585.4 * | 41% |
| light | m⁻² s⁻¹ | 2 weeks, then | | | | | |
| | | transferred LD | | | | | |
| Halogen | 150 μmols | LD | Not determined | | 250.0 | 502.0 | 100% |
| light | m⁻² s⁻¹ | | | | | | |
| Incandescent | 90 umols | LD | 40.5 | 56.4 * | 347.5 | 455.4 * | 31% |
| bulb | m⁻² s⁻¹ | | | | | | |
| Fluorescent | 90 μmols | LD | 41.2 | 52.2 | 424.0 | 479.0 | 13% |
| lamps | m⁻² s⁻¹ | | | | | | |

| | | | | | |
|---|---|---|---|---|---|
| Fluorescent | 50 μmols | LD | 110 | 186 * | Not determined |
| lamps | m⁻² s⁻¹ | | | | |

The experiments were done with 8-12 plants per genotype and light condition.
Asterisks represent statistically significant differences between genotypes (p < 0.05).
SD and LD: Short and Long Day, respectively.
In this experiment LD condition was applied with continuous light.

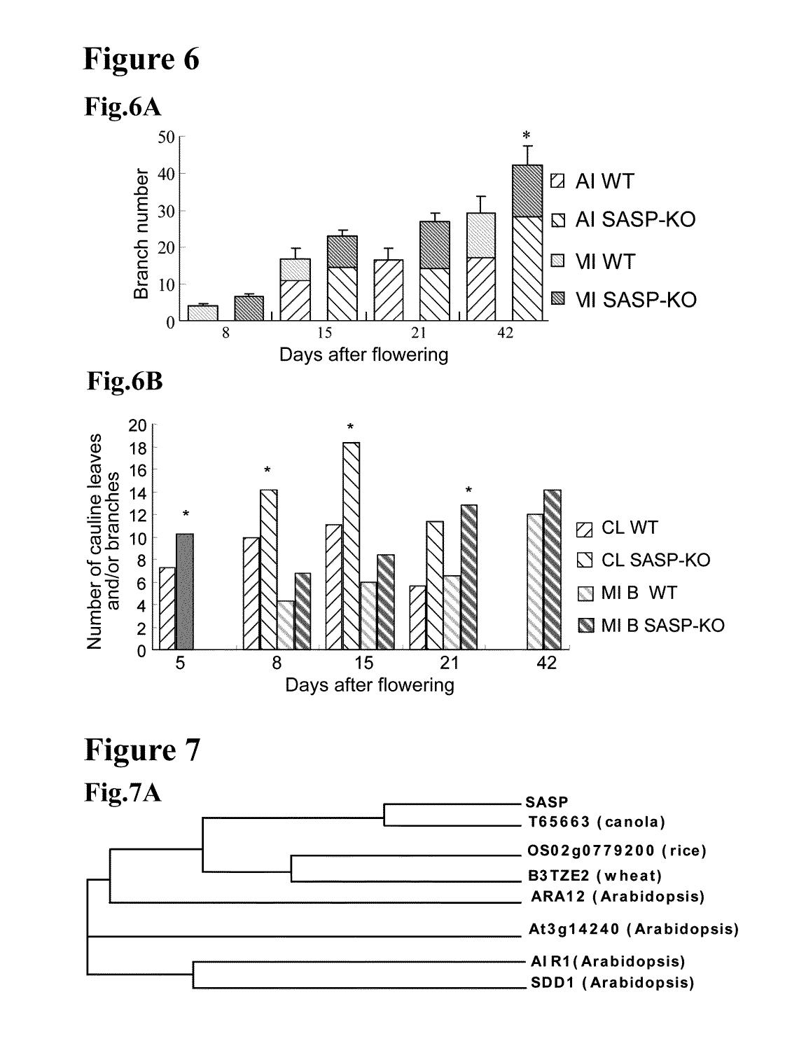

SASP-KO and WT plants have the same flowering time under long or short days (data not shown), therefore the different phenotypes appear after the inflorescence meristem is set up. Time course analysis of branch production shows differences between SASP-KO and WT early in the reproductive development, and this difference increases as inflorescence development proceeds, becoming more evident by the end of the plant life cycle (FIG. 6). In Arabidopsis plants growing under continuous light a variable number of inflorescences develop on the axils of rosette leaves (axillary inflorescences, AI), right after the main inflorescence (MI) elongates. AI development resembles MI development, following a modular pattern (Marshall, 1996). One week after flowering WT and SASP-KO MI developed 4.3±0.2 and 6.8±0.6 branches respectively, with no visual AI growth (FIG. 6A). Under long days, three weeks after flowering, the number of branches produced by the MI was 6.6±0.8 for WT and 12.6±2.4 for SASP-KO plants (p<0.05). AI developed 17.2±1.7 and 28.16±3.3 branches in WT and SASP-KO respectively. The faster growth of SASP-KO MI with respect to the WT is preceded by a faster growth of cauline leaves (FIG. 6B). WT plants developed an average of 10 cauline leaves per plant by the first week after flowering, and 11.1 cauline leaves a week thereafter. SASP-KO plants developed an average of 10 cauline leaves per plant already 5 days after flowering, and cauline leaves production continued up to an average of 18.4 leaves per plant. SASP-KO phenotype could be related to a delay in the timing of transition from inflorescence meristem to floral meristem, or to other regulatory process of induction/repression of meristem activation or bud growth, occurring at a certain point of development or continuously during the reproductive stages of development.

Plant branching is the final result of different developmental programs, i.e., specification of meristem identity and maintenance, bud outgrowth, regulation of inflorescence branching and floral organ identity. Most of these programs have been shown to be highly conserved across dicots and monocots. For example, meristem initiation and maintenance is controlled by the CLAVATA (CLV) pathway. The genes CLV1, CLV2, CLV3 and WUS operate this pathway in Arabidopsis, CLV genes inhibit WUS expression, which increases meristem size. An orthologous regulatory pathway has been described in rice and maize. TD1 and FEA2, and FON1 and FON2 are orthologues of CLV genes in maize and rice respectively, and mutant plants for these genes phenotypically resemble CLV mutants (Bomment et al., 2005, Suzaki et al., 2006). Hormonal regulation of axillary meristem initiation and outgrowth is controlled by a pathway which involves a long distance mobile signal, synthesized and processed by the MAX (More Axillary Meristem) genes, MAX1, MAX2, MAX3 and MAX4 in Arabidopsis (Bennet et al., 2006). Homologues of MAX genes have been described in rice and maize (Arite et al., 2007). Furthermore, mutant complementation analysis confirmed their function. For example, HIGH-TILLERING DWARF1 (HTD1) is the MAX3 homolog in rice and it rescued the phenotype of Arabidopsis MAX3 KO mutant plants (Zou et al., 2006). The transition from inflorescence meristem to floral meristem strongly influences inflorescence architecture. This transition depends on the antagonistic function of two genes, LEAFY (LFY) and TERMINAL FLOWER (TF) in Arabidopsis: the first promotes floral identity whereas the second promotes indeterminate growth. Over-expression of TF1 increases inflorescence branching in Arabidopsis. Again, this system is conserved in other, unrelated, plant species, such as rice, as was demonstrated when similar results were observed when rice TF1 homologues, RNC1 and RCN2, were expressed in Arabidopsis (Nakagawa et al., 2002). Likewise, the Teosinte Branch (TB1) gene encodes a transcription factor controlling bud outgrowth in maize; changes in TB1 were implicated in maize domestication. TB 1 also controls tillering in rice (OsTB1) (Takeda et al., 2003). TBL1/BRC1 gene plays a similar role in Arabidopsis, and loss-of-function mutants develop more branches from rosette leaves.

Plant Material

T-DNA insertion lines of Arabidopsis (Col ecotype) were obtained from the Salk Collection (www.salk.edu). The phenotypic analysis of SASP Knock out (“SASP-KO”) plants was mostly performed on the SALK_{—}147962.44.25x line. The selection for SASP Knock out plants included two screenings, one for the presence of the T-DNA insertion and the other one for proteolytic activity, performed in zymograms as described before for SASP detection.

For T-DNA screenings by PCR amplification, DNA was prepared from leaves as in Dellaporta et al. (1983). The primers used correspond to those suggested by SALK (www.salk.edu). Lb1 (Left border 1) starts at the right side of the TDNA insertion, 5′ GCGTGGACCGCTTGCTGCAACT (SEQ ID No. 16). The right primer, RP 5′ TCGGATTTTCTGCATTCAC (SEQ ID No. 17) and left primer (LP) 5′ TTCTTAAACCGGACGTGATTG (SEQ ID No. 18) were used to amplify wild type SASP from genomic DNA. The TDNA insertion is amplified with the Lb1-RP combination giving a 500-750 by product. LP-RP combination amplifies a 1Kb fragment of the WT wild type allele.

Plant Growth Conditions

Plants were grown in soil in 250 ml pots, one plant per pot, under 150 μmol m⁻²s⁻¹ PPFD (Photosynthetic Photon Flux Density) provided by tungsten-halogen lamps, or at other irradiances when described. Plants were grown under a 12 hours photoperiod for the first 2 weeks, and under continuous light thereafter, or under other photoperiods when described.

Dark induced senescence was performed with leaves taken from plants growing under short day, under 150 μmol m⁻²s⁻¹ PPFD.

Chlorophyll content was measured with the SPAD Portable Chlorophyll Meter (Minolta).

Seed morphology analysis was performed under a dissecting microscope. Average seed weight was calculated by weighing 100 seeds.

### 2. Bioinformatic Analysis

BLAST searching and multiple sequence alignment suggest potential orthologues of SASP in oil-seed rape (Brassica napus), rice and wheat (FIG. 7). The Arabidopsis subtilases SDD1, ARA12 and At3g14240 included in this alignment were selected within the 56 subtilases encoded in the Arabidopsis genome because they showed the highest similarity to SASP in terms of DNA sequence (BLAST analysis). The search for orthologues of SASP was done by BLAST searching at the National Center for Biotechnology Information (NCBI), and at The Genex Index Project, NSF (National Science Foundation) databases. Two other databases were also used to examine oilseed rape homologues: DFCI Plant Gene Index (http://compbio.dfci.harvard.edu/tgi/plant.html) and BRAD, Brassica database (http://brassicadb.org/brad/). For protein alignment, when amino acid sequences were not available in databases, DNA sequences (i.e., EST (Expressed Sequence Tag) and partial mRNA sequences) were translated in silico (www.expasy.ch).

Nucleic acid and amino acid sequence analysis show the same alignment pattern (FIG. 7A). At the nucleic acid level, the B. napus EST T65663 shares 92.2% identity with Arabidopsis SASP (97% coverage). The rice subtilase 0s02g0779200 (NCBI, Loc_Os02g53860 for The Rice Genomic Annotation Project, NSF) shares 58.4% identity with SASP, furthermore, Os02g53860 and SASP were clustered together in a cross-genome comparison and phylogenetic analysis of serine proteases in Arabidopsis and rice (Tripathi & Sowdhamini 2006). B3TZE7 is expressed in senescing leaves and thus, the partial mRNA B3TZE7 of wheat is up-regulated in a similar manner to Arabidopsis SASP during leaf aging (Arroz et al., 2008, http://www.uniprot.org/uniprot/B3TZE7). We have found that B3TZE7 shares 83.6% identity with AtSASP. The subtilases SASP, T65663, Os02g0779200 and B3TZE7 share conserved domains that are not present in the Arabidopsis subtilases SDD1, ARA12 and At3g14240 (FIGS. 7B and 7C, boxes and bold letters, in the amino acid and nucleic acid sequences, respectively).

Primers designed based on these conserved regions (FIG. 7C underlined letters) allowed the PCR amplification of partial sequences of SASP putative orthologues in oil-seed rape and rice, from genomic DNA (FIG. 7D).

Posttranslational modifications of SASP protein were predicted by using the TargetP and iPSORT programs (http://ca.expasy.org, http://hc.ims.u-tokyo.ac.up/iPSORT). Sequence similarity searches were done at National Center of Biotechnology Information (NCBI) and The Genex Index Project databases (ww.ncbi.nlm.nih.gov, http://compbio.dfci.harvard.edu/tgi/cgi-bin/tgi/Blast/index.cgi). Multiple sequence alignments were performed with ClustalW software (EMBL-EBI, http://www.ebi.ac.uk/). In silico translation was done with the Expasy Translation Tool programme (http://ca.expasy.org).

Statistic analysis: means were compared with Student (p<0.05 and p<0.01) or LSD test (p<0.05).

### 3. Identification and Analysis of SASP Orthologous Genes in Rice

BLAST searching and multiple sequence alignment of nucleic and amino acid sequences (described below) revealed potential orthologues of SASP in oil-seed rape (Brassica napus), and rice (FIG. 7) as described above.

The Locus Os02g0779200 (TIGR Loc_{—Os}02g53860) was identified as a candidate SASP orthologue in rice. Furthermore, according to DNA microarrays analysis with eFP Browser database and bioinformatic tool (Winter et al., 2007), Os02g0779200 expression is up-regulated in mature leaves respect to young leaves (see FIG. 8). This expression profile resembles SASP expression in Arabidopsis.

In short, the gene Os02g0779200 was amplified by PCR from genomic DNA of Oryza swim ssp. japonica cv. Nipponbare. The primers designed for the PCR were 5′ CCCCGGTGCGCCATGGCTACCCTC-3′ (SEQ ID No. 19) and 5′CTACATGGATGCTGCTCGGCCGTTC-3′ (SEQ ID No. 20), forward and reverse primers respectively. The sequence for the restriction enzyme XbaI was included in both primers. The resulting amplicon corresponds to the gene Os02g0779200 (mono-cistronic) from 12 nucleic acids upstream the first codon of the coding region up to the stop codon.

Amplification of Rice SASP

DNA

Seeds of the japonica rice variety Nipponbare were used. DNA was extracted directly from the crushed seeds using the Machery Nagel Nucleospin (Food) DNA extraction kit. The DNA concentration was measured using fluorometry (43 ng/μL), and was diluted to 5 g/μL with water.

Primers

The following primers were designed using Applied Biosystems Primer Express and have an anneal temperature of around 67° C. or higher

The primers are relatively long, and as such are preferred for amplification with proof reading polymerases. The nine pairwise combinations of primers (F1R1, F1R2 etc.) were made and held as 10× stocks (2 μM each primer)

PCR

The reactions were carried out using Novagen (CalBiochem, Merck) KOD hot start DNA polymerase. The reactions were carried out in a volume of 20 μL, in 384 well Sarstedt PCR plates in ABI9700 Viper blocks. The reaction plates were sealed with PCR foil seals Thermo adhesive foil AB-0626). Two formulations of PCR mix were successful, containing either dimethyl sulfoxide (DMSO) or Q reagent (supplied with the Qiagen microsatellite Type-It kit). The amounts for one reaction were:

| | 20 μL | | 20 μL |
|---|---|---|---|
| Reagent | reaction | Reagent | reaction |
| water | 5.4 | water | 6.4 |
| 10 x buffer | 2 | 10 x buffer | 2 |
| dNTPs | 2 | dNTPs | 2 |
| 25 mM MgSO4 | 1.2 | 25 mM MgSO4 | 1.2 |
| 10x primers | 2 | 10 x primers | 2 |
| DNA (5 ng/μL) | 5 | DNA (5 ng/μL) | 5 |
| +Q | 2 | +DMSO | 1 |
| enzyme(1 unit/μL) | 0.4 | enzyme(1 unit/μL) | 0.4 |

Two sets of PCR conditions were successful: a straightforward three step PCR and a Touchdown PCR procedure. Three step PCR:

| | | | |
|---|---|---|---|
| Denaturation | 95° C. | 2 min | |
| 40 cycles | 95° C. | 20 s | |
| | 61° C. | 15 s | |
| | 70° C. | 60 s | |
| Touchdown PCR: | | | |
| Denaturation | 95° C. | 2 min | |
| 10 cycles | 95° C. | 20 s | |
| | 71° C. | 15 s | touchdown 1° C. per cycle |
| | 70° C. | 60 s | |
| 30 cycles | 95° C. | 20 s | |
| | 71° C. | 15 s | |
| | 70° C. | 60 s | |

The sequence of the gene is known to be relatively GC rich, with a high Tm and Ta. Such amplicons often interfere with the normal dynamics of the PCR (in this case the Tm of the amplicon is expected to be around 84C, and the anneal temperature is 61° C.). In such cases, the addition of 5% DMSO or 10% Q reagent is usually advantageous.

All four of the PCR conditions tested (+Q or +DMSO used either with Touchdown or Straight PCR) yield adequate products with all nine primer combinations. The Straight PCR amplicons are fractionally more intense than the Touchdown amplicons, and the DMSO-containing reactions are a little more uniform than the Q reagent reactions. The F2R2 combination gives the brightest amplicon, followed by F1R2.

The amplicon is XbaI digested and cloned into a binary vector containing the Cauliflower Mosaic Virus promoter (CaMV 35S) and the nopaline synthase terminator (tNOS) (“35S: Os02g0779200.NOS”). The vector 35S:Os02g0779200.NOS is then introduced into Agrobacterium.

Arabidopsis mutant plants in which SASP is knocked out (“SASP.KO”) are transformed with Agrobacterium containing the vector “35S:Os02g0779200.NOS” or “35S:SASP.NOS” (as control). One conventional method for stable transformation of Arabidopsis is the “Floral Dip” method (Clough and Bent, 1998). The resulting plant phenotypes are examined to confirm that Os02g0779200 rescues the SASP.KO phenotype and is an orthologue of Arabidopsis SASP.

Plant phenotype analysis comprises examining the time course of leaf senescence and the silique production (plant yield) as described herein.

To generate rice mutant plants for Os02g0779200, iRNA methods can be used. Transgenic rice plants (i.e., stable lines mutant for Os02g0779200) can be generated by conventional methods known to the skilled person. For example, particle gun bombardment (Christou et al. 1991) and Agrobacterium-mediated transformation are efficient for a wide range of japonica and indica elite cultivars. Embryos from immature or mature rice seeds are generally used for embryogenic callus production. The embryos are aseptically removed and plated onto callogenesis medium containing auxins (such as 2,4-D) for 2-3 weeks in the dark. Embryogenic calli are used as a target for transformation. Transformed cells and tissue can be selected on Hygromycin, kanamycin or PPT using the hpt, npt or selectable marker genes, respectively. Plants are regenerated after two rounds of callus selection (2× 2-3 weeks) by growing calli exhibiting differential growth onto a culture medium without auxins and under light conditions.

### 4. Identification and Analysis of SASP Orthologous Genes in Oil-Seed Rape

Oil-seed rape belongs to the Brassicaceae family, as does Arabidopsis. The EST T65663 from the tetraploid species Brassica napus shows 92% similarity to AtSASP. The wild type relative Brassica rapa, a diploid specie, presents two loci (Bra027376 and Bra021529) with 86% and 84% similarity to AtSASP, respectively. Bra021529 is 95% similar to T65663.

Primers specific for Bra027376 and Bra021529 were designed to amplify these genes starting from genomic DNA (FIGS. 9A and B). The expression of the candidate homologous Bra027376 and Bra021529 was analyzed by RT qPCR with these pairs of primers. In relatively young plants (45 days after emergence) expression of Bra027376 and Bra021529 is higher in senescing leaves (S1 and S2) compared to a young leaf (Y) (FIG. 9C). Remarkably, there is a dramatic increase in Bra027376 and Bra021529 mRNA levels as plants age, even for leaves at similar stages of senescence (compare samples 3, 4 and 5). Thus, expression of Bra027376 and Bra021529 increases during leaf senescence, and this increase is exacerbated in older plants during reproductive growth.

These genes (Bra027376 and Bra021529), with the highest similarity to AtSASP in terms of nucleic acid sequence and temporal (i.e., up-regulation during leaf senescence) expression are selected for further cloning. Cloning the B. rapa orthologues of SASP and rescuing Arabidopsis SASP:KO plants with these genes is achieved by following the same protocol as the one described for rice. Transgenic Brassica in which the function of the orthologue/s of SASP is knocked out or knocked down, for example by RNAi, can be generated by methods known in the art. A transformation method is described below.

Time Course Analysis of Oil Seed Rape SASP Genes Expression

The expression of the two SASP orthologs identified in oil seed rape was examined by Real Time qPCR. The primers were designed selecting nucleic acid sequences that are specific for each gene (Bra027376 or for Bra021529) and that yield PCR products of similar size. The primers were first tested in PCR reactions using genomic DNA from seedlings of Brassica rapa.

Amplification of DNA Sequences Specific for Bra027376 and Bra021529

DNA

Genomic DNA from seedlings of Brassica rapa was used for PCR. The DNA was extracted with the CTAB method (Rogers et al., 1989). DNA was quantitated spectrophotometrically and diluted to 5 μg/μl with water.

Primers

The primers were 5′-ATGGCTAAGCTCTCT3-'3 (SEQ ID No. 21) and 5′-CGCTGCTTTCACCGTC-3′ (SEQ ID No. 22) for Bra027376 and 5′-ATGGCCGCGAAGCTC-'3 (SEQ ID No. 23), and 5′-CCTCTGCGTGCTTTGAT-3′ (SEQ ID No. 24) for Bra021529.

PCR

The reactions were carried out using Fermentas Dream Taq Polymerase (Fermentas). The reactions were carried out in a volume of 0.5 μl in single PCR tubes. The PCR conditions were the same for the two reactions (amplification of Bra027376 and Bra021529). The condition for the PCR reactions was a straightforward three step PCR.

| | | |
|---|---|---|
| Denaturation | 95 ° C. | 1 min |
| 35 cycles | 95 ° C. | 30 s |
| | 59 ° C. | 30 s |
| | 72 ° C. | 1.45 min |
| Final extension | 72 ° C. | 5 min |

RT qPCR Expression Analysis

Plant Material

Five samples corresponding to young (Y), early senescing (51) and late senescing (S2) leaves from plants of three ages were sampled. Chlorophyll content was considered the parameter of leaf senescence and was measured no destructively with the SPAD 502 meter (Minolta). Sampling is summarized in Table 2:

TABLE 2

| | Leaf Developmental | Plant age (Days after | Chlorophyll content |
|---|---|---|---|
| Sample | Stage (DS) | Emergency, DAE) | (SPAD units) |
| 1 | Y | 45 | 37.7 ^{a}* |
| 2 | S₁ | 45 | 28.8 ^{b}* |
| 3 | S₂ | 45 | 13.2 ^{c}* |
| 4 | S₂ | 65 | 14.1 |
| 5 | S₂ | 80 | 13.0 |

* = Different letters indicate statistically significant differences (p < 0.05)

RNA Extraction

RNA extraction was performed with the RNasy extraction kit (Qiagen), according to the manufacturer's instructions.

RT qPCR.

For reverse transcription the enzyme Reverse Transcriptase Superscript III (Invitrogen) and random primers (Invitrogen) were used. The RNAse inhibitor RNAse Out (Invitrogen) was added to the mixture. The RT-PCR reactions were run with a Bio-rad/iQ5 real-time PCR detection system. The amplicons were detected with SybrGreen (Invitrogen). The conditions for Bra027376 and Bra021529 detection were:

| Cycle 1: (1X) | | |
|---|---|---|
| Step 1: | 95.0° C. | for 10:00. |
| Cycle 2: (50X) | | |
| Step 1: | 95.0° C. | for 00:15. |
| Step 2: | 60.0° C. | for 00:15. |
| Step 3: | 72.0° C. | for 00:30. |
| Cycle 3: (81X) | | |
| Step 1: | 55.0° C.-95.0° C. | for 00:30. |
| Increase set point temperature after cycle 2 by 0.5° C. | | |
| Cycle 4: (1X) | | |
| Step 1: | 4.0° C. | for Hold. |

The Polyubiquitin gen (UBQ10) was used as a housekeeping gene. The conditions for the RT-PCR of this gene were:

| Cycle 1: (1X) | | |
|---|---|---|
| Step 1: | 95.0° C. | for 10:00. |
| Cycle 2: (50X) | | |
| Step 1: | 95.0° C. | for 00:15. |
| Step 2: | 55.0° C. | for 00:15. |
| Step 3: | 72.0° C. | for 00:30. |
| Cycle 3: (81X) | | |
| Step 1: | 55.0° C.-95.0° C. | for 00:30. |
| Increase set point temperature | | |
| after cycle 2 by 0.5° C. | | |
| Cycle 4: (1X) | | |
| Step 1: | 4.0° C. | for Hold. |

Plant Material

A genetically uniform doubled haploid Brassica oleracea genotype, DH 1012 (Sparrow et al., 2004) can be used. This genotype is derived from a cross between a rapid cycling B. oleracea alboglabra (A12) and a B. oleracea italica Green Duke (GD33). Transformations are carried out using the Agrobacterium tumefaciens strain LBA4404 (Hoekema et al. 1983) harbouring the plasmid pFVT1 containing the neomycin phosphotransferase gene (nptII) as the selectable marker and the nucleic acid sequence of interest.

A. tumefaciens LBA 4404-containing pFVT1 is streaked onto solid LB medium (Sambrook and Russell, 2001) containing appropriate selection and incubated at 28° C. for 48 hours. A single colony is transferred to 10 ml of Minimal A liquid medium (Ausubel et al. 1998) containing the appropriate selection and transferred to a 28° C. shaker for 48 hours. A 50 μl aliquot of the resulting bacterial suspension is transferred to 10 ml of Minimal A liquid medium containing no selection and grown over night in a 28° C. shaker. Overnight suspensions of O.D₆₅₀=0.1 is used for inoculations (dilutions made using Minimal A liquid medium).

Plant Transformation

Seeds are surface sterilised in 100% ethanol for 2 minutes, 15% sodium hypochlorite plus 0.1% Tween-20 for 15 minutes and rinsed three times for 10 minutes in sterile distilled water. Seeds are germinated on full strength MS (Murashige and Skoog, 1962) plant salt base, containing 3% sucrose and 0.8% phytagar (Difco) at pH 5.6. Prior to pouring, filter-sterilised vitamins were added to the medium; myo-Inositol (100 mg/l), Thiamine-HCL (10 mg/l), Pyridoxine (1 mg/l) and Nicotinic acid (1 mg/l). Seeds are sown at a density of 15 seed per 90 mm petri dish and transferred to a 10° C. cold room overnight before being transferred to a 23° C. culture room under 16 hour day length with 70 μmol m⁻² sec⁻¹ illumination.

Based on the transformation protocol developed for Brassica napus (Moloney et al. 1989), and further developed by BRACT (www.bract.org), cotyledonary petioles excised from 4-day-old seedlings are dipped into an overnight suspension of Agrobacterium. Explants are maintained, 10 explants per plate, on co-cultivation medium (germination medium supplemented with 2 mg/l 6-benzylaminopurine); with the petioles embedded and ensuring the cotyledonary lamella are clear of the medium. Cultures were maintained in growth rooms at 23° C. with 16 hour day length, under scattered light of 40 μmol m⁻² sec⁻¹ for 72 hours. After 72 hours explants are transferred to selection medium (co-cultivation medium supplemented with 500 mg/l carbenicillin (or appropriate Agrobacterium eliminating antibiotic) and 15 mg/l kanamycin as the selection agent. Controls are established on kanamycin-free medium, as explants that have and have not, been inoculated with Agrobacterium.

Shoot isolation and plant regeneration

Regenerating green shoots are excised and transferred to Gamborgs B5 medium, containing 1% sucrose, 0.8% Phytagar, 500 mg/l carbenicillin and 50 mg/l kanamycin. Where dense multiple shoots are isolated, further sub-culturing is made after shoot elongation to ensure a main stem was isolated thus reducing the likelihood of escapes and the frequency of multi-stemmed plants when transferred to the glasshouse. Shoots are maintained on Gamborgs B5 medium until roots developed. Plantlets are then transferred to sterile peat pots (Jiffy No. 7) to allow further root development, before being transferred to the glasshouse.

Plant Maintenance and Seed Production

Transgenic plants are maintained in a containment lit glasshouse (of 16-hour photoperiod, +18/12° C. day/night) and self-pollinated, to generate the T₁ seed required for use in this study. Plants are covered with clear, perforated ‘bread-bags’ (Cryovac (UK) Ltd) as soon as they came into flower to prevent cross-pollination. The background genotype DH1012 is a self compatible genotype and daily shaking of the ‘bread-bag’ was carried out to facilitate pollination. Pods are allowed to develop on the plant until fully swollen and are harvested when pods had dried and turned brown. Harvested pods are threshed when dry, and seed stored in the John Innes Centre seed store (+1.5° C., 7-10 relative humidity).

Molecular Analysis

Leaf tissue from putative transgenic shoots (in vitro) is used for initial DNA extractions to PCR test for presence of the transgenes. Plant DNA is extracted using the microLYSIS PLUS® kit from Microzone Limited; 3 mm² leaf tissue plus 20 μl MicroLYSIS-PLUS is placed in a thermal cycler for the following cycles: 65° C. for 15 mins, 96° C. for 2 mins, 65° C. for 4 mins, 96° C. for 1 min, 65° C. for 1 min, 96° C. for 30 s, 20° C. hold. 1 μl of the above product is then used in subsequent PCR reactions. PCR reactions are carried out in a reaction volume of 20 μl containing: 17 μl ABgene PCR Ready Mix®, 1 μl forward primer (nptII 5 mM stock) and 1 μl reverse primer (nptII 5 mM stock) and repeated for the gene of interest. Primers are supplied by Sigma Genosys, (nptII 5′ GAG GCT ATT CGG CTA TGA CTG G 3′ (SEQ ID No. 25) and 5′ ATC GGG AGC GGC GAT ACC GTA 3′ (SEQ ID No. 26). PCR for both nptII is carried out on a MJ Research PTC-200 PCR machine using the following programmes: for nptII: 94° C. for 5 minutes; 35 cycles of 94° C. for 30 seconds, 60° C. for 30 seconds and 72° C. for 1 minutes 30 seconds; with an auto extension finish of 72° C. for 10 minutes. PCR products are analysed by electrophoresis on a 1% agarose gel, containing ethidium bromide (0.5 μg/ml).

Southern Analysis

Plant DNA is extracted using the Qiagen DNeasy plant mini kit. Southern analysis is carried out using 20 μg of plant DNA digested with BstX1 restriction endonuclease (Roche). Electrophoresis of the digests is carried out on a 1% agarose gel at 1.5V/cm for approximately 20 hours. DNA is transferred to a Hybond N+ membrane following the manufacturer's instructions (Amersham). Southern hybridization, as described by Sharpe et al. 1995, is used to determine insertion number for nptII.

Copy Number Analysis by Multiplexed Real Time PCR

The copy number of the transgene is measured using multiplexed real time PCR (TaqMan) assays. The nptII target gene is detected using a Fam labelled, Tamra quenched probe, and simultaneously an internal positive control gene is detected using a Vic labelled, Tamra quenched probe. The reactions are carried out using 5-20 ng of genomic DNA from each sample, in a 20 μl reaction volume, with each sample assayed twice. The cycle threshold (Cts) for the Fam and Vic signals are found for each tube, and the average DeltaCt (CtFam-CtVIC) calculated for each sample. The samples are ranked by DeltaCt (where high delta Ct relates to samples with low numbers of copies, and low DeltaCt to high numbers of copies). Plant samples are classified with respect to reference samples (of known copy number).

### 5. Identification and Analysis of SASP Orthologous Genes in Maize

We have identified a AtSASP homologue in maize. This was identified by carrying out a search in the DFCI maize database (http://compbio.dfci.harvard.edu/).

We identified a EST, TC489899, that has 80% and 90% similarity to AtSASP and to the putative SASP in rice, respectively, with 70% and 90% hit coverage. This is shown in SEQ ID No. 9.

### REFERENCES

Arite T, Iwata H, Ohshima K, Maekawa M, Nakajima M, Kojima M, Sakakibara H, Ausubel F M, Brent R, Kingston R E, Moore D D, Seidman J G, Smith J A and Struhl K. (1998). Current protocols in molecular biology. John Wiley and Sons. Inc. publication.

Kyozuka J (2007) DWARF10, an RMS1/MAX4/DAD1 ortholog, controls lateral bud outgrowth in rice. Plant J 51: 1019-1029.

Arroz J A., Teixeira A., Ferreira R. B. (2008) Subtilisin protease expression during leaf age and nitrogen deprivation. EMBL/GenBank/DDBJ database: http://www.uniprot.org/uniprot/B3TZE7

Beers E P, Jones A M, Dickerman A W. (2003). The S8 serine, CIA cysteine, and A1 aspartic protease families in Arabidopsis. Phytochemistry: 65:43-48.

Bennt T, Siebere T, Willett B, Booker J, Luschnig Ch and Leyser O (2006) The Arabidopsis MAX Pathway Controls Shoot Branching by Regulating Auxin Transport. Curr. Biol. 16: 553-563.

Berger D. and Altmann T (2000). A subtilisin-like serine protease involved in the regulation of stomatal density and distribution in Arabidopsis thaliana. Genes & Development 14:1119-1131.

Bleecker A B and Patterson S E (1997) Last Exit: Senescence, Abscission, and MeristemArrest in Arabidopsis Plant Cell 9:1169-1179.

Bomment P, Lunde C, Nardman J, Vollbrecht E, Running M, Jackson D, Hake S, Werr W (2005). Thick tassel dwarf1 encodes a putative maize ortholog of the Arabidopsis CLAVATA1 leucine rich repeat receptor like kinase. Development 132: 1235-1245.

Carmichael et al, RNA silencing, methods and protocols, Methods in Molecular Biology, 2005

Casal J, Fankhauser Ch, Coupland G and Blazquez M. (2004). Signalling for developmental plasticity. Trends in Plant Sci. 9:1360-1385.

Christou, P., Ford, T., and Kofron, M. 1991. Production of transgenic rice (Oryza sativa L.) plants from agronomically important indica and japonica varieties via electric discharge particle acceleration of exogenous DNA into immature zygotic embryos. Bio/Technology 9: 957-962.

Dellaporta S L, Wood, and Hicks J B. 1983. A plant DNA minipreparation: version II. Plant Molecular Biology Reporter 114:19-21.

Diepenbrock W (2000) Yield analysis of winter oilseed rape (Brassica napus L.): a review. Field Crops Res. 67: 35-49.

Doust A D (2007) Grass architecture: genetic and environmental control of branching. Curr. Opin. Plant Biol. 10:21-25.

Henikoff et al, Plant Physiology, 2004, 630-636

Hoekema, A., P. Hirsch, P. Hooykaas, and R. Schilperoort. 1983. A binary plant vector strategy based on separate vir and T region of the Agrobacterium tumefaciens Ti-plasmid. Nature 303: 179-180.

Liu J X, Srivastava R, Che P and Howell H (2007). Salt stress responses in Arabidopsis utilize a signal transduction pathway related to endoplasmic reticulum stress signaling. Plant J. 51: 897-909.

Marshall C (1996) Sectoriality and physiological organization in herbaceous plants: an overview. Vegetation 127: 9-16.

Martinez D, Bartoli C, Vojislava G & Guiamét J J (2007). Vacuolar cysteine proteases of wheat (Triticum aestivum L) are common to leaf senescence induced by different factors. J. Exp. Bot. 58: 1099-1107.

Molnar A, Melnyk C and Baulcombe D C Genome Biology 2011, 12:215 (2011).

Moloney, M M., J. M. Walker, and K. K. Sharma. 1989. High-efficiency transformation of Brassica napus using Agrobacterium vectors. Plant Cell Reports 8:238-242.

Murashige, T., and F. Skoog. 1962. A revised medium for rapid growth and bioassays and tobacco tissue culture. Physiol Plant 15:437-497.

Nakagawa M, Shimamoto K, Kyozuka J (2002) Overexpression of RCN1 and RCN2, rice TERMINAL FLOWER1/CENTRODIALIS homologs, confers delay of phase transition and altered panicle orphology in rice. Plant J. 29: 743-750.

Nooden L D, Penney J P. (2001). Correlative controls of senescence and plant death in Arabidopsis thaliana (Brassicaceae). J. Exp. Bot. 364: 2151-2159.

Rogers S O, Rehner S, Bledsoe C, Mueller G J, Ammirati J F (1989). Extraction of DNA from basidiomycetes for ribosomal hybridizations. Can J Bot 679235-1243

Rose R, Schaller A, Ottmann C. (2010) Structural features of plant subtilases. Plant Signaling & Behavior 5:180-183.

Rothstein The Plant Cell, Vol. 19: 2695-2699, September 2007

Sambrook J, Fritsch E F and Maniatis T (1989) Molecular Cloning, A Laboratory Manual

Sharma R C (1995) Tiller mortality and its relationship to grain yield in spring wheat. Field Crops Res. 41: 55-60.

Sharpe A G, I A P Parkin, D J Keith and D J Lydiate. 1995. Frequent nonreciprocal translocations in the amphidiploid genome of oilseed rape (Brassica napus). Genome 38: 1112-1121

Sparrow P A C, Dale P J and Irwin J A (2004). The use of phenotypic markers to identify Brassica oleracea genotypes for routine high-throughput Agrobacterium-mediated transformation. Plant Cell Reports. 23:64-70

Suzaki T, Toriba T, Fujimoto M, Tsutsumi N, Kitano H, Hirano H Y (2006). Conservation and diversification of meristem maintenance mechanism in Oryza sativa: Function of the FLORAL ORGAN NUMBER2 GENE. Plant Cell Physiol. 47: 1591-1602.

Takeda T, Suwa Y, Suzuki M, Kitano H, Ueguchi-Tanaka M, Ashikari M, Matsuoka M, Ueguchi C (2003) The OsTB1 gene negatively regulates lateral branching in rice. Plant J 33: 513-520.

Tornero P, Conejero V, Vera P. (1996). Primary structure and expression of a pathogen-induced protease (PR-P69) in tomato plants: Similarity of functional domains tosubtilisin-like endoproteases (viroid/pathogenesis-related protein/defense). PNAS 93: 6332-6337.

Tripathi P L & Sowdhamini R. (2006). Cross genome comparisons of serine proteases in Arabidopsis and Rice. BMC Genomics doi:10.1186/1471-2164-7-200.

Watanabe M., Tanaka H, Watanabe D, Machida Ch. & Machida Y. (2004). The ACR4 receptor-like kinase is required for surface formation of epidermis-related tissues in Arabidopsis thaliana Plant J. 39: 298-308.

Winter D, Vinegar B, Nahal H, Ammar R, Wilson G V, (2007) An “Electronic Fluorescent Pictograph” Browser for Exploring and Analyzing Large-Scale Biological Data Sets. PLoS ONE 2(8): e718. doi:10.1371/.

Zhang P, Battchikova N, Jansen T, Appel J, Ogawat and Eva-Mari Aro (2004). Expression and Functional Roles of the Two Distinct NDH-1 Complexes and the Carbon Acquisition Complex NdhD3/NdhF3/CupA/5111735 in Synechocystis sp PCC 6803. Plant Cell 16:3326-3340.

Zimmermann P, Hirsch-Hoffmann M, Hennig L, and Gruissem W (2004). GENEVESTIGATOR. Arabidopsis Microarray Database and Analysis Toolbox. Plant Physiol. 136: 2621-2632.

Zou J, Zhang S, Zhang W., Li G, Chen Z, Zhai W, Zhao X, Pan X, Xie Q, and Zhu L (2006) The rice HIGH-TILLERING DWARF1 encoding an ortholog of Arabidopsis MAX3 is required for negative regulation of the outgrowth of axillary buds. Plant J. 48: 687-696.

The invention is further described by the following numbered paragraphs:

1. A transgenic plant cell, plant or a part thereof wherein the activity of a SASP polypeptide is inactivated, repressed or down-regulated.

2. A transgenic plant cell, plant or a part thereof according to paragraph 1 wherein the plant has increased yield.

3. A transgenic plant cell, plant or a part thereof according to paragraph 1 or 2 wherein the expression of a gene encoding a SASP polypeptide is inactivated, repressed or down-regulated.

4. A transgenic plant cell, plant or a part thereof according to paragraph 3 wherein the gene encoding a SASP polypeptide is from wheat, rice, brassica or zea mays.

5. A transgenic plant cell, plant or a part thereof according to paragraph 3 wherein the SASP gene encoding a SASP polypeptide comprises a nucleic acid sequence as shown in SEQ ID No. 1, a functional variant, homologue or orthologue thereof.

6. A transgenic plant cell, plant or a part thereof according to paragraph 5 wherein the functional variant, homologue or orthologue comprises a nucleic acid sequence as shown in SEQ ID Nos. 3, 4, 5, 7 or 9.

7. A transgenic plant cell, plant or a part thereof according to a preceding paragraph wherein the endogenous SASP gene carries a functional mutation.

8. A transgenic plant cell, a plant or a part thereof according to a preceding paragraph wherein expression of the endogenous SASP gene is silenced.

9. A transgenic plant cell, a plant or a part thereof according to a preceding paragraph derived from a crop plant.

10. A transgenic plant cell, a plant or a part thereof according to a preceding paragraph derived from a monocotyledonous plant.

11. A transgenic plant cell, a plant or a part thereof according to a preceding paragraph derived from a dicotyledonous plant.

12. A transgenic plant tissue, plant, harvested plant material or propagation material of a plant comprising the plant cell according to any of paragraphs 1 to 11.

13. A transgenic plant cell tissue, plant, harvested plant material or propagation material of a plant according to paragraph 12 wherein said plant is a brassica, wheat, rice or maize.

14. A method for making a transgenic plant with increased yield comprising inactivating, repressing or down-regulating the activity of a senescence associated subtilisin protease (SASP) polypeptide in a plant.

15. A method according to paragraph 14 wherein the method comprises inactivating, repressing or down-regulating the expression of a gene encoding a SASP polypeptide.

16. A method according to paragraph 15 wherein the gene encoding a SASP polypeptide is from wheat, rice, brassica or zea mays.

17. A method according to paragraph 14 or 15 wherein the SASP gene comprises a nucleic acid sequence as shown in SEQ ID No. 1, a functional variant, homologue or orthologue thereof.

18. A method according to paragraph 17 wherein the functional variant, homologue or orthologue comprises a nucleic acid sequence as shown in SEQ ID Nos. 3, 4, 5, 7 or 9.

19. A method according to any of paragraphs 14 to 18 wherein said method comprises introducing a functional mutation in a gene encoding a SASP protein or peptide in a plant.

20. A method according to paragraph 19 wherein said mutation is introduced using T-DNA insertion or chemical mutagenesis.

21. A method according to paragraph 20 comprising using TILLING.

22. A method according to any of paragraph 14 to 18 comprising silencing of the SASP gene.

23. A method according to paragraph 22 comprising introducing a RNAi, snRNA, dsRNA, siRNA, miRNA, ta-siRNA or cosuppression molecule which targets the SASP gene into a plant.

24. A method for increasing yield comprising making a plant with increased yield as described in any of paragraphs 14 to 23.

25. A plant obtained or obtainable by the method of any of paragraphs 14 to 23.

26. An isolated nucleic acid comprising a sequence as shown in SEQ ID No. 1, a functional variant, homologue or orthologue thereof.

27. An isolated nucleic acid comprising according to paragraph 26 wherein the functional variant, homologue or orthologue comprises SEQ ID No. 9.

28. An expression cassette comprising an isolated nucleic acid according to paragraph 26 or 27.

29. A plant cell, plant or a part thereof with increased yield wherein the activity of a SASP polypeptide is inactivated, repressed or down-regulated and wherein said plant has been generated by methods that do not solely rely on traditional breeding.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

SEQUENCE LISTING

SEQ ID No. 1 Arabidopsis SASP DNA nucleotide
sequence >gi|45773915|gb|BT012275.1| Arabidopsis
thaliana At3g14067 gene, complete coding region
ATGGCTAAGCTCTCTCTTTCCTCCATCTTCTTCGTCTTCCCTCTCCTCCT
CTGTTTCTTTTCCCCTTCTTCTTCTTCATCGGATGGCTTAGAATCCTACA
TCGTCCATGTGCAGAGATCTCATAAGCCTTCCCTCTTCTCCTCCCACAAC
AACTGGCACGTCTCTCTCCTTCGCTCTCTCCCTTCTTCTCCCCAACCAGC
AACGCTGCTCTACTCTTATTCACGCGCCGTTCATGGCTTCTCCGCTCGTC
TCTCCCCTATCCAAACCGCCGCCCTCCGCCGTCATCCTTCAGTCATCTCC
GTTATACCTGATCAAGCGCGTGAGATCCACACAACTCACACGCCTGCCTT
CCTCGGTTTCTCCCAAAACTCTGGACTCTGGAGCAACTCAAATTACGGGG
AAGACGTGATCGTCGGCGTTTTAGATACTGGAATCTGGCCGGAACATCCA
AGTTTCTCGGATTCAGGTCTCGGTCCAATTCCATCTACCTGGAAAGGCGA
GTGCGAGATCGGACCTGATTTTCCTGCCTCATCTTGCAATCGGAAGCTTA
TCGGAGCTCGAGCGTTTTACAGGGGATATTTAACGCAACGGAATGGAACA
AAAAAGCATGCAGCCAAGGAATCGAGATCGCCGCGTGATACAGAAGGTCA
TGGCACGCACACGGCATCTACGGCAGCTGGATCGGTGGTTGCTAACGCGA
GTTTGTACCAGTACGCGCGCGGAACAGCTACTGGGATGGCGTCAAAGGCG
AGAATCGCCGCTTACAAAATCTGTTGGACCGGCGGATGTTACGATTCCGA
TATCCTCGCCGCCATGGATCAGGCGGTTGCCGACGGTGTTCACGTTATCT
CTCTCTCCGTCGGAGCCAGCGGTTCCGCCCCGGAGTATCACACGGACTCT
ATAGCGATCGGAGCATTTGGAGCCACGCGGCACGGCATCGTCGTTTCTTG
CTCCGCTGGGAATTCTGGTCCTAATCCTGAAACCGCGACGAACATCGCTC
CATGGATCTTAACCGTTGGTGCGTCCACCGTCGATAGAGAATTCGCCGCA
AACGCAATCACGGGAGACGGGAAAGTCTTCACGGGAACATCACTGTACGC
AGGCGAATCTCTACCGGATTCTCAACTTTCTCTGGTATATTCCGGCGATT
GCGGAAGTAGATTGTGTTACCCTGGGAAATTGAATTCATCATTGGTTGAA
GGCAAAATCGTGCTCTGTGACAGAGGAGGCAACGCAAGAGTTGAGAAAGG
AAGTGCAGTCAAGCTAGCCGGTGGTGCTGGTATGATTCTGGCGAACACAG
CTGAAAGCGGTGAAGAATTAACCGCCGATTCGCATCTCGTCCCGGCGACA
ATGGTTGGAGCTAAAGCTGGAGATCAAATCCGCGACTACATCAAAACATC
AGACTCTCCCACTGCAAAAATCAGTTTCCTAGGCACTTTGATCGGACCAT
CTCCTCCTTCTCCCAGAGTCGCCGCTTTCTCCAGCCGTGGACCGAATCAC
TTGACACCGGTTATTCTTAAACCGGACGTGATTGCTCCTGGAGTCAACAT
TTTAGCCGGTTGGACCGGGATGGTTGGTCCTACCGATTTAGATATCGATC
CAAGACGGGTTCAATTCAACATCATCTCCGGTACATCGATGTCGTGCCCA
CACGTTAGTGGACTCGCCGCTCTCCTCCGTAAAGCTCATCCCGATTGGTC
ACCTGCAGCAATCAAATCCGCCCTTGTAACCACCGCTTACGATGTCGAAA
ACTCCGGCGAACCAATCGAGGATCTCGCCACCGGTAAATCATCGAACTCA
TTCATCCACGGAGCTGGACACGTCGATCCAAACAAAGCTTTGAATCCTGG
TTTGGTTTACGACATCGAGGTCAAAGAGTACGTAGCTTTCCTCTGCGCCG
TGGGATACGAGTTTCCGGGGATTCTAGTCTTTCTTCAAGATCCAACTCTT
TACGACGCATGTGAAACGAGCAAGCTAAGAACCGCCGGCGATCTCAATTA
CCCATCTTTCTCCGTGGTTTTCGCATCGACCGGGGAAGTTGTGAAATACA
AAAGGGTTGTCAAAAACGTGGGAAGCAATGTCGACGCTGTGTACGAAGTC
GGAGTTAAATCTCCGGCGAATGTTGAGATTGATGTTTCTCCAAGCAAGCT
TGCGTTCAGCAAGGAGAAGAGCGTGTTGGAGTATGAAGTCACATTTAAGA
GCGTTGTGCTCGGCGGAGGAGTCGGATCCGTGCCGGGTCATGAATTCGGG
TCGATCGAATGGACAGACGGTGAACACGTTGTTAAGAGTCCGGTGGCCGT
CCAATGGGGTCAGGGATCAGTTCAGTCCTTCTGA
SEQ ID No. 2 Arabidopsis SASP amino acid sequence
>SASP
MAKLSLSSIFFVFPLLLCFFSPSSSSSDGLESYIVHVQRSHKPSLFSSHN
NWHVSLLRSLPSSPQPATLLYSYSRAVHGFSARLSPIQTAALRRHPSVIS
VIPDQAREIHTTHTPAFLGFSQNSGLWSNSNYGEDVIVGVLDTGIWPEHP
SFSDSGLGPIPSTWKGECEIGPDFPASSCNRKLIGARAFYRGYLTQRNGT
KKHAAKESRSPRDTEGHGTHTASTAAGSVVANASLYQYARGTATGMASKA
RIAAYKICWTGGCYDSDILAAMDQAVADGVHVISLSVGASGSAPEYHTDS
IAIGAFGATRHGIVVSCSAGNSGPNPETATNIAPWILTVGASTVDREFAA
NAITGDGKVFTGTSLYAGESLPDSQLSLVYSGDCGSRLCYPGKLNSSLVE
GKIVLCDRGGNARVEKGSAKLAGGAGMILANTAESGEELTADSHLVPATM
VGAKAGDQIRDYIKTSDSPTAKISFLGTLIGPSPPSPRVAFSSRGPNHLT
PVILKPDVIAPGVNILAGWTGMVGPTDLDIDPRRVQFNIISGTSMSCPHV
SGLAALLRKHPDWSPAAIKSALVTTAYDVENSGEPIEDLATGKSSNSFIH
GAGHVDPNKALNPGLVYDIEVKEYVAFLCAVGYEFPGILVFLQDPTLYDA
CETSKLRTAGDLNYPSFSVVFASTGEVVKYKRVVKNVGSNVDAVYEVGVK
SPANVEIDVSPSKLAFSKEKSVLEYEVTFKSVVLGGGVGSVPGHEFGSIE
WTDGEHVVKSPVAVQWGQGSVQSF
SEQ ID No. 3 Brassica Bra027376 nucleotide
sequence (DNA)
ATGGCTAAGCTCTCTCTCTCCTCTGTCTTCTTCGTTTTCCCTCTCTTCCT
CTGTTTCTTCTCGTCGTTATCTTCTTGGGATGGGTTAGAATCATACATCG
TTCATGTGCAGAGTTCTCATAAGCCTTCTCTCTTCTCCTCCCACGACCAT
TGGCACAACTCTCTCCTCCGCTCTCTACCGTCCTCTCCACAACCGGCGAC
GCTCTTATACTCTTACTCACGCGCCGTTCAAGGCTTCTCCGCTCGTCTCT
CACCTACACAGACCGCCGCTCTTCGCCGTCACACTTCCGTTATCTCCGTT
ATACCAGATCAAGCGCGTGAGATTCACACCACTCATACACCTTCCTTCCT
CGGTTTCTCAGATAACTCCGGTCTCTGGAGCAACTCCAATTACGGCGAGG
ACGTGATCGTCGGCGTTCTCGACACCGGAATCTGGCCGGAGCATCCTAGC
TTCTCCGATTCAGGTCTCGATCCAGTTCCATCTACGTGGAAAGGCGCGTG
CGAGATCGGACCTGACTTTCCCGCTTCGTCTTGCAACCGGAAGCTCATCG
GAGCTCGAGCGTTCTATAAAGGATACTTAACGCATCGCAATGGGACGGTG
AAAGCAGCGAAGGAATCGCGATCGCCGCGGGATACGGAAGGTCATGGCAC
GCACACGGCATCCACTGCGGCAGGATCGGTGGTGGCGAACGCGAGCTTGT
ACCAATACGCGCGAGGAGTGGCGCGTGGGATGGCGTCGAAGGCGAGAATC
GCAGCTTATAAAATCTGCTGGACAGGTGGTTGTTACGATTCCGATATCCT
CGCGGCCATGGATCAGGCCGTTGCTGACGGTGTTCACGTGATCTCTCTTT
CCGTTGGTGCTAACGGTTACGCTCCCGAGTATCATATGGACTCAATCGCG
ATTGGAGCGTTTGGAGCCACGCGCCACGGTATCGTTGTTTCCTGCTCCGC
TGGAAACTCTGGTCCTGGTCCTCAAACCGCAACTAACATCGCTCCTTGGA
TCTTAACCGTCGGTGCGTCCACGATCGATCGAGAGTTCTCCGCGAACGCA
ATCACCGGCAACGGGAAAGTCTTCACCGGAACGTCGCTCTACGCCGGCGA
GCCTCTCCCTGATTCTCAGCTTTCTCTGGTGTATTCCGGCGATTGCGGAA
GCAGATTGTGCTACCCAGGGAAGCTGAACGCGTCCTTGGTGGAAGGGAAG
ATCGTTCTCTGTGACAGAGGAGGTAACGCCAGAGTTGAGAAAGGAAGCGC
CGTCAAGATCGCCGGCGGAGCAGGGATGATTCTCGCGAACACAGCTGAAA
GCGGGGAAGAGCTCACCGCCGATTCGCATCTCGTCCCGGCGACGATGGTC
GGAGCTAAAGCTGGAGATCAAATCCGCGAGTACATCCAAAAGTCAGACTC
TCCCACCGCAACAATCAGCTTCTTGGGCACTTTGATCGGACCTTCTCCTC
CTTCTCCCAGAGTCGCGGCCTTCTCAAGCCGTGGACCGAATCATATAACT
CCGGTTATCCTTAAACCGGACGTGATTGCGCCAGGAGTTAATATATTAGC
CGGTTGGACCGGAATGGTTGGTCCAACCGATTTGGATATCGATCCGAGAC
GGGTTCAATTCAATATAATCTCCGGTACATCGATGTCGTGCCCACACGTG
AGCGGACTCGCCGCTCTCCTCCGTAAAGCTCATCCCGATTGGTCACCGGC
GGCGATCAAATCCGCGCTCGTTACAACCGCTTACGATACAGAAAACTCCG
GCGAACCAATCGAGGATCTCGCCACCGGTAAGTCGTCGAACTCGTTCATC
CACGGAGCTGGACACGTGGATCCGAACAAAGCCTTGAACCCTGGGTTGGT
TTACGACATCGACGTCAAAGACTACGTGGCCTTCCTCTGCGCCGTGGGAT
ACGAGTTCCCGGGGATTCTAGTGTTCCTTCAAGATCCAACTCTTTACAAC
GCCTGCGAGACGAGCAAGCTAAGAACCGCCGGCGATCTCAATTACCCGTC
GTTCTCCGTCGTTTTCGGATCGAGCGTCGATGTTGTGAAGTACAGAAGGG
TTGTTAAGAACGTTGGGACCAACGTTGAGGCGGTGTACGAAGTCGGGGTT
AAGTCTCCGGCGAACGTGGAGATCGATGTGTCTCCGAGGAGGCTTGCGTT
TAGCAAGGGGGAGAGCGAGTTGGAATACGAAGTGACGTTTCGGAGCGTTG
TGCTTGGCGGAGGAGTTGGATCCGTACCGGGTCATGAATTCGGGTCGATC
GAGTGGACAGACGGTGAGCACGTCGTCAAGAGCCCGGTGGCTGTTCAGTG
GGGTCAGGGATCATCAGTTCAGTCATTCTGA
SEQ ID No. 4 Brassica Bra021529 nucleotide
sequence (DNA)
ATGGCCGCGAAGCTCTCTCTCTCCTCCGTCTTAATCGTTTTCTCTCTCTT
CCTCTGTTTCTCATCGTCATCATCTTCCTGGGATGGCTTAGAGTCATACA
TCGTCCATGTGCAAGGATCTCACAAGCCTTCTCTCTTCTCCTCCCACAGC
CACTGGCACAACTCTCTCCTCCGCTCCCTCCCATCCTCTCCCCAACCCGC
GACTCTCCTCTACTCCTACTCACGCGCCGTCAACGGCTTCTCCGCGCGTC
TCTCACCTTCCCAGACCTCCGCTCTCCGTCGCCACCCTTCCGTCATCTCC
CTAATACCAGATCAGGCGCGTGAGATCCACACCACTCACACCCCCGCCTT
CCTCGGCTTCTCCGATAACTCCGGTCTCTGGAGCAACTCCAATTACGGCG
AAGACGTGATCGTCGGCGTTCTCGATACCGGAATCTGGCCGGAGCATCCT
AGCTTCTCCGATTCAGGTCTCGATCCCGTTCCTTCCACATGGAAAGGCGC
GTGCGAGATCGGACCTGACTTCCCGGCGTCCTCCTGCAACCGGAAGCTCA
TCGGAGCTCGAGCGTTCTACAAGGGATACCTAACGCACCGCAACGGATCA
AAGCACGCAGAGGAATCCAAATCGCCGAGGGATACAGCAGGTCACGGGAC
GCACACCGCGTCAACCGCGGCTGGATCCGTTGTGGTCAACGCGAGTTTGT
ACCAATACGCGCGTGGCGTGGCGCGTGGGGTGGCGTCGAAGGCGAGAATC
GCTGCCTACAAAATCTGTTGGACTGGAGGTTGTTACGATTCGGATATCCT
CGCGGCTATGGATCAGGCCGTTGCGGATGGTGTCCACGTCATCTCTCTTT
CCGTTGGCGCTAACGGCTTCGCTCCGGAGTATCATAAAGACTCTATCGCG
ATCGGAGCGTTTGGAGCGATGCGTCACGGCATCGTCGTTTCTTGCTCCGC
CGGAAACTCAGGTCCGGGACCGCAAACGGCCACTAATATCGCTCCGTGGA
TCCTAACCGTCGGTGCGTCGACGGTGGATAGAGAGTTCACCGCGAACGCG
ATCACCGGAGACGGGAAAGTCTTCACCGGAACGTCGCTGTACGCAGGAGA
GCCTCTCCCTGATTCTCAGATTCCTCTGGTGTACTCCGGCGATTGCGGAA
GCAGATTGTGCTACCCCGGGAAGCTGAACTCGTCGTTGGTGGAAGGGAAG
ATCGTTCTCTGTGATAGAGGAGGAAACGCAAGAGTCGAGAAAGGAAGCGC
CGTCAAGATCGGCGGCGGAGCAGGGATGATTCTCGCGAACACAGCTGAAA
GCGGCGAAGAACTCACCGCCGATTCGCATCTCGTCCCGGCGACGATGGTC
GGAGCTAAAGCCGGAGATCAAATCCGCGACTACATCAAAAACTCAAACTC
TCCAACCGCAACGATCAGCTTCTTGGGAACTTTGATCGGCCCATCTCCTC
CTTCTCCAAGAGTCGCAGCCTTCTCTAGCCGTGGACCAAATCACATAACC
CCGGTTATCCTCAAACCGGACGTGATTGCGCCAGGTGTTAATATATTAGC
CGGTTGGACCGGAATGGTTGGTCCAACCGATTTAGATATCGACCCGAGAC
GAGTCAAATTCAACATCATCTCCGGTACATCGATGTCGTGCCCGCACGTG
AGCGGACTCGCCGCTCTCCTCCGTAAAGCTCACCCCGATTGGTCCCCGGC
GGCGATCAAATCCGCGCTCGTGACAACCGCTTACGACACCGAAAACTCCG
GGGAACCGATCGAGGATCTCGCCACCGGTGAATCGTCGAACTCGTTCATC
CACGGAGCGGGACACGTGGATCCGAACAAAGCGTTGAATCCCGGTTTGGT
TTACGACCTCGACGCTAAAGAGTACGTCGCGTTCCTCTGCGCCGTGGGGT
ACGAGTTCCCGGGGATTCTGGTGTTCCTTCAAGATCCGAGTCTTTACGAC
GCTTGTGAGACGAGCAAGCTTAGAACCGCCGGGGATCTCAATTACCCGTC
TTTCTCCGTCGTTTTCGGATCGAGTGTTGATGTTGTTAAGTACAGGAGAG
TTGTTAAGAACGTGGGGAGCAATGTTGACGCGGTGTATCAAGTCGGAGTT
AAGGCTCCGGCGAATGTGGAGATCGATGTGTCTCCGAGCAAGCTTGCGTT
TAGTAAAGAGACTAGGGAGATGGAGTACGAAGTGACGTTTAAGAGCGTTG
TGCTTGGAGGTGGAGTTGGATCCGTTCCGGGTCATGAGTTCGGGTCGATT
GAGTGGACAGACGGTGAACATGTCGTCAAGAGTCCCGTGGCTGTTCAATG
GAGTCAGGGGTCAGTTCAGTCATTCTGA
SEQ ID No. 5 Rice Os02g0779200 nucleotide
sequence (DNA) NCBI Reference Sequence:
NC_008395.2 >gi|115449042|ref|NM_001054836.1|
Oryza sativa Japonica Group Os02g0779200
(Os02g0779200) mRNA, complete cds (coding
sequence in bold)
CTTATTTAGTTCTCCAGGCCGCATTGGCGTCGAGTCATCGACCAATCCAA
TCCGCTCCCCCGGTGCGCCATGGCTACCCTCCGCCATCTCGCCGCCGTGC
TCCTCATCCTCTTCGCCGCCGCGTCGCCGGCGGCGGCGGCCGCGAGAGAG
CAGTCGACGTACATCCTCCACCTCGCGCCCGAGCACCCGGCGCTCAGGGC
CACGCGCGTCGGCGGCGGCGGCGGCGCCGTGTTCCTCGGCCGCCTCCTTC
GCCTCCCGCGCCATCTGCGCGCACCGCGGCCACGGTTGCTCTACTCCTAC
GCGCACGCAGCGACGGGGGTCGCGGCGCGCCTCACCCCCGAACAGGCGGC
GCACGTCGAGGCGCAGCCTGGGGTGCTCGCCGTCCACCCCGACCAGGCGC
GCCAGCTGCACACCACCCATACCCCGGCGTTCCTCCACCTTACCCAGGCT
TCCGGGCTCCTGCCCGCCGCCGCCTCCGGTGGCGCGTCGTCACCCATCGT
CGGGGTGCTCGACACCGGGATCTACCCCATCGGCCGCGGCTCCTTCGCGC
CCACCGACGGGCTCGGCCCGCCGCCCGCGTCCTTCTCCGGCGGATGCGTC
TCCACCGCCTCCTTCAACGCCTCCGCCTACTGCAACAACAAGCTCATCGG
CGCAAAGTTCTTCTACAAGGGATACGAGGCTGCTCTCGGCCACGCCATCG
ATGAGACGGAGGAGTCCAAGTCGCCACTGGACACCGAGGGCCACGGGACC
CACACCGCCTCCACCGCCGCAGGGTCGCCGGTGACCGGCGCCGGGTTCTT
CGACTACGCGCGTGGCCAGGCGGTGGGCATGTCCCCCGCGGCGCACATCG
CCGCGTACAAGATCTGCTGGAAGTCCGGTTGCTACGACTCCGACATCCTC
GCCGCCATGGACGAGGCCGTCGCGGACGGCGTCGACGTCATATCCCTCTC
CGTCGGCGCCGGCGGCTACGCCCCGAGCTTCTTCCGCGACTCCATCGCCA
TCGGCTCCTTCCACGCCGTTAGCAAGGGCATCGTGGTGTCCGCGTCCGCC
GGCAACTCCGGCCCCGGCGAGTACACCGCGACGAACATCGCGCCATGGAT
ACTGACCGTCGGCGCATCTACCATCGACCGCGAATTCCCGGCTGATGTGG
TTCTAGGCAACGGTCAGGTCTACGGCGGCGTGTCCCTGTACTCCGGCGAA
CCCCTGAACTCCACACTGCTCCCGGTGGTGTACGCCGGCGACTGCGGGTC
TCGGCTTTGCATAATCGGCGAGCTCGATCCAGCGAAGGTTTCCGGCAAGA
TCGTTCTGTGTGAGCGTGGGAGCAACGCCCGTGTGGCGAAAGGCGGGGCA
GTGAAGGTGGCCGGCGGTGCCGGCATGATTCTGGTGAACACGGCGGAGAG
CGGCGAGGAGCTGGTTGCCGACTCCCACCTCGTCCCGGCGACAATGGTGG
GGCAGAAATTCGGCGACAAGATCAAGTACTACGTCCAGAGCGATCCGTCG
CCGACGGCGACCATCGTGTTCCGGGGCACGGTCATCGGGAAGTCGCCGTC
CGCGCCGCGCGTCGCGGCGTTCTCGAGCCGGGGCCCCAACTACCGCGCGC
CGGAGATCCTCAAGCCGGACGTCATTGCCCCCGGCGTCAACATCCTCGCG
GCGTGGACCGGCGAGTCTGCGCCCACCGACCTCGACATCGACCCGAGGCG
CGTGGAGTTCAACATCATCTCCGGCACGTCCATGTCGTGCCCGCACGTCA
GCGGCCTCGCCGCGCTGCTCCGCCAGGCGCAACCGGACTGGAGCCCGGCG
GCGATCAAGTCGGCGCTCATGACCACGGCGTACAACGTGGACAACTCCAG
CGCGGTCATCAAGGACCTGGCTACCGGGACCGAGTCGACGCCGTTCGTCC
GTGGCGCCGGCCACGTCGACCCCAACCGCGCGCTCGACCCTGGCCTCGTG
TACGACGCCGGGACCGAAGACTACGTCTCCTTCCTCTGCACGCTCGGCTA
CTCCCCCTCCATCATCTCCCTCTTCACAACAGACGGCTCCGTCGCCAACT
GTTCGACGAAATTCCCCCGCACCGGGGACCTCAACTACCCCGCCTTCGCC
GTCGTCCTATCCTCCTACAAAGATTCAGTCACCTACCACAGGGTGGTGCG
CAACGTCGGCAGCAACGCCAATGCCGTCTACGAAGCCAAGATCGACAGCC
CGTCCGGTGTGGATGTCACGGTGAGCCCAAGCAAGCTGGTGTTCGACGAG
AGCCACCAGAGCCTGTCCTACGACATCACCATCGCCGCGTCGGGTAACCC
GGTGATCGTCGACACCGAGTACACCTTCGGGTCGGTCACCTGGAGCGACG
GCGTGCACGACGTCACTAGCCCCATCGCCGTGACATGGCCGTCGAACGGC
CGAGCAGCATCCATGTAGAGTAGTGTTGGAAATTTGGGTGTCTTCTGGTT
TGGTGGCAATGGGGACAGCTTGTATAGGTCCTTCTTGGACAGAGATCTCC
ACGCATGAGACCAAATCCTTCCATGAAGCTTAGTGCTCCCATGGCTTCAT
GGAAGGGATCGGTTGCCTGTTCATCGCTATGCACATGTGTAACTCACTGG
ATTGGAGTGGTGAATAATTTTATTTATGCTAAATTACCTGGATTCCCATG
CT
SEQ ID No. 6 Rice Os02g0779200 amino acid sequence
MATLRHLAAVLLILFAAASPAAAAAREQSTYILHLAPEHPALRATRVGGG
GGAVFLGRLLRLPRHLRAPRPRLLYSYAHAATGVAARLTPEQAAHVEAQP
GVLAVHPDQARQLHTTHTPAFLHLTQASGLLPAAASGGASSPIVGVLDTG
IYPIGRGSFAPTDGLGPPPASFSGGCVSTASFNASAYCNNKLIGAKFFYK
GYEAALGHAIDETEESKSPLDTEGHGTHTASTAAGSPVTGAGFFDYARGQ
AVGMSPAAHIAAYKICWKSGCYDSDILAAMDEAVADGVDVISLSVGAGGY
APSFFRDSIAIGSFHAVSKGIVVSASAGNSGPGEYTATNIAPWILTVGAS
TIDREFPADVVLGNGQVYGGVSLYSGEPLNSTLLPVVYAGDCGSRLCIIG
ELDPAKVSGKIVLCERGSNARVAKGGAVKVAGGAGMILVNTAESGEELVA
DSHLVPATMVGQKFGDKIKYYVQSDPSPTATIVFRGTVIGKSPSAPRVAA
FSSRGPNYRAPEILKPDVIAPGVNILAAWTGESAPTDLDIDPRRVEFNII
SGTSMSCPHVSGLAALLRQAQPDWSPAAIKSALMTTAYNVDNSSAVIKDL
ATGTESTPFVRGAGHVDPNRALDPGLVYDAGTEDYVSFLCTLGYSPSIIS
LFTTDGSVANCSTKFPRTGDLNYPAFAVVLSSYKDSVTYHRVVRNVGSNA
NAVYEAKIDSPSGVDVTVSPSKLVFDESHQSLSYDITIAASGNPVIVDTE
YTFGSVTWSDGVHDVTSPIAVTWPSNGRAASM
SEQ ID No. 7 Triticum aestivum cultivar Torero
subtilisin protease DNA
CGACGAGACGCTGGAGTCCAAGTCGCCGCTGGACACAGAGGGCCACGGCA
CCCACACCGCTTCCACGGCCGCCGGGTCGCCGGTGGACGGCGCCGGGTTC
TACCAGTACGCGCGCGGGAGGGCCGTCGGCATGGCCCCCACCGCGCGCAT
CGCCGCGTACAAGATCTGCTGGAAGTCCGGCTGCTTCGACTCCGACATAC
TCGCGGCGTTCGACGAGGCCGTCGGCGACGGCGTCAACGTCATCTCGCTC
TCCGTCGGCTCCACCTACGCCGCAGACTTCTACGAGGACTCCATCGCCAT
CGGCGCCTTCGGGGCAGTGAAGAAGGGCATCGTCGTCTCCGCCTCCGCGG
GCAACTCCGGCCCCGGAGAGTACACCGCGAGCAACATCGCGCCGTGGATA
CTGACCGTCGGCGCGTCCACCGTCGACCGTGGGTTCCCCGCCGACGCGGT
GCTCGGCGACGGCAGCGTGTACGGCGGCGTGTCACTGTACGCCGGGGATC
CCTTAAACTCCACGAAGCTGCCCCTCGTGTACGCCGCGGACTGTGGCTCC
CGGCTTTGCCTCATCGGCGAGCTTGACAAGGACAAGGTCGCCGGAAAGAT
GGTCCTTTGTGAGCGCGGAGTCAACGCGCGTGTCGAGAAGGGCGCGGCCG
TCGGGAAGGCCGGCGGAATCGGCATGATTCTCGCCAACACGGAGGAGAGC
GGCGAGGAGCTCATCGCCGACCCCCACCTCATCCCGTCGACAATGGTGGG
GCAGAAGTTCGGCGACAAGATCAGGCACTACGTCAAGACAGACCCGTCCC
CGACGGCGACCATCGTCTTCCACGGCACGGTCATCGGGAAGTCGCCGTCC
GCGCCCCGCGTCGCGTCGTTTTCGAGCCGCGGCCCAAACTCCCGCGCGGC
GGAGATCCTCAAGCCCGACGTCACGGCCCCCGGCGTCAACATACTCGCGG
CCTGGACCGGCGAGGCCTCCCCGACCGACCTCGACATCGACCCGAGGCGC
SEQ ID No. 8 Triticum aestivum cultivar Torero
subtilisin protease amino acid sequence
DETLESKSPLDTEGHGTHTASTAAGSPVDGAGFYQYARGRAVGMAPTARI
AAYKICWKSGCFDSDILAAFDEAVGDGVNVISLSVGSTYAADFYEDSIAI
GAFGAVKKGIVVSASAGNSGPGEYTASNIAPWILTVGASTVDRGFPADAV
LGDGSVYGGVSLYAGDPLNSTKLPLVYAADCGSRLCLIGELDKDKVAGKM
VLCERGVNARVEKGAAVGKAGGIGMILANTEESGEELIADPHLIPSTMVG
QKFGDKIRHYVKTDPSPTATIVFHGTVIGKSPSAPRVASFSSRGPNSRAA
EILKPDVTAPGVNILAAWTGEASPTDLDIDPRRVPFNIISGTSMSCPHVS
GLAALLRQAHPEWSPAAVKSALMTTAYNLDNSGEIIKDLATGTESTPFVR
GAGHVDPNSALDPGLVYDADTADYIGFLCALGYTPSQIAVFTRDGSVADC
LKKPARSGDLNYPAFAAVFSSYKDSVTYHRVVRNVGSDASAVYEAKVESP
AGVDAKVTPAKLVFDEEHRSLAYEITLAVSGNPVIVDAKYSFGSVTWSDG
KHNVTSPIAVTWPESAGAASM
SEQ ID No. 9 Zea mays EST, TC489899, nucleic acid
sequence
CGCATTGACCAATCTGCTCCGGGCACCATGGAGAGGATCAGTGGCCCGCG
CCTCGCTGTCCTGCTCGCTCTCGCCGTCTTCACCGCCGTCGCCGCAGCGG
CCACGGACGAGGTGCGCGCGCAGTCCACCTACATCATCCACCTCGCCCCA
GGCCACCCGGCGCTGTCCGCAGCGCGCGTCAACGGCGGCGACGAGGCGGC
CCTCCGCCGCCTCCTCCCGCGCCGCCTGCGCGCGCCGAGGCCGCGCGTGC
TCTACTCCTACCAGCACGCTGCCACGGGCATCGCCGCGCGGCTCACGCCC
CAGCAGGCGGCGCACGCCGCGGCCGGGGAGGGCGTCCTGGCCGTGTACCC
CGACCAGGCGCGGCAGCTGCACACCACCCACACCCCGGCGTTCCTCCGCC
TAACGGAGGCCGCCGGGCTCCTCCCGGCTGCGACGGGGGGCGCGTCGTCG
TCTGCCGTCGTCGGCGTGCTCGACACCGGGCTCTACCCCATCGGCCGGTC
CTCGTTCGCGGCAGCAGATGGGCTCGGCCCGGCGCCCGCGTCCTTCTCTG
GTGGATGCGTCTCTGCTGGCTCCTTCAACGCGTCCGCCTACTGCAACAGC
AAGCTCATCGGTGCCAAGTCTTCTACCAGGGGTACGAAGCTGCTCTCGGC
CACCCCATCGATGAGACCAAGGAGTCGAAGTCGCCGCTGGACACTGAGGG
CCATGGCACGCACACCGCCTCCACGGCGGCTGGCTCGCCGGTGGCCGGAG
CCGGGTTCTTCGACTACGCCGAGGGGCAGGCCGTGGGCATGGACCCCGGC
GCGCGCATCGCGGCGTACAAGATCTGCTGGACATCCGGATGCTACGACTC
CGATATCCTCGCCGCCATGGACGAGGCCGTCGCTGACGGCGTCGACGTCA
TCTCGCTCTCCGTCGGCGCCAACGGGTACGCCCCCAGCTTCTTCACCGAT
TCCATCGCCATCGGCGCTTTCCACGCGGTAAGCAAGGGCATCGTGGTCTC
CTGCTCCGCCGGCAACTCCGGCCCCGGCGAGTACACCGCCGTCAACATTG
CGCCGTGGATCCTGACCGTCGGCGCGTCCACCATCGACCGCGAGTTCCCC
GCCGATGTAGTTCTCGGCGACGGCCGCGTCTTTGGTGGCGTCTCTCTGTA
TGCCGGTGACCCCCTGGACTCGACTCAGTTGCCTCTGGTGTTCGCCGGGG
ACTGTGGTTCCCCTCTGTGCCTAATGGGCGAGCTCGACTCGAAGAAGGTG
GCCGGCAAGATGGTGCTCTGTCTGCGTGGTAACAACGCTCGTGTCGAGAA
AGGAGCAGCGGTCAAGCTCGCCGGTGGGGTCGGAATGATCCTCGCCAACA
CCGAGGAGAGCGGCGAGGAGCTCATCGCCGACTCCCACCTCGTGCCGGCG
ACTATGGTCGGGCAGAAGTTCGGCGACAAGATCAGGTACTACGTCCAGAC
GGACCCGTCGCCAACGGCGACCATCGTGTTCCGCGGCACAGTCATCGGCA
AGTCGCGGTCCGCGCCTCGAGTGGCGGCGTTCTCGAGCCGAGGCCCCAAC
TACCGCGCACCGGAGATCCTCAAGCCCGACGTCATCGCCCCGGGCGTCAA
CATACTCGCGGCGTGGACCGGCGCCGCCTCCCCCACCGACCTGGACATCG
ACTCGAGGCGCGTGGAATTCAACATCATCTCCGGCACGTCCATGTCCTGC
CCGCACGTGAGCGGCCTCGCCGCGCTGCTCCGCCAGGCGCACCCGGAGTG
GAGCCCCGCGGCGATCAAGTCGGCGCTCATGACCACGGCGTACAACCTGG
ACAACTCCGGGGAAACCATCAAGGACCTCGCGACGGGCGTGGAGTCGACG
CCGTTCGTCCGTGGCGCCGGTCACGTCGACCCCAACGCCGCCCTCGACCC
AGGGCTGGTGTACGACGCCGGCTCCGACGACTATGTCGCCTTCCTCTGCA
CGCTCGGGTACTCTCCGTCGTTGATCTCCATCTTCACGCAGGACGCATCG
GTCGCCGACTGCTCGACGAAATTCGCTCGCCCCGGCGACCTTAACTACCC
TGCCTTCGCCGCCGTCTTCTCCTCCTACCAAGACTCGGTCACCTACCGCC
GGGTGGTGCGCAACGTCGGCAGCAACTCCAGCGCGGTGTACCAGCCGACG
ATCGCCAGCCCGTACGGCGTGGATGTCACGGTGACCCCGAGCAAGCTCGC
GTTCGACGGGAAGCAGCAGAGCCTGGGATACGAAATCACCATCGCAGTGT
CAGGCAACCCGGTGATCGTGGATTCCAGCTACTCGTTCGGATCCATCACC
TGGAGCGACGGCGCGCACGACGTCACGAGCCCCATTGCCGTGACCTGGCC
GTCCAACGGTGGAGCAGCAGCCATGTAGTAGACTGATGCTGTTGCTACTG
TCTACCGCTGTGGGAAGAAGGACAGGGCCATGAGCCCATGAGATCCGAAA
TCTCCACGCCTCCTGCCTGCCATAGGAATAATTTCCTCGACTGAACCACG
CAATAATTCAGCTGCCCCTATCGTGGTTGTGGTGGACCAATGGACCATGC
TTGCAGCTCTCTCTTTTCATTTAGGGGTAGGTTGGTTGGAGCAGCGTATG
TGATTGGCTGCTTGCAAGGCCGTGAGGGTGCCATCCATTATGGCTCATTG
GCGATTGGACGTGTATGAACAAGTTTGTAATGACTAGAAATAATCATTGT
ACCATGTTTTTTTTATGCTGGCGCGTTTATAGCACCATGCTTTGTGTATG
TCACTTGTGTACGCATACTAAAGAGAAAAGCATGGATATGGAACACCTGA
GGGCTTGTTCGGTTATTCCCAAT

What is claimed is:

1. A transgenic plant cell, plant or a part thereof wherein the activity of a SASP polypeptide is inactivated, repressed or down-regulated.

2. A transgenic plant cell, plant or a part thereof according to claim 1 wherein the plant has increased yield.

3. A transgenic plant cell, plant or a part thereof according to claim 1 wherein the expression of a gene encoding a SASP polypeptide is inactivated, repressed or down-regulated.

4. A transgenic plant cell, plant or a part thereof according to claim 3 wherein the gene encoding a SASP polypeptide is from wheat, rice, brassica or zea mays.

5. A transgenic plant cell, plant or a part thereof according to claim 3 wherein the SASP gene encoding a SASP polypeptide comprises a nucleic acid sequence as shown in SEQ ID No. 1, a functional variant, homologue or orthologue thereof.

6. A transgenic plant cell, plant or a part thereof according to claim 5 wherein the functional variant, homologue or orthologue comprises a nucleic acid sequence as shown in SEQ ID Nos. 3, 4, 5, 7 or 9.

7. A transgenic plant cell, plant or a part thereof according to claim 1 wherein the endogenous SASP gene carries a functional mutation.

8. A transgenic plant cell, a plant or a part thereof according to claim 1 wherein expression of the endogenous SASP gene is silenced.

9. A transgenic plant cell, a plant or a part thereof according to a claim 1 derived from a crop plant.

10. A transgenic plant cell, a plant or a part thereof according to claim 1 derived from a monocotyledonous plant.

11. A transgenic plant cell, a plant or a part thereof according to claim 1 derived from a dicotyledonous plant.

12. A transgenic plant tissue, plant, harvested plant material or propagation material of a plant comprising the plant cell according to claim 1.

13. A transgenic plant cell tissue, plant, harvested plant material or propagation material of a plant according to claim 12 wherein said plant is a brassica, wheat, rice or maize.

14. A method for making a transgenic plant with increased yield comprising inactivating, repressing or down-regulating the activity of a senescence associated subtilisin protease (SASP) polypeptide in a plant.

15. A method according to claim 14 wherein the method comprises inactivating, repressing or down-regulating the expression of a gene encoding a SASP polypeptide.

16. A method according to claim 15 wherein the gene encoding a SASP polypeptide is from wheat, rice, brassica or zea mays.

17. A method according to claim 14 wherein the SASP gene comprises a nucleic acid sequence as shown in SEQ ID No. 1, a functional variant, homologue or orthologue thereof.

18. A method according to claim 17 wherein the functional variant, homologue or orthologue comprises a nucleic acid sequence as shown in SEQ ID Nos. 3, 4, 5, 7 or 9.

19. A method according to claim 14 wherein said method comprises introducing a functional mutation in a gene encoding a SASP protein or peptide in a plant.

20. A method according to claim 19 wherein said mutation is introduced using T-DNA insertion or chemical mutagenesis.

21. A method according to claim 20 comprising using TILLING.

22. A method according to claim 14 comprising silencing of the SASP gene.

23. A method according to claim 22 comprising introducing a RNAi, snRNA, dsRNA, siRNA, miRNA, ta-siRNA or cosuppression molecule which targets the SASP gene into a plant.

24. A method for increasing yield comprising making a plant with increased yield as in claim 14.

25. A plant obtained or obtainable by the method of claim 14.

26. An isolated nucleic acid comprising a sequence as shown in SEQ ID No. 1, a functional variant, homologue or orthologue thereof.

27. An isolated nucleic acid comprising according to claim 26 wherein the functional variant, homologue or orthologue comprises SEQ ID No. 9.

28. An expression cassette comprising an isolated nucleic acid according to claim 26.

29. A plant cell, plant or a part thereof with increased yield wherein the activity of a SASP polypeptide is inactivated, repressed or down-regulated and wherein said plant has been generated by methods that do not solely rely on traditional breeding.


SEQUENCE LISTING (26 sequences)

SEQ ID NO: 1 - DNA, 2334 bp, Arabidopsis thaliana
atggctaagctctctctttcctccatcttcttcgtcttccctctcctcctctgtttcttttccccttcttcttcttcatcggatggcttagaatcctacatcgtccatgtgcagagatctcataagccttccctcttctcctcccacaacaactggcacgtctctctccttcgctctctcccttcttctccccaaccagcaacgctgctctactcttattcacgcgccgttcatggcttctccgctcgtctctcccctatccaaaccgccgccctccgccgtcatccttcagtcatctccgttatacctgatcaagcgcgtgagatccacacaactcacacgcctgccttcctcggtttctcccaaaactctggactctggagcaactcaaattacggggaagacgtgatcgtcggcgttttagatactggaatctggccggaacatccaagtttctcggattcaggtctcggtccaattccatctacctggaaaggcgagtgcgagatcggacctgattttcctgcctcatcttgcaatcggaagcttatcggagctcgagcgttttacaggggatatttaacgcaacggaatggaacaaaaaagcatgcagccaaggaatcgagatcgccgcgtgatacagaaggtcatggcacgcacacggcatctacggcagctggatcggtggttgctaacgcgagtttgtaccagtacgcgcgcggaacagctactgggatggcgtcaaaggcgagaatcgccgcttacaaaatctgttggaccggcggatgttacgattccgatatcctcgccgccatggatcaggcggttgccgacggtgttcacgttatctctctctccgtcggagccagcggttccgccccggagtatcacacggactctatagcgatcggagcatttggagccacgcggcacggcatcgtcgtttcttgctccgctgggaattctggtcctaatcctgaaaccgcgacgaacatcgctccatggatcttaaccgttggtgcgtccaccgtcgatagagaattcgccgcaaacgcaatcacgggagacgggaaagtcttcacgggaacatcactgtacgcaggcgaatctctaccggattctcaactttctctggtatattccggcgattgcggaagtagattgtgttaccctgggaaattgaattcatcattggttgaaggcaaaatcgtgctctgtgacagaggaggcaacgcaagagttgagaaaggaagtgcagtcaagctagccggtggtgctggtatgattctggcgaacacagctgaaagcggtgaagaattaaccgccgattcgcatctcgtcccggcgacaatggttggagctaaagctggagatcaaatccgcgactacatcaaaacatcagactctcccactgcaaaaatcagtttcctaggcactttgatcggaccatctcctccttctcccagagtcgccgctttctccagccgtggaccgaatcacttgacaccggttattcttaaaccggacgtgattgctcctggagtcaacattttagccggttggaccgggatggttggtcctaccgatttagatatcgatccaagacgggttcaattcaacatcatctccggtacatcgatgtcgtgcccacacgttagtggactcgccgctctcctccgtaaagctcatcccgattggtcacctgcagcaatcaaatccgcccttgtaaccaccgcttacgatgtcgaaaactccggcgaaccaatcgaggatctcgccaccggtaaatcatcgaactcattcatccacggagctggacacgtcgatccaaacaaagctttgaatcctggtttggtttacgacatcgaggtcaaagagtacgtagctttcctctgcgccgtgggatacgagtttccggggattctagtctttcttcaagatccaactctttacgacgcatgtgaaacgagcaagctaagaaccgccggcgatctcaattacccatctttctccgtggttttcgcatcgaccggggaagttgtgaaatacaaaagggttgtcaaaaacgtgggaagcaatgtcgacgctgtgtacgaagtcggagttaaatctccggcgaatgttgagattgatgtttctccaagcaagcttgcgttcagcaaggagaagagcgtgttggagtatgaagtcacatttaagagcgttgtgctcggcggaggagtcggatccgtgccgggtcatgaattcgggtcgatcgaatggacagacggtgaacacgttgttaagagtccggtggccgtccaatggggtcagggatcagttcagtccttctga

SEQ ID NO: 2 - protein, 774 aa, Arabidopsis thaliana
MAKLSLSSIFFVFPLLLCFFSPSSSSSDGLESYIVHVQRSHKPSLFSSHNNWHVSLLRSLPSSPQPATLLYSYSRAVHGFSARLSPIQTAALRRHPSVISVIPDQAREIHTTHTPAFLGFSQNSGLWSNSNYGEDVIVGVLDTGIWPEHPSFSDSGLGPIPSTWKGECEIGPDFPASSCNRKLIGARAFYRGYLTQRNGTKKHAAKESRSPRDTEGHGTHTASTAAGSVVANASLYQYARGTATGMASKARIAAYKICWTGGCYDSDILAAMDQAVADGVHVISLSVGASGSAPEYHTDSIAIGAFGATRHGIVVSCSAGNSGPNPETATNIAPWILTVGASTVDREFAANAITGDGKVFTGTSLYAGESLPDSQLSLVYSGDCGSRLCYPGKLNSSLVEGKIVLCDRGGNARVEKGSAKLAGGAGMILANTAESGEELTADSHLVPATMVGAKAGDQIRDYIKTSDSPTAKISFLGTLIGPSPPSPRVAFSSRGPNHLTPVILKPDVIAPGVNILAGWTGMVGPTDLDIDPRRVQFNIISGTSMSCPHVSGLAALLRKHPDWSPAAIKSALVTTAYDVENSGEPIEDLATGKSSNSFIHGAGHVDPNKALNPGLVYDIEVKEYVAFLCAVGYEFPGILVFLQDPTLYDACETSKLRTAGDLNYPSFSVVFASTGEVVKYKRVVKNVGSNVDAVYEVGVKSPANVEIDVSPSKLAFSKEKSVLEYEVTFKSVVLGGGVGSVPGHEFGSIEWTDGEHVVKSPVAVQWGQGSVQSF

SEQ ID NO: 3 - DNA, 2331 bp, Brassica rapa
atggctaagctctctctctcctctgtcttcttcgttttccctctcttcctctgtttcttctcgtcgttatcttcttgggatgggttagaatcatacatcgttcatgtgcagagttctcataagccttctctcttctcctcccacgaccattggcacaactctctcctccgctctctaccgtcctctccacaaccggcgacgctcttatactcttactcacgcgccgttcaaggcttctccgctcgtctctcacctacacagaccgccgctcttcgccgtcacacttccgttatctccgttataccagatcaagcgcgtgagattcacaccactcatacaccttccttcctcggtttctcagataactccggtctctggagcaactccaattacggcgaggacgtgatcgtcggcgttctcgacaccggaatctggccggagcatcctagcttctccgattcaggtctcgatccagttccatctacgtggaaaggcgcgtgcgagatcggacctgactttcccgcttcgtcttgcaaccggaagctcatcggagctcgagcgttctataaaggatacttaacgcatcgcaatgggacggtgaaagcagcgaaggaatcgcgatcgccgcgggatacggaaggtcatggcacgcacacggcatccactgcggcaggatcggtggtggcgaacgcgagcttgtaccaatacgcgcgaggagtggcgcgtgggatggcgtcgaaggcgagaatcgcagcttataaaatctgctggacaggtggttgttacgattccgatatcctcgcggccatggatcaggccgttgctgacggtgttcacgtgatctctctttccgttggtgctaacggttacgctcccgagtatcatatggactcaatcgcgattggagcgtttggagccacgcgccacggtatcgttgtttcctgctccgctggaaactctggtcctggtcctcaaaccgcaactaacatcgctccttggatcttaaccgtcggtgcgtccacgatcgatcgagagttctccgcgaacgcaatcaccggcaacgggaaagtcttcaccggaacgtcgctctacgccggcgagcctctccctgattctcagctttctctggtgtattccggcgattgcggaagcagattgtgctacccagggaagctgaacgcgtccttggtggaagggaagatcgttctctgtgacagaggaggtaacgccagagttgagaaaggaagcgccgtcaagatcgccggcggagcagggatgattctcgcgaacacagctgaaagcggggaagagctcaccgccgattcgcatctcgtcccggcgacgatggtcggagctaaagctggagatcaaatccgcgagtacatccaaaagtcagactctcccaccgcaacaatcagcttcttgggcactttgatcggaccttctcctccttctcccagagtcgcggccttctcaagccgtggaccgaatcatataactccggttatccttaaaccggacgtgattgcgccaggagttaatatattagccggttggaccggaatggttggtccaaccgatttggatatcgatccgagacgggttcaattcaatataatctccggtacatcgatgtcgtgcccacacgtgagcggactcgccgctctcctccgtaaagctcatcccgattggtcaccggcggcgatcaaatccgcgctcgttacaaccgcttacgatacagaaaactccggcgaaccaatcgaggatctcgccaccggtaagtcgtcgaactcgttcatccacggagctggacacgtggatccgaacaaagccttgaaccctgggttggtttacgacatcgacgtcaaagactacgtggccttcctctgcgccgtgggatacgagttcccggggattctagtgttccttcaagatccaactctttacaacgcctgcgagacgagcaagctaagaaccgccggcgatctcaattacccgtcgttctccgtcgttttcggatcgagcgtcgatgttgtgaagtacagaagggttgttaagaacgttgggaccaacgttgaggcggtgtacgaagtcggggttaagtctccggcgaacgtggagatcgatgtgtctccgaggaggcttgcgtttagcaagggggagagcgagttggaatacgaagtgacgtttcggagcgttgtgcttggcggaggagttggatccgtaccgggtcatgaattcgggtcgatcgagtggacagacggtgagcacgtcgtcaagagcccggtggctgttcagtggggtcagggatcatcagttcagtcattctga

SEQ ID NO: 4 - DNA, 2328 bp, Brassica rapa
atggccgcgaagctctctctctcctccgtcttaatcgttttctctctcttcctctgtttctcatcgtcatcatcttcctgggatggcttagagtcatacatcgtccatgtgcaaggatctcacaagccttctctcttctcctcccacagccactggcacaactctctcctccgctccctcccatcctctccccaacccgcgactctcctctactcctactcacgcgccgtcaacggcttctccgcgcgtctctcaccttcccagacctccgctctccgtcgccacccttccgtcatctccctaataccagatcaggcgcgtgagatccacaccactcacacccccgccttcctcggcttctccgataactccggtctctggagcaactccaattacggcgaagacgtgatcgtcggcgttctcgataccggaatctggccggagcatcctagcttctccgattcaggtctcgatcccgttccttccacatggaaaggcgcgtgcgagatcggacctgacttcccggcgtcctcctgcaaccggaagctcatcggagctcgagcgttctacaagggatacctaacgcaccgcaacggatcaaagcacgcagaggaatccaaatcgccgagggatacagcaggtcacgggacgcacaccgcgtcaaccgcggctggatccgttgtggtcaacgcgagtttgtaccaatacgcgcgtggcgtggcgcgtggggtggcgtcgaaggcgagaatcgctgcctacaaaatctgttggactggaggttgttacgattcggatatcctcgcggctatggatcaggccgttgcggatggtgtccacgtcatctctctttccgttggcgctaacggcttcgctccggagtatcataaagactctatcgcgatcggagcgtttggagcgatgcgtcacggcatcgtcgtttcttgctccgccggaaactcaggtccgggaccgcaaacggccactaatatcgctccgtggatcctaaccgtcggtgcgtcgacggtggatagagagttcaccgcgaacgcgatcaccggagacgggaaagtcttcaccggaacgtcgctgtacgcaggagagcctctccctgattctcagattcctctggtgtactccggcgattgcggaagcagattgtgctaccccgggaagctgaactcgtcgttggtggaagggaagatcgttctctgtgatagaggaggaaacgcaagagtcgagaaaggaagcgccgtcaagatcggcggcggagcagggatgattctcgcgaacacagctgaaagcggcgaagaactcaccgccgattcgcatctcgtcccggcgacgatggtcggagctaaagccggagatcaaatccgcgactacatcaaaaactcaaactctccaaccgcaacgatcagcttcttgggaactttgatcggcccatctcctccttctccaagagtcgcagccttctctagccgtggaccaaatcacataaccccggttatcctcaaaccggacgtgattgcgccaggtgttaatatattagccggttggaccggaatggttggtccaaccgatttagatatcgacccgagacgagtcaaattcaacatcatctccggtacatcgatgtcgtgcccgcacgtgagcggactcgccgctctcctccgtaaagctcaccccgattggtccccggcggcgatcaaatccgcgctcgtgacaaccgcttacgacaccgaaaactccggggaaccgatcgaggatctcgccaccggtgaatcgtcgaactcgttcatccacggagcgggacacgtggatccgaacaaagcgttgaatcccggtttggtttacgacctcgacgctaaagagtacgtcgcgttcctctgcgccgtggggtacgagttcccggggattctggtgttccttcaagatccgagtctttacgacgcttgtgagacgagcaagcttagaaccgccggggatctcaattacccgtctttctccgtcgttttcggatcgagtgttgatgttgttaagtacaggagagttgttaagaacgtggggagcaatgttgacgcggtgtatcaagtcggagttaaggctccggcgaatgtggagatcgatgtgtctccgagcaagcttgcgtttagtaaagagactagggagatggagtacgaagtgacgtttaagagcgttgtgcttggaggtggagttggatccgttccgggtcatgagttcgggtcgattgagtggacagacggtgaacatgtcgtcaagagtcccgtggctgttcaatggagtcaggggtcagttcagtcattctga

SEQ ID NO: 5 - DNA, 2652 bp, Oryza sativa
cttatttagttctccaggccgcattggcgtcgagtcatcgaccaatccaatccgctcccccggtgcgccatggctaccctccgccatctcgccgccgtgctcctcatcctcttcgccgccgcgtcgccggcggcggcggccgcgagagagcagtcgacgtacatcctccacctcgcgcccgagcacccggcgctcagggccacgcgcgtcggcggcggcggcggcgccgtgttcctcggccgcctccttcgcctcccgcgccatctgcgcgcaccgcggccacggttgctctactcctacgcgcacgcagcgacgggggtcgcggcgcgcctcacccccgaacaggcggcgcacgtcgaggcgcagcctggggtgctcgccgtccaccccgaccaggcgcgccagctgcacaccacccataccccggcgttcctccaccttacccaggcttccgggctcctgcccgccgccgcctccggtggcgcgtcgtcacccatcgtcggggtgctcgacaccgggatctaccccatcggccgcggctccttcgcgcccaccgacgggctcggcccgccgcccgcgtccttctccggcggatgcgtctccaccgcctccttcaacgcctccgcctactgcaacaacaagctcatcggcgcaaagttcttctacaagggatacgaggctgctctcggccacgccatcgatgagacggaggagtccaagtcgccactggacaccgagggccacgggacccacaccgcctccaccgccgcagggtcgccggtgaccggcgccgggttcttcgactacgcgcgtggccaggcggtgggcatgtcccccgcggcgcacatcgccgcgtacaagatctgctggaagtccggttgctacgactccgacatcctcgccgccatggacgaggccgtcgcggacggcgtcgacgtcatatccctctccgtcggcgccggcggctacgccccgagcttcttccgcgactccatcgccatcggctccttccacgccgttagcaagggcatcgtggtgtccgcgtccgccggcaactccggccccggcgagtacaccgcgacgaacatcgcgccatggatactgaccgtcggcgcatctaccatcgaccgcgaattcccggctgatgtggttctaggcaacggtcaggtctacggcggcgtgtccctgtactccggcgaacccctgaactccacactgctcccggtggtgtacgccggcgactgcgggtctcggctttgcataatcggcgagctcgatccagcgaaggtttccggcaagatcgttctgtgtgagcgtgggagcaacgcccgtgtggcgaaaggcggggcagtgaaggtggccggcggtgccggcatgattctggtgaacacggcggagagcggcgaggagctggttgccgactcccacctcgtcccggcgacaatggtggggcagaaattcggcgacaagatcaagtactacgtccagagcgatccgtcgccgacggcgaccatcgtgttccggggcacggtcatcgggaagtcgccgtccgcgccgcgcgtcgcggcgttctcgagccggggccccaactaccgcgcgccggagatcctcaagccggacgtcattgcccccggcgtcaacatcctcgcggcgtggaccggcgagtctgcgcccaccgacctcgacatcgacccgaggcgcgtggagttcaacatcatctccggcacgtccatgtcgtgcccgcacgtcagcggcctcgccgcgctgctccgccaggcgcaaccggactggagcccggcggcgatcaagtcggcgctcatgaccacggcgtacaacgtggacaactccagcgcggtcatcaaggacctggctaccgggaccgagtcgacgccgttcgtccgtggcgccggccacgtcgaccccaaccgcgcgctcgaccctggcctcgtgtacgacgccgggaccgaagactacgtctccttcctctgcacgctcggctactccccctccatcatctccctcttcacaacagacggctccgtcgccaactgttcgacgaaattcccccgcaccggggacctcaactaccccgccttcgccgtcgtcctatcctcctacaaagattcagtcacctaccacagggtggtgcgcaacgtcggcagcaacgccaatgccgtctacgaagccaagatcgacagcccgtccggtgtggatgtcacggtgagcccaagcaagctggtgttcgacgagagccaccagagcctgtcctacgacatcaccatcgccgcgtcgggtaacccggtgatcgtcgacaccgagtacaccttcgggtcggtcacctggagcgacggcgtgcacgacgtcactagccccatcgccgtgacatggccgtcgaacggccgagcagcatccatgtagagtagtgttggaaatttgggtgtcttctggtttggtggcaatggggacagcttgtataggtccttcttggacagagatctccacgcatgagaccaaatccttccatgaagcttagtgctcccatggcttcatggaagggatcggttgcctgttcatcgctatgcacatgtgtaactcactggattggagtggtgaataattttatttatgctaaattacctggattcccatgct

SEQ ID NO: 6 - protein, 782 aa, Oryza sativa
MATLRHLAAVLLILFAAASPAAAAAREQSTYILHLAPEHPALRATRVGGGGGAVFLGRLLRLPRHLRAPRPRLLYSYAHAATGVAARLTPEQAAHVEAQPGVLAVHPDQARQLHTTHTPAFLHLTQASGLLPAAASGGASSPIVGVLDTGIYPIGRGSFAPTDGLGPPPASFSGGCVSTASFNASAYCNNKLIGAKFFYKGYEAALGHAIDETEESKSPLDTEGHGTHTASTAAGSPVTGAGFFDYARGQAVGMSPAAHIAAYKICWKSGCYDSDILAAMDEAVADGVDVISLSVGAGGYAPSFFRDSIAIGSFHAVSKGIVVSASAGNSGPGEYTATNIAPWILTVGASTIDREFPADVVLGNGQVYGGVSLYSGEPLNSTLLPVVYAGDCGSRLCIIGELDPAKVSGKIVLCERGSNARVAKGGAVKVAGGAGMILVNTAESGEELVADSHLVPATMVGQKFGDKIKYYVQSDPSPTATIVFRGTVIGKSPSAPRVAAFSSRGPNYRAPEILKPDVIAPGVNILAAWTGESAPTDLDIDPRRVEFNIISGTSMSCPHVSGLAALLRQAQPDWSPAAIKSALMTTAYNVDNSSAVIKDLATGTESTPFVRGAGHVDPNRALDPGLVYDAGTEDYVSFLCTLGYSPSIISLFTTDGSVANCSTKFPRTGDLNYPAFAVVLSSYKDSVTYHRVVRNVGSNANAVYEAKIDSPSGVDVTVSPSKLVFDESHQSLSYDITIAASGNPVIVDTEYTFGSVTWSDGVHDVTSPIAVTWPSNGRAASM

SEQ ID NO: 7 - DNA, 1000 bp, Triticum aestivum
cgacgagacgctggagtccaagtcgccgctggacacagagggccacggcacccacaccgcttccacggccgccgggtcgccggtggacggcgccgggttctaccagtacgcgcgcgggagggccgtcggcatggcccccaccgcgcgcatcgccgcgtacaagatctgctggaagtccggctgcttcgactccgacatactcgcggcgttcgacgaggccgtcggcgacggcgtcaacgtcatctcgctctccgtcggctccacctacgccgcagacttctacgaggactccatcgccatcggcgccttcggggcagtgaagaagggcatcgtcgtctccgcctccgcgggcaactccggccccggagagtacaccgcgagcaacatcgcgccgtggatactgaccgtcggcgcgtccaccgtcgaccgtgggttccccgccgacgcggtgctcggcgacggcagcgtgtacggcggcgtgtcactgtacgccggggatcccttaaactccacgaagctgcccctcgtgtacgccgcggactgtggctcccggctttgcctcatcggcgagcttgacaaggacaaggtcgccggaaagatggtcctttgtgagcgcggagtcaacgcgcgtgtcgagaagggcgcggccgtcgggaaggccggcggaatcggcatgattctcgccaacacggaggagagcggcgaggagctcatcgccgacccccacctcatcccgtcgacaatggtggggcagaagttcggcgacaagatcaggcactacgtcaagacagacccgtccccgacggcgaccatcgtcttccacggcacggtcatcgggaagtcgccgtccgcgccccgcgtcgcgtcgttttcgagccgcggcccaaactcccgcgcggcggagatcctcaagcccgacgtcacggcccccggcgtcaacatactcgcggcctggaccggcgaggcctccccgaccgacctcgacatcgacccgaggcgc

SEQ ID NO: 8 - protein, 571 aa, Triticum aestivum
DETLESKSPLDTEGHGTHTASTAAGSPVDGAGFYQYARGRAVGMAPTARIAAYKICWKSGCFDSDILAAFDEAVGDGVNVISLSVGSTYAADFYEDSIAIGAFGAVKKGIVVSASAGNSGPGEYTASNIAPWILTVGASTVDRGFPADAVLGDGSVYGGVSLYAGDPLNSTKLPLVYAADCGSRLCLIGELDKDKVAGKMVLCERGVNARVEKGAAVGKAGGIGMILANTEESGEELIADPHLIPSTMVGQKFGDKIRHYVKTDPSPTATIVFHGTVIGKSPSAPRVASFSSRGPNSRAAEILKPDVTAPGVNILAAWTGEASPTDLDIDPRRVPFNIISGTSMSCPHVSGLAALLRQAHPEWSPAAVKSALMTTAYNLDNSGEIIKDLATGTESTPFVRGAGHVDPNSALDPGLVYDADTADYIGFLCALGYTPSQIAVFTRDGSVADCLKKPARSGDLNYPAFAAVFSSYKDSVTYHRVVRNVGSDASAVYEAKVESPAGVDAKVTPAKLVFDEEHRSLAYEITLAVSGNPVIVDAKYSFGSVTWSDGKHNVTSPIAVTWPESAGAASM

SEQ ID NO: 9 - DNA, 2823 bp, Zea mays
cgcattgaccaatctgctccgggcaccatggagaggatcagtggcccgcgcctcgctgtcctgctcgctctcgccgtcttcaccgccgtcgccgcagcggccacggacgaggtgcgcgcgcagtccacctacatcatccacctcgccccaggccacccggcgctgtccgcagcgcgcgtcaacggcggcgacgaggcggccctccgccgcctcctcccgcgccgcctgcgcgcgccgaggccgcgcgtgctctactcctaccagcacgctgccacgggcatcgccgcgcggctcacgccccagcaggcggcgcacgccgcggccggggagggcgtcctggccgtgtaccccgaccaggcgcggcagctgcacaccacccacaccccggcgttcctccgcctaacggaggccgccgggctcctcccggctgcgacggggggcgcgtcgtcgtctgccgtcgtcggcgtgctcgacaccgggctctaccccatcggccggtcctcgttcgcggcagcagatgggctcggcccggcgcccgcgtccttctctggtggatgcgtctctgctggctccttcaacgcgtccgcctactgcaacagcaagctcatcggtgccaagtcttctaccaggggtacgaagctgctctcggccaccccatcgatgagaccaaggagtcgaagtcgccgctggacactgagggccatggcacgcacaccgcctccacggcggctggctcgccggtggccggagccgggttcttcgactacgccgaggggcaggccgtgggcatggaccccggcgcgcgcatcgcggcgtacaagatctgctggacatccggatgctacgactccgatatcctcgccgccatggacgaggccgtcgctgacggcgtcgacgtcatctcgctctccgtcggcgccaacgggtacgcccccagcttcttcaccgattccatcgccatcggcgctttccacgcggtaagcaagggcatcgtggtctcctgctccgccggcaactccggccccggcgagtacaccgccgtcaacattgcgccgtggatcctgaccgtcggcgcgtccaccatcgaccgcgagttccccgccgatgtagttctcggcgacggccgcgtctttggtggcgtctctctgtatgccggtgaccccctggactcgactcagttgcctctggtgttcgccggggactgtggttcccctctgtgcctaatgggcgagctcgactcgaagaaggtggccggcaagatggtgctctgtctgcgtggtaacaacgctcgtgtcgagaaaggagcagcggtcaagctcgccggtggggtcggaatgatcctcgccaacaccgaggagagcggcgaggagctcatcgccgactcccacctcgtgccggcgactatggtcgggcagaagttcggcgacaagatcaggtactacgtccagacggacccgtcgccaacggcgaccatcgtgttccgcggcacagtcatcggcaagtcgcggtccgcgcctcgagtggcggcgttctcgagccgaggccccaactaccgcgcaccggagatcctcaagcccgacgtcatcgccccgggcgtcaacatactcgcggcgtggaccggcgccgcctcccccaccgacctggacatcgactcgaggcgcgtggaattcaacatcatctccggcacgtccatgtcctgcccgcacgtgagcggcctcgccgcgctgctccgccaggcgcacccggagtggagccccgcggcgatcaagtcggcgctcatgaccacggcgtacaacctggacaactccggggaaaccatcaaggacctcgcgacgggcgtggagtcgacgccgttcgtccgtggcgccggtcacgtcgaccccaacgccgccctcgacccagggctggtgtacgacgccggctccgacgactatgtcgccttcctctgcacgctcgggtactctccgtcgttgatctccatcttcacgcaggacgcatcggtcgccgactgctcgacgaaattcgctcgccccggcgaccttaactaccctgccttcgccgccgtcttctcctcctaccaagactcggtcacctaccgccgggtggtgcgcaacgtcggcagcaactccagcgcggtgtaccagccgacgatcgccagcccgtacggcgtggatgtcacggtgaccccgagcaagctcgcgttcgacgggaagcagcagagcctgggatacgaaatcaccatcgcagtgtcaggcaacccggtgatcgtggattccagctactcgttcggatccatcacctggagcgacggcgcgcacgacgtcacgagccccattgccgtgacctggccgtccaacggtggagcagcagccatgtagtagactgatgctgttgctactgtctaccgctgtgggaagaaggacagggccatgagcccatgagatccgaaatctccacgcctcctgcctgccataggaataatttcctcgactgaaccacgcaataattcagctgcccctatcgtggttgtggtggaccaatggaccatgcttgcagctctctcttttcatttaggggtaggttggttggagcagcgtatgtgattggctgcttgcaaggccgtgagggtgccatccattatggctcattggcgattggacgtgtatgaacaagtttgtaatgactagaaataatcattgtaccatgttttttttatgctggcgcgtttatagcaccatgctttgtgtatgtcacttgtgtacgcatactaaagagaaaagcatggatatggaacacctgagggcttgttcggttattcccaat

SEQ ID NO: 10 - protein, 6 aa, Arabidopsis thaliana
SDILAA

SEQ ID NO: 11 - protein, 12 aa, Arabidopsis thaliana
SGTSMSCPHVSG

SEQ ID NO: 12 - protein, 5 aa, Arabidopsis thaliana
GAGHV

SEQ ID NO: 13 - protein, 8 aa, Arabidopsis thaliana
IHTTHTPA

SEQ ID NO: 14 - protein, 5 aa, Arabidopsis thaliana
LSVGA

SEQ ID NO: 15 - protein, 9 aa, Arabidopsis thaliana
ADSHLVPAT

SEQ ID NO: 16 - DNA, 22 bp, Artificial Sequence
  Primer Sequence
gcgtggaccgcttgctgcaact

SEQ ID NO: 17 - DNA, 19 bp, Artificial Sequence
  Primer Sequence
tcggattttctgcattcac

SEQ ID NO: 18 - DNA, 21 bp, Artificial Sequence
  Primer Sequence
ttcttaaaccggacgtgattg

SEQ ID NO: 19 - DNA, 24 bp, Artificial Sequence
  Primer Sequence
ccccggtgcgccatggctaccctc

SEQ ID NO: 20 - DNA, 25 bp, Artificial Sequence
  Primer Sequence
ctacatggatgctgctcggccgttc

SEQ ID NO: 21 - DNA, 15 bp, Artificial Sequence
  Primer Sequence
atggctaagctctct

SEQ ID NO: 22 - DNA, 16 bp, Artificial Sequence
  Primer Sequence
cgctgctttcaccgtc

SEQ ID NO: 23 - DNA, 15 bp, Artificial Sequence
  Primer Sequence
atggccgcgaagctc

SEQ ID NO: 24 - DNA, 17 bp, Artificial Sequence
  Primer Sequence
cctctgcgtgctttgat

SEQ ID NO: 25 - DNA, 22 bp, Artificial Sequence
  Primer Sequence
gaggctattcggctatgactgg

SEQ ID NO: 26 - DNA, 21 bp, Artificial Sequence
  Primer Sequence
atcgggagcggcgataccgta